SEQUENCE LISTING (7 sequences)

SEQ ID NO: 1 - protein, 63 aa, Artificial Sequence
  Description of Artificial Sequence Synthetic polypeptide
MKRRIRRERNKMAAAKSRNRRRELTDTLQAETDQLEDEKSALQTEIANLLKEKEKLEFILAAH

SEQ ID NO: 2 - protein, 63 aa, Artificial Sequence
  Description of Artificial Sequence Synthetic polypeptide
MKAERKRMRNRIAASKSRKRKLERIARLEEKVKTLKAQNSELASTANMLREQVAQLKQKVMNH

SEQ ID NO: 3 - DNA, 18 bp, Artificial Sequence
  Description of Artificial Sequence Synthetic oligonucleotide
ggaaaatttgtttcatag

SEQ ID NO: 4 - DNA, 26 bp, Artificial Sequence
  Description of Artificial Sequence Synthetic oligonucleotide
ctgtatgaaacaaattttcctctttg

SEQ ID NO: 5 - DNA, 17 bp, Artificial Sequence
  Description of Artificial Sequence Synthetic oligonucleotide
tctcctatgactcatcc

SEQ ID NO: 6 - protein, 300 aa, Artificial Sequence
  Description of Artificial Sequence Synthetic polypeptide
MRGSHHHHHHTDPHASSVPLEWPLSSQSGSYELRIEVQPKPHHRAHYETEGSRGAVKAPTGGHPVVQLHGYMENKPLGLQIFIGTADERILKPHAFYQVHRITGKTVTTTSYEKIVGNTKVLEIPLEPKNNMRATIDCAGILKLRNADIELRKGETDIGRKNTRVRLVFRVHIPESSGRIVSLQTASNPIECSQRSAHELPMVERQDTDSCLVYGGQQMILTGQNFTSESKVVFTEKTTDGQQIWEMEATVDKDKSQPNMLFVEIPEYRNKHIRTPVKVNFYVINGKRKRSQPQHFTYHP

SEQ ID NO: 7 - DNA, 30 bp, Artificial Sequence
  Description of Artificial Sequence Synthetic oligonucleotide
ccttctgtatgaaacaaattttcctctttg

(19) United States
(12) Patent Application Publication
(10) Pub. No.: US 2014/0294873 A1
(43) Pub. Date: Oct. 2, 2014

(54) SMALL MOLECULE SCREEN FOR INHIBITORS OF NFAT: AP-1: DNA INTERACTIONS

(71) Applicant: CHILDREN'S MEDICAL CENTER CORPORATION, Boston, MA (US)

(72) Inventors: Srimoyee Ghosh, Brookline, MA (US); Anjana Rao, La Jolla, CA (US); Patrick G. Hogan, La Jolla, CA (US)

(73) Assignee: Children Medical Center Corporation, Boston, MA (US)

(21) Appl. No.: 14/356,795

(22) PCT Filed: Nov. 8, 2012
(86) PCT No.: PCT/US12/64192

§ 371 Date: May 7, 2014

Related U.S. Application Data
(60) Provisional application No. 61/557,092, filed on Nov. 8, 2011.

(51) Int. Cl. C12Q 1/68; A61K 31/15; A61K 45/06; A61K 31/4025; C07D 405/12; C07C 251/84

(52) U.S. Cl. CPC C12Q 1/6816; C07D 405/12; C07C 251/84; A61K 45/06; A61K 31/4025; A61K 31/15; C12Q 2537/137
USPC 424/184.1; 435/6.12; 435/375; 514/422; 514/639

(57) ABSTRACT

The invention provides a screening assay for selecting inhibitors of NFAT:Transcription factor interactions. The invention also provides compositions and methods for inhibiting immune response in a subject.

### CROSS REFERENCE TO RELATED APPLICATIONS

This application claims benefit under 35 U.S.C. §119(e) of U.S. Provisional Patent Application Ser. No. 61/557,092 filed Nov. 8, 2011, content of which is incorporated herein by reference in its entirety.

### GOVERNMENT SUPPORT

This invention was made with government support under grant no. 5T32AI070085-03 and grant no. R01 CA42471 awarded by the National Institutes of Health. The government has certain rights in the invention.

### SEQUENCE LISTING

The instant application contains a Sequence Listing which has been submitted in ASCII format via EFS-Web and is hereby incorporated by reference in its entirety. Said ASCII copy, created on Oct. 31, 2012, 2012, is named 701039PC.txt and is 5,318 bytes in size.

### FIELD OF INVENTION

The invention relates to a screening assay for inhibitors of NFAT:Transcription factor interactions. The invention also relates to compositions and methods for inhibiting immune response in a subject

### BACKGROUND OF THE INVENTION

NFAT is known to drive three disparate transcriptional programs in activated T cells: (i) productive activation through formation of cooperative complexes with its major transcriptional partner, AP-1 (Fos-Jun), culminating in effector T cell responses, (ii) anergy in the absence of AP-1, likely mediated by NFAT dimers and (iii) the suppressor function of regulatory T cells (Tregs) through formation of cooperative complexes with Foxp3, the Treg lineage specification factor. X-ray crystal structures of all three complexes have been solved, revealing the involvement of distinct residues of NFAT at each interface (ref), thus allowing for development of small molecule inhibitors that selectively hamper a specific protein: protein interaction. While NFAT activation is an invariant outcome of T cell receptor (TCR) stimulation, AP-1 activation is contingent upon the triggering of T cell costimulatory receptors (CD28 and integrins), and is therefore associated with full-blown T cell activation.

Conventional immunosuppressants like cyclosporin A and FK506 block the pleiotropic phosphatase calcineurin which is essential for NFAT activation, resulting in a global inhibition of T cell activation, affecting both effector and tolerogenic arms; additionally they block calcineurin activity towards all its intracellular substrates and hence are associated with toxic side-effects including nephrotoxicity and neurotoxicity. However, specific inhibition of NFAT:AP-1 interaction would be expected to selectively block productive immune activation without interfering with tolerogenic programs of T cell anergy and Treg-mediated suppression. Thus, there is a need for modulators of NFAT:Transcription factor interaction that can specifically inhibit NFAT:transcription factor interactions thus selectively attenuate the deleterious immune activation (as in autoimmunity or post-transplantation). Such inhibitors can provide a means for targeted immune intervention.

### SUMMARY OF THE INVENTION

In one aspect the invention provides a screening assay for identifying an agent that modulates NFAT:AP-1:DNA interaction, the assay comprising measuring/detecting/evaluating the formation and/or stability of a complex comprising a NFAT peptide, a Fos peptide, a Jun peptide, and an complexing oligonucleotide in the presence of a test compound relative to a control/reference sample. In some embodiments, the measuring/detecting/evaluating step comprises a FRET based assay.

In another aspect, the invention provides a compound selected by the screening assay described herein.

In yet another aspect the invention provides a method of inhibiting an immune response in a subject, the method comprising administering a therapeutically effective amount of a compound described herein.

### BRIEF DESCRIPTION OF THE FIGURES

Exemplary embodiments are illustrated in referenced figures. It is intended that the embodiments and figures disclosed herein are to be considered illustrative rather than restrictive.

FIGS. 1A-1E show development of a FRET-based high throughput screen for inhibitors of NFAT:AP-1: DNA interaction. FIG. 1A is a schematic depiction of the NFAT:AP-1: DNA complex; OG 488 on a substituted, unique Cys residue proximal to the Fos N-terminal functions as FRET donor; the mARRE2 oligoncucleotide is end-labeled with Alexa 546 towards the non-consensus AP-1 site. The NFAT site is marked in boldface and the AP-1 site is underlined. FIG. 1A discloses SEQ ID NO: 3. FIGS. 1B and 1D shows the dissociation of kinetics of the NFAT:AP-1:DNA complex (in the presence of WT NFAT; green triangles) versus AP-1: DNA alone (absence of NFAT; blue circles or presence of mutant NFAT-RIT; yellow triangles) upon addition of unlabeled Fos (FIG. 1B) and unlabeled consensus AP-1 oligonucleotide (FIG. 1C). FIG. 1D shows the optimized FRET binding assay using 10 nM Fos-OG488/Jun/DNA-Alexa 546/consensus AP-1 oligo and titrating in increasing amounts of NFAT-DBD. FIG. 1E shows the calculation of Z′ factor for statistical robustness of the assay using wild-type NFAT and mutated NFAT (NFAT-RIT).

FIGS. 2A-2D show the performance of the high throughput screen. FIG. 2A shows the results from a sample plate depicting donor fluorescence z-scores on the Y— and well numbers on the X-axes; compound wells scoring as hits are indicated by arrows. FIG. 2B shows the Gaussian distributions of positive and negative controls and sample wells; outliers (wells having z-scores of 2 or more) have been indicated. FIG. 2C shows the correlation between donor fluorescence values for replicate plates. FIG. 2D is a summary of all compounds tested.

FIGS. 3A and 3B show chemical structures, donor dequenching curves and inhibitory effects in a NFAT:AP-1: DNA EMSA for two inhibitory compounds 1668 P02 (FIG. 3A) and 1661 N20 (FIG. 3B) identified as hits in the primary screen. The indicated concentrations of each compound were added to 80 nM NFAT followed by addition of 20 nM Fos-OG/Jun/DNA-Alexa 546/AP-1 consensus oligonucleotide followed by measurement of donor fluorescence. IC50 values of each compound are indicated (top right panels; FIGS. 3A and 3B). Electrophoretic mobility shift assays (EMSAs) were performed by incubating 20 nM NFAT, 10 nM Fos, 10 nM Jun and increasing amounts of either compound; 32P end-labeled ARRE2 was added and the reactions were loaded on a 4% polyacrylamide gel. EMSA band intensities were quantified using the Image J software (bottom left panels; FIGS. 3A and 3B). Both compounds cause modest inhibition of NFAT:AP-1: DNA complex formation in the 0-300 μM range and almost abrogate assembly of the ternary complex at 6.25 mM, leaving the NFAT:DNA complex unaffected; compare duplicate left (DMSO alone; no compound added) and right lanes (6.25 mM of compound); bottom right panels of FIGS. 3A and 3B.

FIGS. 4A and 4B show inhibition of IL-2 and TNFα production in activated CD4 T cells. FIG. 4A shows FACS analysis. Mouse CD4 T cells were stimulated with αCD3/αCD28 for 48 h, allowed to rest for 3 days and restimulated with 10 nM PMA/500 nM ionomycin for 4 h in the presence of indicated concentrations of each compound or DMSO vehicle control. Cells were fixed and stained for intracellular IL-2 (top panel) and TNF [symbol] bottom panel). The solid grey histogram represents the unstimulated control. FIGS. 4B and 4C are line graphs showing MFIs (FIG. 4B) and percentage positive populations (FIG. 4C) for each cytokine measured.

FIGS. 5A-5D show that compounds 1661 N20 and 1668 P02 inhibit production of IL-2 and TNFα but not CD69 upregulation in CD4 T cells. Mouse CD4 T cells were stimulated with αCD3/αCD28 for 48 h, allowed to rest for 3 days and restimulated with 10 nM PMA/100 nM ionomycin for 4 h in the presence of indicated concentrations of each compound or DMSO vehicle control. Cells were fixed and stained for intracellular IL-2 (FIG. 5A top) and TNFα (FIG. 5A bottom). FIG. 5B shows the inhibition curves for % positive and MFIs of IL-2 and TNFα. FIG. 5C shows that there was no apparent change in live cell numbers upon addition of compounds. Data are representative of three independent experiments. FIG. 5D shows upregulation of CD69 was not affected even in the presence of high concentrations of either compound.

FIGS. 6A and 6B show the reduced severity of EAE induced by adoptive transfer of 1661 N20-treated Th1 cells. Mouse 2D2 CD4 T cells were stimulated with αCD3/αCD28 for 60 h under Th1-polarizing conditions in the presence of 7.8 μM of either compound or DMSO control, as labeled. T cells were allowed to rest for 3 d, restimulated with αCD3/αCD28 for 24 h in the presence of compound or DMSO as during primary stimulation and injected intraperitoneally into B6 mice. FIG. 6A shows weight loss (top) and clinical score of neuroinflammation (bottom, for details on the scoring system, see Materials and Methods) following IP injection in mice; *p<0.05 by Student's t-test (two-tailed, unpaired). Mice from the DMSO and 1668 P02-treated groups were euthanized at day 14 following IP injection (indicated by the cross). Uninjected control mice (not shown) did not show clinical symptoms and gained an additional 5-10% of their initial weight through the course of the experiment. FIG. 6B shows quantitation of mRNA levels. RNA was isolated from T cells following a 4 h restimulation in the presence of either compound or DMSO, and mRNA levels were quantified by real time RT-PCR. Gene expression was normalized to an endogenous GAPDH control using the ΔΔCT method. Fold induction was measured against signal from unstimulated DMSO-treated cells.

FIGS. 7A-7C show the purification of OG-labeled Fos (I→C) peptide. FIG. 7A shows purification from lysate of IPTG-induced bacteria by cation exchange chromatography. Fractions were analyzed on a 10-20% Tricine gradient gel. FIG. 7B shows the mass spectrometric analysis of the Fos (I→C) peptide following labeling with OG-maleimide. Unlabeled (˜7.4 kDa) as well as OG-labeled (˜7.9 kDa) peptide species are observed. FIG. 7C shows the mass spectrometric analysis of OG-Fos peptide by further purified by HPLC.

FIG. 8A shows decrease in donor (Oregon Green) fluorescence upon addition of increasing concentration of NFAT to a mixture of 20 nM Fos-OG, mARRE2-Alexa546, and 10 nM Jun; donor fluorescence of Fos-OG alone (F), Fos-OG+DNA (FD), Fos-OG+DNA+Jun (FDJ), and Fos-OG+DNA+Jun+NFAT (FDJN).

FIGS. 8B and 8C are schematic of competition upon addition of unlabeled Fos (FIG. 8b) and unlabeled AP-1 oligonucleotide (FIG. 8C) on the cooperative Fos: Jun: DNA: NFAT complex (I) versus the non-specific Fos: Jun: DNA complex (II).

FIG. 9 shows effect of some selected compounds. The indicated concentrations of 1661 N01 (FIG. 9A), 1666 A08 (FIG. 9B), and 1304 A08 (FIG. 9C) were added to 80 nM NFAT followed by addition of 20 nM Fos-OG/Jun/DNA-Alexa 546/AP-1 consensus oligonucleotide followed by measurement of donor fluorescence.

FIGS. 10A and 10B show the electrophoretic mobility shift assays. EMSAs were performed by titrating in increasing amounts of NFAT to 32P end-labeled ARRE2 (FIG. 10A) and AP-1 to the indicated concentrations of NFAT (2.5 nM, 20 nM) and 32P end-labeled ARRE2 (FIG. 10B).

FIG. 11A illustrate mouse CD4 T cells were stimulated with αCD3/αCD28 for 48 h, allowed to rest for 3 days and restimulated with 10 nM PMA/100 nM ionomycin (left panel) or 10 nM PMA/300 nM ionomycin (right panel) for 4 h in the presence of indicated concentrations of each compound or DMSO vehicle control. Cells were fixed and stained for intracellular IL-2 (white bars) and TNF [symbol] grey bars). Percentage of cells positive for either cytokine (top) and MFIs (bottom) have been graphed; *p<0.05 by Student's t-test comparing with DMSO controls.

FIG. 11B shows the percentage of cells positive for propidium iodide.

FIGS. 12A-12C show mouse CD4 T cells were stimulated with αCD3/αCD28 for 60 h under non-polarizing conditions in the absence of any compound (FIG. 12A); Th1-polarizing conditions in the presence of DMSO or 7.8 μM of each compound (FIG. 12B); and Th17-polarizing conditions in the presence of DMSO or 7.8 μM of each compound (FIG. 12C). T cells were allowed to rest for 3 d and restimulated with αCD3/αCD28 for 4 h in the presence of DMSO or indicated concentrations of each compound. Cells were fixed and stained for intracellular IL-2 and TNFα Primary (10) and secondary stimulation (20) conditions in FIGS. 12B and 12C have been indicated.

FIG. 13 shows quantitation of mRNAs. Mouse CD4 T cells were stimulated with αCD3/αCD28 for 48 h in the presence of TGFβ (iTreg—polarizing conditions) and either DMSO, 7.8 μM 1668 P02, 7.8 μM 1661 N20 or 2 μM CsA. Total RNA was isolated from the cells followed by quantification of mRNA by real time RT-PCR.

FIGS. 14A and 14B show the docking of 1668 P02 (FIG. 14A) and 1661 N20 (FIG. 14B) to the NFAT:AP-1 interface.

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001), and March, Advanced Organic Chemistry Reactions, Mechanisms and Structure 5^{th} ed., J. Wiley & Sons (New York, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

The inventors have discovered inter alia that compounds that inhibit formation and/or stability of a NFAT:AP-1:DNA complex can also block NFAT:AP-1 interactions. Accordingly, in one aspect the invention provides screening assay for identifying an agent that modulates NFAT:AP-1:DNA interaction, the assay comprising measuring/detecting/evaluating the formation and/or stability of a complex comprising a NFAT peptide or a conservative variant thereof, a Fos peptide or a conservative variant thereof, a Jun peptide or a conservative variant thereof, and a complexing oligonucleotide in the presence of a test compound relative to a reference or control sample.

Without wishing to be bound by a theory, the amount or stability of complex in the presence of the test compound decreases relative to a control or reference sample, and the decrease is indicative of a decrease in the formation of the complex. Accordingly, in some embodiments, the test compound inhibits the formation or stability of the complex by at least 5%, at least 10%, at least 20%, at least 30%, at least 40%, at least 50%, at least 60%, at least 70%, at least 80%, at least 90%, at least 95%, at least 98%, or 100% (e.g. complete inhibition) relative to a control sample. A control or reference sample can be one or more of a sample not exposed to the test compound; a sample exposed to known inhibitor of NFAT:AP-1 interaction; a sample exposed to known inhibitor of the complex; or a sample exposed to an excess amount of an unlabeled binding member of the complex. Methods of determining formation and/or stability of the complex are described below.

As used herein, a “NFAT peptide” refers to a peptide which comprises a DNA-binding domain (DBD).

As used herein, the term “DNA-binding domain” refers to a domain corresponding to a region in an amino acid sequence of a family member of Nuclear factor of activated T-cells (NFAT) transcription factors, which binds to a predetermined sequence of an oligonucleotide. Without limitations, a DNA-binding domain derived from any of various species including human, mouse, rat, guinea pig and the like can be used. The “DNA-binding domain” according to the present invention encompasses a variant formed of an amino acid sequence obtained as a result of deletion, addition or substitution of one or more amino acids (for example, 1 to several amino acids (e.g., 6 amino acids)) with respect to the amino acid sequence of the above-mentioned DNA-binding domain to the degree at which the inherent functions of the DNA-binding domain are not lost. In one embodiment, the NFAT peptide comprises the amino acid sequence MRGSHHHHHHTDPHASSVPLEWPLSSQSGSYELRIEVQPKPHHRAHYETEGSRGAVKAPTGG HPVVQLHGYMENKPLGLQIFIGTADERILKPHAFYQVHRITGKTVTTTSYEKIVGNTKVLEIPL EPKNNMRATIDCAGILKLRNADIELRKGETDIGRKNTRVRLVFRVHIPESSGRIVSLQTASNPIE CSQRSAHELPMVERQDTDSCLVYGGQQMILTGQNFTSESKVVFTEKTTDGQQIWEMEATVD KDKSQPNMLFVEIPEYRNKHIRTPVKVNFYVINGKRKRSQPQHFTYHP (SEQ ID NO: 6), or a conservative variant thereof.

The Fos peptide and Jun peptide comprise a Basic Leucine Zipper Domain (bZIP domain). bZIP domain is found in many DNA binding eukaryotic proteins. One part of the domain contains a region that mediates sequence specific DNA binding properties and the Leucine zipper that is required for the dimerization of two DNA binding regions. The DNA binding region comprises a number of basic amino acids such as arginine and lysine. bZIP domain containing proteins include AP-1 fos/jun heterodimer that forms a transcription factor; Jun-B transcription factor; CREB cAMP response element transcription factor; and OPAQUE2 (O2) transcription factor of the 22-kD zein gene that encodes a class of storage proteins in the endosperm of maize {Zea Mays) kernels. The leucine zipper contains an alpha helix with a leucine at every 7th amino acid. If two such helices find one another, the leucine residues can interact as the teeth in a zipper, allowing dimerization of two peptides When binding to the DNA, basic amino acid residues bind to the sugar-phosphate backbone while the helices sit in the major grooves. As used herein, the term “bZIP domain” encompasses a variant formed of an amino acid sequence obtained as a result of deletion, addition or substitution of one or more amino acids (for example, 1 to several amino acids (e.g., 6 amino acids)) with respect to the amino acid sequence of an identified bZIP domain to the degree at which the inherent functions of the DNA-binding domain and dimerization domain are not lost. Further, without limitations, a bZIP domain derived from any of various species including human, mouse, rat, guinea pig and the like can be used.

As used herein, a “Fos peptide” refers to a peptide which comprises a bZIP domain from c-Fos. In one embodiment, the Fos peptide comprises the amino acid sequence MKRRIRRERNKMAAAKSRNRRRELTDTLQAETDQLEDEKSALQTEIANLLKEKEKLEFILAA H (SEQ ID NO: 1) or a conservative variant thereof.

As used herein, a “Jun peptide” refers to a peptide which comprises a bZIP domain from c-Jun. In one embodiment, the Jun peptide comprises the amino acid sequence MKAERKRMRNRIAASKSRKRKLERIARLEEKVKTLKAQNSELASTANMLREQVAQLKQKV MNH (SEQ ID NO: 2) or a conservative variant thereof.

Without limitations, each of the NFAT, Fos, and Jun peptides can independently comprise one or more of the peptide modifications described herein.

As used herein, a “complexing oligonucleotide” refers to an oligonucleotide which comprises a NFAT binding site and a transcription factor binding site. A complexing oligonucleotide can be single-stranded, double-stranded, or a hairpin. Preferably, a complexing oligonucleotide is double stranded. A compelxing oligonucleotide can be DNA, RNA or a chimeric (comprising both deoxy and ribose nucleotides) or comprise one or more oligonucleotide modifications described herein.

As used herein, the term “NFAT binding” site refers to a nucleic acid sequence which is recognized and bound by a DNA binding domain from a NFAT family member. The NFAT binding site can be drawn from a specific gene (i.e., a naturally-occurring NFAT binding element) or it can be a consensus NFAT element, a set of repeated consensus elements, or a set of repeated naturally-occurring NFAT binding elements. Additionally, the NFAT binding site can be from any of various species including human, mouse, rat, guinea pig and the like. In some embodiments, the NFAT binding site comprises the nucleotide sequence 5′-GGAAA-3′.

As used herein, the term “transcription factor binding site” refers to a nucleic acid sequence that is recognized and bound by a transcription factor and mediates the transactivation of a reporter gene in response to that binding. Without limitations, a transcription binding site can be from any of various species including human, mouse, rat, guinea pig and the like. In some embodiments, the transcription factor binding site is an AP-1 binding site. The consensus AP-1 binding site is well known in the art. The AP-1 binding site can be drawn from a specific gene (i.e., a naturally-occurring AP-1 binding element) or it can be a consensus AP-1 element, a set of repeated consensus elements, or a set of repeated naturally-occurring AP-1 binding elements. In some embodiments, the transcription binding site is a non-consensus AP-1 binding site.

In some embodiments, the transcription binding site comprises the nucleotide sequence 5′-TGTTTCA-3′ or 5′-TGAG-3′ or 5′-GATTTG-3′.

The NFAT binding site and the transcription binding site can be arranged in any order in the complexing oligonucleotide, i.e., NFAT binding site in the 5′-terminal and transcription factor binding site in the 3′-terminal or vice versa. Additionally, the two binding sites can be separated by a nucleotide sequence of 1, 2, 3, 4, 5, 6, 7, 8, 9, 10 or more nucleotides or a non-nucleotide linker.

In some embodiments, the complexing oligonucleotide comprises the nucleotide sequence 5′-GGA AAA TTT GTT TCA TAG-3′ (SEQ ID NO: 3) or 5′-CTG TAT GAA ACA AAT TTT CCT CTT TG-3′ (SEQ ID NO: 4).

A variety of assay formats can be used and, in light of the present disclosure, those not expressly described herein will nevertheless be understood by one of ordinary skill in the art. Assay formats which approximate such conditions as formation of peptide: oligonucleotide complexes can be generated in many different forms, and include assays based on cell-free systems, e.g., purified peptides and oligonucleotides or cell lysates, as well as cell based assays which utilize intact cells and in vivo assays.

The components of the complex can be added simultaneously to the assay sample, e.g., a reaction mixture or they can be added sequentially in any order, e.g., forming a mixture of the first component with a second component, adding the third component forming a mixture of the first, second, and third component, and adding the fourth component. In some embodiments, some components of the complex are sequentially while others are added simultaneously, e.g., forming a reaction mixture with a first component with a second component and adding the third and fourth component simultaneously to the mixture, or adding a first component followed by simultaneous addition, e.g., a reaction mixture, of the rest of the components.

The test compound can already be present in the assay sample before addition of the complex components or added afterwards. When the complex components are added in a sequential order, a test compound can be added at any point before all the components have been added to the sample. In some embodiments, the test compound is added before all the complex components have been added to the assay sample. In some embodiments, the test compound is added after at least one of the complex components is added to the assay sample.

Assaying in the presence and absence of a test compound, can be accomplished in any vessel suitable for containing the reactants. Examples include microtitre plates, test tubes, and micro-centrifuge tubes. Alternatively, the sample can include cells in culture, e.g., purified cultured or recombinant cells, or in vivo in an animal subject.

In some embodiments, the screening assays described herein, can be performed in vitro using isolated/purified complex components. In such a system, each component of the screen can be added separately in wells of a multi-well plate, for example 96, 384, and 1536 well plates. In some embodiments, the multimeric complex will be allowed to form prior to the addition of the test compound to be screened. In other embodiments, the members of the complex and the test agent will be added together, e.g., at the same time or simultaneously, with one or more of the members of the complex.

In one embodiment, the NFAT peptide is added first followed by the compound to be tested. The other components of the complex (i.e., the complexing oligonucleotide, the Fos peptide and the Jun peptide) are added afterwards, e.g., as a reaction mixture.

Complex components can be used at any concentration suitable for the assay conditions being used, e.g., size of the reaction vessel, time limitations, detection limits, amount of the limiting component, etc. . . . . Additionally, amount of the various components of the complex can be the same, all different, or combinations thereof. Generally, amount of each component is within 15%, within 10, or within 5% of the amount of each of the other components.

In some embodiments, concentration of each of the complex components in the assay sample is independently from about 0.01 nM to about 1000 nM. In some embodiments, concentration of each of the complex component is independently from about 0.1 nM to about 100 nM, from about 0.5 nM to about 50 nM, or from about 1 nM to about 25 nM. In some embodiments, concentration of each of the complex component is independently from about 10 nM to about 40 nM. In one embodiment, concentration of each of the complex component is about 10 nM. In one embodiment, concentration of each of the complex component is about 20 nM.

The assay sample volume depends on the particular setup being used for the screening assay. Generally, the assay sample has a final volume of about 1 nl to about 1 ml. In some embodiments, the final volume of the sample assay is from about 1 μl to about 100 μl.

In addition to the complex component, a variety of other reagents can be included in the screening assay samples. These include reagents like salts, neutral proteins, e.g., albumin, detergents, etc that are used to facilitate optimal protein-protein binding and/or reduce nonspecific or background interactions. Reagents that improve the efficiency of the assay, such as protease inhibitors, single-stranded nuclease inhibitors, anti-microbial compounds can also be used. The mixture of components is added in any order that provides for the requisite assembly of the complex.

In some embodiments, the assay sample comprises a competitor of at least one component of the complex. The term “competitor of a component of the complex” refers to a compound or composition that competes with said component for binding with another component of the complex. For example, an unlabeled complex component can be considered a competitor of the labeled component. In another example, an oligonucleotide comprising either a NFAT binding site or a transcription binding site is a competitor of the oligonucleotide comprising both a NFAT binding site and a transcription binding site. In some embodiments, the competitor is a Fos-peptide, a Jun-peptide, a NFAT peptide, an oligonucleotide comprising a NFAT binding site, or an oligonucleotide comprising a transcription binding site.

In some embodiments, the competitor is an oligonucleotide comprising a consensus AP-1 binding site. In one embodiment, the competitor is an oligonucleotide comprising the nucleotide sequence 5′-TCT CCT ATG ACT CAT CC-3′(SEQ ID NO: 5). The competitor oligonucleotide can be single-stranded, double-stranded or hairpin. Furthermore, the competitor oligonucleotide can be DNA, RNA or a chimeric (comprising both deoxy and ribose nucleotides).

In some embodiments, the competitor is a Fos protein.

Amount of a competitor of a component of the complex in the sample assay can be adjusted to optimize the detection of the assay. Accordingly, in some embodiments, concentration of the competitor in the assay sample is from about 0.01 nM to about 1000 nM. In some embodiments, amount of the competitor in the assay sample is from about 0.1 nM to about 100 nM, from about 0.5 nM to about 50 nM, or from about 1 nM to about 25 nM. In some embodiments, amount of the competitor in the assay sample is from about 10 nM to about 20 nM. In some embodiments, amount of the competitor in the assay sample is from about 150 nM to about 250 nM. In one embodiment, concentration of the competitor in the assay sample is about 10 nM, about 20 nM, or about 200 nM.

In some embodiments, the competitor is an oligonucleotide and present at a concentration of about 10 nM to about 20 nM in the assay. In one embodiment, the competitor is an oligonucleotide and present at a concentration of about 10 nM in the assay.

In some embodiments, the competitor is a peptide and present at a concentration of about 150 nM to about 250 nM in the assay. In one embodiment, the competitor is a peptide and present at a concentration of about 200 nM in the assay.

Amount of a competitor of a component of the complex in the sample assay can be relative to a component of the complex. For example, the competitor can be present in an amount which is at least 0.1×, at least 0.2×, at least 0.3×, at least 0.4×, at least 0.5×, at least 0.6×, at least 0.7×, at least 0.8x, at least 0.9×, at least lx, at least 1.25×, at least 1.5×, at least 1.75×, at least 2×, at least 2.5×, at least 3×, at least 4×, at least 5× or more relative to the amount of a complex component. In some embodiments, the competitor can be present in an amount which is at least 0.1×, at least 0.2×, at least 0.3×, at least 0.4×, at least 0.5×, at least 0.6×, at least 0.7×, at least 0.8×, at least 0.9×, at least lx, at least 1.25×, at least 1.5×, at least 1.75×, at least 2×, at least 2.5×, at least 3×, at least 4×, at least 5× or more relative to the amount of the complex component being competed against.

Without wishing to be bound by theory any suitable buffer/media/solvent can be used in the screening assay. Exemplary buffers include, but are not limited to, phosphate buffered saline (PBS), sodium phosphate, sodium sulphate, Tris buffers, Tris-HCl buffers, glycine buffer, and sterile water. The buffer can be present in the assay sample at any suitable concentration. Typically, the assay sample comprises a buffer in a concentration of about 5-100 mM. In some embodiments, the assay sample comprises a buffer in a concentration of about 5-75 mM (e.g., 10 mM, 20 mM, 25 mM, 30 mM, 35 mM, 40 mM, 45 mM, or 50 mM). Generally, the buffer has a pH of from about 5 to about 10. In some embodiments, the buffer has a pH of from about 6 to about, from about 6.5 to about 8, or from 6.5 to about 7.5. The pH of the buffer can be adjusted by addition of any suitable acid or base (e.g., HCl). In some embodiments, the sample buffer is 20 mM Tris pH 7.5, 50 mM NaCl, 5% glycerol, 0.5 mg/ml BSA.

Formation and/or stability of the complex can be assayed at any suitable temperature. Accordingly, in some embodiments, the formation and/or stability of the complex is assayed at a temperature in the range of about 15° C. to about 65° C. In some embodiments, the formation and/or stability of the complex is assayed at a temperature in the range of about 15° C. to about 45° C. In some embodiments, the formation and/or stability of the complex is assayed at a temperature in the range of about 15° C. to about 25° C.

After all of the reagents have been added, evaluation of the complex formation or stability can be done right away, e.g., within 5 minutes of addition of last reagent, or after a period of time has elapsed after addition of the last reagent. In some embodiments, the sample assay is allowed to incubate for at least 5 minutes, at least 10 minutes, at least 15 minutes, at least 20 minutes, at least 25 minutes, at least 30 minutes, at least 35 minutes, at least 40 minutes, at least 45 minutes, at least 1 hour, at least 2 hours, at least 5 hours or more before evaluating the complex formation or stability. In one embodiment, the assay sample is allowed to incubate for 30 minutes after addition of the last reagent before evaluating the complex formation or stability.

Without wishing to be bound by a theory, the assays of the invention can evaluate and/or detect a change in complex formation and/or complex stability by detecting one or more of: a change in the binding or physical formation of the complex itself, e.g., by biochemical detection, affinity based detection (e.g., Western blot, affinity columns), immunoprecipitation, fluorescence resonance energy transfer (FRET)-based assays (e.g., FRET or Time Resolved FRET (TR-FRET) assays), surface plasmon resonance (SPR), spectrophotometric means (e.g., circular dichroism, absorbance, and other measurements of solution properties); a change, e.g., an increase or a decrease, in signal transduction, e.g., phosphorylation and/or transcriptional activity; a change, e.g., increase or decrease, cell function.

In general, where the assay is an assay involving fluorescent emission, one or more of the complex components can be joined to a label. The label can be attached directly or indirectly to provide a detectable signal when brought to close proximity. Various labels include radioisotopes, fluorescers, chemiluminescers, enzymes, specific binding molecules, particles, e.g., magnetic particles, and the like. Specific binding molecules include pairs, such as biotin and streptavidin, digoxin and antidigoxin. For the specific binding members, the complementary member would normally be labeled with a molecule that provides for detection, in accordance with known procedures.

Methods of linking ligands, e.g., labels such as a fluorophore, to peptides and oligonucleotides are well known in the art and available to an artisan. See, for example, G. T. Hermanson, Bioconjugate Techniques, 2nd Edition, Academic Press, (2008); J. Kalia & R. T. Raines, Current Organic Chemistry, 14: 138-147 (2010); Bioconjugation Protocols, Methods in Molecular Biology, Vol. 751, 2nd Edition, S. S. Mark (Ed.), Humana Press (2011); Bioconjugation Protocols: Strategies and Methods (Methods in Molecular Biology), C. M. Niemeyer (Ed.), Humana Press (2004); and Molecular Probes Handbook, A Guide to Fluorescent Probes and Labeling Technologies, 11th Edition, I. Johnson & M. T. Z. Spence (Eds.), Invitrogen, content of all of which is incorporated herein by reference. Common types of chemistry for linking a ligand with a peptide include, but are not limited to, amine coupling of lysine amino acid residues (typically through amine-reactive succinimidyl esters), sulfhydryl coupling of cysteine residues (via a sulfhydryl-reactive maleimide), and photochemically initiated free radical reactions.

A molecule, e.g. a fluorophore, can be conjugated with a peptide or oligonucleotide covalently or non-covalently. The covalent linkage between the molecule and the peptide or the oligonucleotide can be mediated by a linker. The non-covalent linkage between the molecule and the peptide or the oligonucleotide can be based on ionic interactions, van der Waals interactions, dipole-dipole interactions, hydrogen bonds, electrostatic interactions, and/or shape recognition interactions.

Without limitations, a fluorophore can be coupled to a peptide or an oligonucleotide at various places, for example, at the N-terminus, C-terminus, and/or at an internal position (e.g., side chain of an amino acid) of the peptide, or at the 5′-terminus, 3′-terminus, and/or at an internal position of the oligonucleotide. Generally, the fluorophore is located within 1-10 residues from an end of the peptide or the oligonucleotide.

In some embodiments, the ligand is attached to the peptide or the oligonucleotide via a linker.

In some embodiments, the ligand can be present on a monomer when said monomer is incorporated into a peptide or oligonucleotide during synthesis. In some embodiments, the ligand can be incorporated via coupling to a “precursor” monomer after said “precursor” monomer has been incorporated into the peptide or the oligonucleotide.

In some embodiments, a change in the formation or stability of the complex is detected by a Fluorescence Resonance Energy Transfer (FRET) based assay. FRET-based assays are described, for example, in U.S. Pat. No. 5,981,200, content of which is incorporated herein by reference in its entirety. FRET requires at least two dye molecules: a first dye that serves as a FRET donor and a second dye that serves as a FRET acceptor. Typically, a FRET donor is an energy donor and a FRET acceptor is an energy acceptor. FRET is the energy transfer that takes place between the FRET donor and the FRET acceptor, as described in more detail below, and is the signal that is measured during a so-called FRET assay.

Fluorescent molecules having the proper emission and excitation spectra that are brought into close proximity with one another can exhibit FRET. FRET is the transfer of energy from a FRET donor to a FRET acceptor. This process occurs as follows: First, a FRET donor is excited, for example, using a picosecond laser pulse, and is converted, by absorption of energy in the form of a photon, from a ground state into an excited state. Second, the FRET donor emits this newly absorbed energy as fluorescent light. Third, if the excited donor molecule is close enough to a suitable acceptor molecule, the excited state can be transferred from the donor to the acceptor in the form of fluorescent light. This energy transfer is known as FRET. Fourth, FRET results in a decrease in the fluorescence or luminescence of the donor and, if the acceptor is itself luminescent, results in an increased luminescence of the acceptor. The light emitted by the acceptor can be measured using a FRET-detection system, and is proportional to the FRET.

Accordingly, FRET can be manifested as a reduction in the intensity of the fluorescent signal from the donor, reduction in the lifetime of its excited state, and/or re-emission of fluorescent light at the longer wavelengths (lower energies) characteristic of the acceptor. Thus, FRET can be manifested as (i) a reduction in the intensity of the fluorescent signal from the FRET donor; (ii) a reduction in the lifetime of the excited state of the FRET donor; and/or (iii) re-emission of fluorescent light typically at the longer wavelengths (lower energies) characteristic of the acceptor. The information gathered can be used for qualitative and quantitative analysis. In some embodiments, the light emitted from the donor will be of a different wavelength than the light emitted from the acceptor.

The efficiency of FRET, i.e., the signal produced when energy is transferred from the donor to the acceptor dye is dependent on the distance (1/d) between the donor and acceptor dye and FRET only occurs efficiently when the donor and acceptor are very close together. Generally, when the fluorophores are physically separate, FRET effects are diminished or eliminated. The decrease in signal depends on the sixth power of the separation distance. Thus, FRET measures distance dependent interactions. Measurements made using FRET are on the scale of about 15 Å to about 100 Å. As used herein, distance dependent interaction means changes in the distance between components of the complex that can be detected by FRET measurement.

In some embodiments, a detected decrease in the FRET signal, e.g., a decrease of at least 0.5%, at least 1%, at least 1.5%, at least 2%, at least 2.5%, at least 3%, at least 5%, at least 10%, at least 15%, at least 20%, at least 25%, at least 30%, at least 35%, at least 40%, at least 50%, or higher relative to a reference or control sample is indicative that a test compound is an inhibitor of the NFAT:transcription factor interaction.

In order to detect this interaction, it is necessary that a FRET donor as well as a FRET acceptor are coupled to one or more of the complex components and that the interaction between these one or more components changes in the presence of a test compound leading to a change in the distance between the FRET donor and the FRET acceptor relative to in the absence of the test compound, a control or a reference sample.

Energy acceptors can either be selected such that they suppress the energy released by the donor, which are referred to as quenchers, or the fluorescence resonance energy acceptors can themselves release fluorescent energy, i.e., they fluoresce. Such energy acceptors are referred to as fluorophore groups or as fluorophores. Metallic complexes are suitable as fluorescence energy donors as well as fluorescence energy acceptors. Fluorophores chosen for use in FRET are generally bright and occur on a timescale ranging from 10˜9 seconds to 10˜4 seconds. Such brightness and timescale facilitate the detection of FRET and allow the use of a variety of detection methods. Exemplary fluorophores include, but are not limited to, those shown in Table 1.

TABLE 1
Exemplary fluorophores

| | Excitation | Emission |
|---|---|---|
| Fluorophore | (nm) | (nm) |
| 1,5 IAEDANS | 336 | 490 |
| 1,8-ANS | 372 | 480 |
| 4-Methylumbelliferone | 385 | 502 |
| 5-carboxy-2,7-dichlorofluorescein | 504 | 529 |
| 5-Carboxyfluorescein (5-FAM) | 492 | 518 |
| 5-Carboxynapthofluorescein (pH 10) | 512/598 | 563/668 |
| 5-Carboxytetramethylrhodamine | 542 | 568 |
| (5-TAMRA) | | |
| 5-FAM (5-Carboxyfluorescein) | 492 | 518 |
| 5-Hydroxy Tryptamine (HAT) | 370-415 | 520-540 |
| 5-ROX (carboxy-X-rhodamine) | 567, 578 | 591, 604 |
| 5-TAMRA | 542, 548 | 552, 568 |
| (5-Carboxytetramethylrhodamine) | | |
| 6-Carboxyrhodamine 6G | 518 | 543 |
| 6-CR 6G | 518 | 543 |
| 6-JOE | 520 | 548 |
| 7-Amino-4-methylcoumarin | 351 | 430 |
| 7-Aminoactinomycin D (7-AAD) | 546 | 647 |
| 7-Hydroxy-4-methylcoumarin | 360 | 449, 455 |
| 9-Amino-6-chloro-2-methoxyacridine | 412, 430 | 471, 474 |
| ABQ | 344 | 445 |
| Acid Fuchsin | 540 | 630 |
| ACMA (9-Amino-6-chloro- | 412, 430 | 471, 474 |
| 2-methoxyacridine) | | |
| Acridine Orange | 460, 520 | 650, 526 |
| Acridine Red | 455-600 | 560-680 |
| Acridine Yellow | 470 | 550 |
| Acriflavin | 436 | 520 |
| Acriflavin Feulgen SITSA | 355-425 | 460 |
| Aequorin (Photoprotein) | | 466 |
| Alexa Fluor 350 ™ | 342, 346 | 441, 442 |
| Alexa Fluor 430 ™ | 431 | 540 |
| Alexa Fluor 488 ™ | 495, 492 | 519, 520 |
| Alexa Fluor 532 ™ | 531, 532 | 553, 554 |
| Alexa Fluor 546 ™ | 556, 557 | 572, 573 |
| Alexa Fluor 568 ™ | 577, 578 | 603 |
| Alexa Fluor 594 ™ | 590, 594 | 617, 618 |
| Alexa Fluor 633 ™ | 632 | 650 |
| Alexa Fluor 647 ™ | 647 | 666 |
| Alexa Fluor 660 ™ | 668 | 698 |
| Alexa Fluor 680 ™ | 679 | 702 |
| Alizarin Complexon | 530-560, 580 | 580, 624-645 |
| Alizarin Red | 530-560 | 580 |
| Allophycocyanin (APC) | 630, 645 | 655, 660 |
| AMC, AMCA-S | 345 | 445 |
| AMCA (Aminomethylcoumarin) | 345, 347 | 425, 444 |
| AMCA-X | 353 | 442 |
| Aminoactinomycin D | 555 | 655 |
| Aminocoumarin | 346, 350 | 442, 445 |
| Anilin Blue | | 600 |
| Anthrocyl stearate | 360-381 | 446 |
| APC-Cy7 | 625-650 | 755 |
| APTS | 424 | 505 |
| Astrazon Brilliant Red 4G | 500 | 585 |
| Astrazon Orange R | 470 | 540 |
| Astrazon Red 6B | 520 | 595 |
| Astrazon Yellow 7 GLL | 540 | 480 |
| Atabrine | 436 | 490 |
| ATTO-TAG ™ CBQCA | 465 | 560 |
| ATTO-TAG ™ FQ | 486 | 591 |
| Auramine | 460 | 550 |
| Aurophosphine G | 450 | 580 |
| Aurophosphine | 450-490 | 515 |
| BAO 9 (Bisaminophenyloxadiazole) | 365 | 395 |
| BCECF (high pH) | 492, 503 | 520, 528 |
| BCECF (low pH) | 482 | 520 |
| Berberine Sulphate | 430 | 550 |
| Beta Lactamase | 409 | 447, 520 |
| BFP blue shifted GFP (Y66H) | 381, 382, 383 | 445, 447, 448 |
| BG-647 | | |
| Bimane | 398 | 490 |
| Bisbenzamide | 360 | 461 |
| Blancophor FFG | 390 | 470 |
| Blancophor SV | 370 | 435 |
| BOBO ™ -1 | 462 | 481 |
| BOBO ™ -3 | 570 | 602 |
| Bodipy 492/515 | 490 | 515 |
| Bodipy 493/503 | 533 | 549 |
| Bodipy 500/510 | 509 | 515 |
| Bodipy 505/515 | 502 | 510 |
| Bodipy 530/550 | 528 | 547 |
| Bodipy 542/563 | 543 | 563 |
| Bodipy 558/568 | 558 | 569 |
| Bodipy 564/570 | 564 | 570 |
| Bodipy 576/589 | 579 | 590 |
| Bodipy 581/591 | 584 | 592 |
| Bodipy 630/650-X | 625 | 642 |
| Bodipy 650/665-X | 647 | 665 |
| Bodipy 665/676 | 605 | 676 |
| Bodipy Fl | 504, 505 | 511, 513 |
| Bodipy FL ATP | 505 | 514 |
| Bodipy Fl-Ceramide | 505 | 511 |
| Bodipy R6G SE | 528 | 547 |
| Bodipy TMR | 542 | 574 |
| Bodipy TMR-X conjugate | 544 | 573 |
| Bodipy TMR-X, SE | 544 | 570 |
| Bodipy TR | 589 | 617 |
| Bodipy TR ATP | 591 | 620 |
| Bodipy TR-X SE | 588 | 616 |
| BO-PRO ™ -1 | 462 | 481 |
| BO-PRO ™ -3 | 544 | 570 |
| Brilliant Sulphoflavin FF | 430 | 520 |
| Calcein | 494 | 517 |
| Calcein Blue | 373 | 440 |
| Calcium Crimson ™ | 588, 589 | 611, 615 |
| Calcium Green | 501, 506 | 531 |
| Calcium Green-1 Ca2+ Dye | 506 | 531 |
| Calcium Green-2 Ca2+ | 506/503 | 536 |
| Calcium Green-5N Ca2+ | 506 | 532 |
| Calcium Green-C18 Ca2+ | 509 | 530 |
| Calcium Orange | 549 | 575, 576 |
| Calcofluor White | 385, 395, 405 | 437, 440, 445 |
| Carboxy-X-rhodamine (5-ROX) | 576 | 601 |
| Cascade Blue ™ | 377-399 | 420-423 |
| Cascade Yellow | 399, 400 | 550, 552 |
| Catecholamine | 410 | 470 |
| CFDA | 494 | 520 |
| CFP—Cyan Fluorescent Protein | 430-453 | 474-501 |
| Chlorophyll | 480 | 650 |
| Chromomycin A | 436-460 | 470 |
| Chromomycin A | 445 | 575 |
| CMFDA | 494 | 520 |
| Coelenterazine | (429) | 465 |
| Coelenterazine cp | (430) | 442 |
| Coelenterazine f | (437) | 473 |
| Coelenterazine fcp | | 452 |
| Coelenterazine h | (437) | 464 |
| Coelenterazine hcp | (433) | 444 |
| Coelenterazine ip | (431) | 467 |
| Coelenterazine O | 460 | 575 |
| Coumarin Phalloidin | 387 | 470 |
| CPM Methylcoumarin | 384 | 469 |
| CTC | 400-450 | 602 |
| Cy2 ™ | 489 | 506 |
| Cy3.1 8 | 554 | 568 |
| Cy3.5 ™ | 581 | 598 |
| Cy3 ™ | 514, 552, 554 | 566, 570 |
| Cy5.1 8 | 649 | 666 |
| Cy5.5 ™ | 675 | 695 |
| Cy5 ™ | 649 | 666, 670 |
| Cy7 ™ | 710, 743 | 767, 805 |
| Cyan GFP | 433 (453) | 475 (501) |
| cyclic AMP Fluorosensor (FiCRhR) | 500 | 517 |
| d2 | | |
| Dabcyl | 453 | |
| Dansyl | 340 | 578 |
| Dansyl Amine | 337 | 517 |
| Dansyl Cadaverine | 335 | 518 |
| Dansyl Chloride | 372 | 518 |
| Dansyl DHPE | 336 | 517 |
| Dansyl fluoride | 356 | none |
| DAPI | 359 | 461 |
| Dapoxyl | 403 | 580 |
| Dapoxyl 2 | 374 | 574 |
| Dapoxyl 3 | 373 | 574 |
| DCFDA | 504 | 529 |
| DCFH (Dichlorodihydrofluorescein | 505 | 535 |
| Diacetate) | | |
| DDAO | 463 | 607 |
| DHR (Dihydorhodamine 123) | 505 | 534 |
| Di-4-ANEPPS | 496 | 705 |
| Di-8-ANEPPS (non-ratio) | 488, 498 | 605, 713 |
| DiA (4-Di-16-ASP) | 456 | 591 |
| DIDS | 341 | 415 |
| Dihydorhodamine 123 (DHR) | 505 | 535 |
| DiO (DiOC18(3)) | 484, 487 | 501, 502 |
| DiR | 748 | 780 |
| DiR (DiIC18(7)) | 750 | 779 |
| Dopamine | 340 | 490-520 |
| DsRed | 558 | 583 |
| DTAF | 494 | 520 |
| DY-630-NHS | 621 | 660 |
| DY-635-NHS | 634 | 664 |
| EBFP | 383 | 447 |
| ECFP | 436 | 474 |
| EGFP | 488, 498 | 507, 516 |
| ELF 97 | 345 | 530 |
| Eosin | 524 | 545 |
| Erythrosin | 529, 532 | 554, 555 |
| Erythrosin ITC | 529 | 555 |
| Ethidium homodimer-1 (EthD-1) | 528 | 617 |
| Euchrysin | 430 | 540 |
| Europium (III) chloride | 345 | 614 |
| Europium | 345 | 615-620 |
| EYFP | 513, 520 | 527, 532 |
| Fast Blue | 360 | 440 |
| FDA | 494 | 520 |
| Feulgen (Pararosaniline) | 570 | 625 |
| FITC | 490-494 | 520-525 |
| FL-645 | 615-625 | 665 |
| Flazo Orange | 375-530 | 612 |
| Fluo-3 | 480-506, 506 | 520, 527 |
| Fluo-4 | 494 | 516 |
| Fluorescein Diacetate | 494 | 520 |
| Fluoro-Emerald | 495 | 524 |
| Fluoro-Gold (Hydroxystilbamidine) | 361 | 536 |
| Fluor-Ruby | 555 | 582 |
| FluorX | 494 | 520 |
| FM 1-43 ™ | 479 | 598 |
| FM 4-46 | 515 | 640 |
| Fura Red ™ (high pH) | 572 | 657 |
| Fura-2, high calcium | 335 | 505 |
| Fura-2, low calcium | 363 | 512 |
| Genacryl Brilliant Red B | 520 | 590 |
| Genacryl Brilliant Yellow 10GF | 430 | 485 |
| Genacryl Pink 3G | 470 | 583 |
| Genacryl Yellow 5GF | 430 | 475 |
| GFP (S65T) | 498 | 516 |
| GFP red shifted (rsGFP) | 498 | 516 |
| GFP wild type, non-UV excitation | 475 | 509 |
| (wtGFP) | | |
| GFP wild type, UV excitation (wtGFP) | 395 | 509 |
| GFPuv | 385 | 508 |
| Gloxalic Acid | 405 | 460 |
| Granular Blue | 355 | 425 |
| Haematoporphyrin | 530-560 | 580 |
| Hoechst 33258 | 347 | 483 |
| Hoechst 33342 | 347 | 483 |
| Hoechst 34580 | 392 | 440 |
| HPTS | 355 | 465 |
| Hydroxycoumarin | 325-360 | 386-455 |
| Hydroxystilbamidine (FluoroGold) | 361 | 536 |
| Hydroxytryptamine | 400 | 530 |
| Indodicarbocyanine (DiD) | 644 | 665 |
| Indotricarbocyanine (DiR) | 748 | 780 |
| Intrawhite Cf | 360 | 430 |
| JC-1 | 514 | 529 |
| JO-JO-1 | 532 | 544 |
| JO-PRO-1 | 532 | 544 |
| LaserPro | 795 | 812 |
| Laurodan | 355 | 460 |
| LDS 751 | 543, 590 | 712, 607 |
| Leucophor PAF | 370 | 430 |
| Leucophor SF | 380 | 465 |
| Leucophor WS | 395 | 465 |
| Lissamine Rhodamine | 572, 577 | 591, 592 |
| Lissamine Rhodamine B | 577 | 592 |
| LOLO-1 | 566 | 580 |
| LO-PRO-1 | 568 | 581 |
| Lucifer Yellow | 425, 428 | 528, 536, 540 |
| Mag Green | 507 | 531 |
| Magdala Red (Phloxin B) | 524 | 600 |
| Magnesium Green | 506, 507 | 531 |
| Magnesium Orange | 550 | 575 |
| Malachite Green | 628 | |
| Marina Blue | 362 | 459 |
| Maxilon Brilliant Flavin 10 GFF | 450 | 495 |
| Maxilon Brilliant Flavin 8 GFF | 460 | 495 |
| Merocyanin | 555 | 578 |
| Methoxycoumarin | 360 | 410 |
| Mitotracker Green FM | 551 | 576 |
| Mitotracker Orange | 551 | 576 |
| Mitotracker Red | 578 | 599 |
| Mitramycin | 450 | 470 |
| Monobromobimane | 398 | 490 |
| Monobromobimane (mBBr-GSH) | 398 | 500 |
| Monochlorobimane | 390 | 461 |
| MPS (Methyl Green Pyronine Stilbene) | 364 | 395 |
| NBD | 466 | 539 |
| NBD Amine | 450 | 530 |
| Nile Red | 515-555, 559 | 590, 640 |
| Nitrobenzoxadidole | 465 | 510-650 |
| Noradrenaline | 340 | 490-520 |
| Nuclear Fast Red | 289-530 | 580 |
| Nuclear Yellow | 365 | 495 |
| Nylosan Brilliant Iavin E8G | 460 | 510 |
| Oregon Green ™ | 503 | 522 |
| Oregon Green 488-X | 494 | 517 |
| Oregon Green ™ 488 | 490, 493 | 514, 520 |
| Oregon Green ™ 500 | 497 | 517 |
| Oregon Green ™ 514 | 506 | 526 |
| Pacific Blue | 405 | 455 |
| Pararosaniline (Feulgen) | 570 | 625 |
| PE-Cy5 | 488 | 670 |
| PE-Cy7 | 488 | 755, 767 |
| PerCP | 488 | 675 |
| PerCP-Cy5.5 | 488 | 710 |
| PE-TexasRed (Red 613) | 488 | 613 |
| Phloxin B (Magdala Red) | 524 | 600 |
| Phorwite AR | 360 | 430 |
| Phorwite BKL | 370 | 430 |
| Phorwite Rev | 380 | 430 |
| Phorwite RPA | 375 | 430 |
| Phosphine 3R | 465 | 565 |
| PhotoResist | 365 | 610 |
| Phycoerythrin B [PE] | 546-565 | 575 |
| Phycoerythrin R [PE] | 565 | 578 |
| PKH26 | 551 | 567 |
| PKH67 | 496 | 520 |
| PMIA | 341 | 376 |
| Pontochrome Blue Black | 535-553 | 605 |
| POPO-1 | 433 | 457 |
| POPO-3 | 533 | 574 |
| PO-PRO-1 | 539 | 567 |
| PO-PRO-3 | 539 | 567 |
| Primuline | 410 | 550 |
| Procion Yellow | 470 | 600 |
| Propidium Iodid (PI) | (305), 536, 538 | 617 |
| PyMPO | 412, 415 | 561, 564, 570 |
| Pyrene | 360 | 387 |
| Pyronine | 410 | 540 |
| Pyronine B | 540-590 | 560-650 |
| Pyrozal Brilliant Flavin 7GF | 365 | 495 |
| QSY 7 | 560 | |
| Quinacrine Mustard | 440 | 510 |
| Resorufin | 571 | 584, 585 |
| RH 414 | 532 | 716 |
| Rhod-2 | 552 | 576 |
| Rhodamine | 550 | 573 |
| Rhodamine 110 | 496, 497 | 520 |
| Rhodamine 123 | 507 | 529 |
| Rhodamine 5 GLD | 470 | 565 |
| Rhodamine 6G | 525 | 555 |
| Rhodamine B 540 | 523-557 | 595 |
| Rhodamine B 200 | 523-557 | 595 |
| Rhodamine B extra | 550 | 605 |
| Rhodamine BB | 540 | 580 |
| Rhodamine BG | 540 | 572 |
| Rhodamine Green | 502 | 527 |
| Rhodamine Phallicidine | 558, 542 | 575, 565 |
| Rhodamine Phalloidine | 542 | 565 |
| Rhodamine Red | 570 | 590 |
| Rhodamine WT | 530 | 555 |
| Rose Bengal | 525, 540 | 550-600 |
| R-phycoerythrin (PE) | 565 | 578 |
| red shifted GFP (rsGFP, S65T) | 473 | 509 |
| S65A | 471 | 504 |
| S65C | 479 | 507 |
| S65L | 484 | 510 |
| S65T | 488 | 511 |
| Sapphire GFP | 395 | 511 |
| Serotonin | 365 | 520-540 |
| Sevron Brilliant Red 2B | 520 | 595 |
| Sevron Brilliant Red 4G | 500 | 583 |
| Sevron Brilliant Red B | 530 | 590 |
| Sevron Orange | 440 | 530 |
| Sevron Yellow L | 430 | 490 |
| sgBFP ™ | 387 | 450 |
| sgBFP ™ (super glow BFP) | 387 | 450 |
| sgGFP ™ | 474 | 488 |
| sgGFP ™ (super glow GFP) | 474 | 509 |
| SITS | 336 | 436 |
| SITS (Primuline) | 395-425 | 450 |
| SITS (Stilbene Isothiosulphonic Acid) | 365 | 460 |
| SPQ (6-methoxy-N-(3-sulfopropyl)- | 344 | 443 |
| quinolinium) | | |
| Stilbene | 335 | 440 |
| Sulphorhodamine B can C | 520 | 595 |
| Sulphorhodamine G Extra | 470 | 570 |
| Tetracycline | 390-425 | 525-560 |
| Tetramethylrhodamine | 555 | 576 |
| Texas Red ™ | 595 | 620 |
| Texas Red-X ™ conjugate | 595 | 615 |
| Thiadicarbocyanine (DiSC3) | 651, 653 | 674, 675 |
| Thiazine Red R | 596 | 615 |
| Thiazole Orange | 510 | 530 |
| Thioflavin 5 | 430 | 550 |
| Thioflavin S | 430 | 550 |
| Thioflavin TCN | 350 | 460 |
| Thiolyte | 370-385 | 477-488 |
| Thiozole Orange | 453 | 480 |
| Tinopol CBS (Calcofluor White) | 390 | 480 |
| TMR | 550 | 573 |
| TO-PRO-1 | 515 | 531 |
| TO-PRO-3 | 644 | 657 |
| TO-PRO-5 | 747 | 770 |
| TOTO-1 | 514 | 531, 533 |
| TOTO-3 | 642 | 660 |
| TriColor (PE-Cy5) | (488) 650 | 667 |
| TRITC | 550 | 573 |
| (TetramethylRodamineIsoThioCyanate) | | |
| True Blue | 365 | 425 |
| TruRed | 490 | 695 |
| Ultralite | 656 | 678 |
| Uranine B | 420 | 520 |
| Uvitex SFC | 365 | 435 |
| wt GFP | 395 (475) | 508 |
| WW 781 | 605 | 639 |
| XL665 | | |
| X-Rhodamine | 580 | 601 |
| XRITC | 582 | 601 |
| Xylene Orange | 546 | 580 |
| Y66F | 360 | 508 |
| Y66H | 362 | 442 |
| Y66W | 436 | 485 |
| Yellow GFP | 513 | 527 |
| YFP | 513, 520 | 527, 532 |
| YO-PRO-1 | 491 | 506 |
| YO-PRO-3 | 613 | 629 |
| YOYO-1 | 491 | 508, 509 |
| YOYO-3 | 612 | 631 |

In some embodiments, the FRET donor and the FRET acceptor are chosen based on one or more, including all, of the following: (1) the emission spectrum of the FRET donor should overlap with the excitation spectrum of the FRET acceptor; (2) The emission spectra of the FRET partners (i.e., the FRET donor and the FRET acceptor) should show non-overlapping fluorescence; (3) the FRET quantum yield (i.e., the energy transferred from the FRET donor to the FRET acceptor) should be as high as possible (for example, FRET should have about a 1-100%, e.g., a 10%, 20%, 30%, 40%, 50%, 60%, 70%, 80%, 85%, 90%, 95%, 98%, and 99% efficiency over a measured distance of 1-20 nm, e.g., 5-10 nm); (4) the FRET signal (i.e., fluorescence) must be distinguishable from fluorescence produced by the sample, e.g., auto fluorescence; and (5) the FRET donor and the FRET acceptor should have half lives that facilitate detection of the FRET signal (e.g., FRET can be bright and can occur on a timescale ranging from 10⁻⁹ seconds to 10+ seconds).

In some embodiments, the FRET donor and the FRET acceptor are selected independently from the group of fluorophores listed in Table 1.

In some embodiments, the FRET donor is Oregon Green 488 (OG-488).

In some embodiments, the FRET acceptor is Alexa 546.

In some embodiments, the detection is by a TR-FRET assay technique. TR-FRET is a combination of time-resolved fluorescence (TRF) and FRET. TRF reduces background fluorescence by delaying reading the fluorescent signal, for example, by about 10 nano seconds. Following this delay (i.e., the gating period), the longer lasting fluorescence in the sample is measured. Thus, using TR-FRET, interfering background fluorescence, that may for example be due to interfering substances in the sample, is not co-detected, but rather, only the fluorescence generated or suppressed by the energy transfer is measured. The resulting fluorescence of the TR-FRET system is determined by means of appropriate measuring devices. Such time-resolved detection systems use, for example, pulsed laser diodes, light emitting diodes (LEDs) or pulsed dye lasers as the excitation light source. The measurement occurs after an appropriate time delay, i.e. after the interfering background signals have decayed. Devices and methods for determining time-resolved FRET signals are described in the art.

TR-FRET assay requires that the signal of interest must correspond to a compound with a long fluorescent lifetime. Such long-lived fluorescent compounds are the rare earth lanthanides. For example, Eu³⁺ has a fluorescent lifetime in the order of milliseconds. Accordingly, in some embodiments, the TR-FRET donor can be a lanthanide. A lanthanide can be selected from the group consisting of europium (Eu); terbium (Tb); samarium; second generation and functional homologues of Eu, Tb, and samarium; and any combinations thereof. As used herein, Eu includes Eu and all Eu homologues, e.g., Eu³⁺. In some embodiments, the TR-FRET donor can be DsRed or Ri2. It is to be understood that selection of the appropriate TR-FRET donor requires consideration of the above listed criteria for FRET donor and the specific TR-FRET acceptor selected.

A TR-FRET acceptor can be selected from the group consisting of fluorescein, Cy5, allophycocyanin (APC—e.g., XL665, d2, and BG-647), and fluorescent protein (e.g., GFP, CFP, YFP, BFP, and RFP).

In some embodiments, the TR-FRET donor can be terbium and the TR-FRET acceptor can be fluorescein. In some embodiments, the TR-FRET donor can be Eu and the TR-FRET acceptor can be Cy5 or APC (e.g., XL665, d2, and BG-647).

Without wishing to be bound by a theory, the TR-FRET donor and the TR-FRET acceptor can be combined with a second compound that enhances the function of the TR-FRET donor and/or the TR-FRET acceptor. For example, the TR-FRET donor and the TR-FRET acceptor can be combined with cryptate encapsulation to extend the half-life of the fluorophore. Alternatively, or in addition, the TR-FRET donor the TR-FRET acceptor can be combined with, e.g., DELFIA® enhancement system. In some embodiments, the TR-FRET donor and the TR-FRET acceptor can be combined with, for example buffers, salts, enhancers, chelators, and stabilizers (e.g., photo-stabilizers) that enhance or extend the life or detection of the TR-FRET signal.

When the detection is by a FRET based assay, two or more components of the complex are labeled with fluorescent molecules (FRET donor and acceptor) having the proper emission and excitation spectra. One of skill will appreciate that the screening assays described herein can be practiced by labeling two or more components of the complex with any combination of suitable FRET acceptor and donor. In some embodiments, two components of the complex are labeled with suitable FRET acceptor and donor.

In some embodiments, the NFAT peptide and the oligonucleotide are labeled for FRET detection. In some embodiments, the NFAT peptide comprises the FRET donor and the oligonucleotide comprises the FRET acceptor. In some other embodiment, the NFAT peptide comprises the FRET acceptor and the oligonucleotide comprises the FRET donor.

In some embodiments, the NFAT peptide and at least one of the Fos peptide or the Jun peptide are labeled for FRET detection. In some embodiments, the NFAT peptide comprises the FRET donor and at least one of the Fos or Jun peptide comprises the FRET acceptor. In some other embodiment, the NFAT peptide comprises the FRET acceptor and at least one of the Fos or Jun peptide comprises the FRET donor.

In some embodiments, the oligonucleotide and at least one of the Fos or Jun peptide are labeled for FRET. In some further embodiments of this, the oligonucleotide comprises the FRET donor and at least one of the Fos or Jun peptide comprises the FRET acceptor. In some other further embodiment, the oligonucleotide comprises the FRET acceptor and at least one of the Fos or Jun peptide comprises the FRET donor.

In one embodiment, the oligonucleotide comprises the FRET acceptor and the Fos peptide comprises the FRET donor.

In some embodiments, the screening assay is a high-throughput screening assay. HTS is a relative term, but is generally defined as the testing of 10,000 to 100,000 compounds per day, accomplished with mechanization that ranges from manually operated workstations to fully automated robotic systems using robotics, data processing and control software, liquid handling devices, and sensitive detectors. HTS screening techniques generally provide advantages over non-HTS methods as they are faster, due to automation, highly reproducible, and cost effective. HTS allows a researcher to quickly conduct millions of biochemical, genetic or pharmacological tests. Accordingly, HTS allows large numbers of samples to be screened and/or validated per day. HTS can considerably reduce the cost of drug discovery and quality control. High-Throughput Screening techniques are well known to one skilled in the art, for example, those described in U.S. Pat. Nos. 5,976,813; 6,472,144; 6,692,856; 6,824,982; and 7,091,048, and contents of each of which is herein incorporated by reference in its entirety.

HTS uses automation to run a screen or an assay against a library of candidate compounds. An assay is a test for specific activity: usually inhibition or stimulation of a biochemical or biological mechanism. Typical HTS screening libraries or “decks” can contain from 100,000 to more than 2,000,000 compounds.

The key labware or testing vessel of HTS is the microtiter plate: a small container, usually disposable and made of plastic, which features a grid of small, open divots called wells. Modern microplates for HTS generally have either 96, 384, 1536, or 3456 wells. These are all multiples of 96, reflecting the original 96 well microplate with 8×12 9 mm spaced wells.

To prepare for an assay, the researcher fills each well of the plate with the appropriate reagents that he or she wishes to conduct the experiment with, such as a components of the complex and the test compound. After some incubation time has passed measurements are taken across all the plate's wells, either manually or by a machine. Manual measurements are often necessary when the researcher is using microscopy to (for example) seek changes that a computer could not easily determine by itself. Otherwise, a specialized automated analysis machine can run a number of experiments on the wells such as colorimetric measurements, fluorescent measurements, radioactivity counting, etc. In this case, the machine outputs the result of each experiment as a grid of numeric values, with each number mapping to the value obtained from a single well. A high-capacity analysis machine can measure dozens of plates in the space of a few minutes like this, generating thousands of experimental data points very quickly.

As used herein, the term “test compound” refers to compounds and/or compositions that are to be screened for their ability to modulate the NFAT:transcription factor interaction. The test compounds can include a wide variety of different compounds, including chemical compounds and mixtures of chemical compounds, e.g., small or larger organic or inorganic molecules; saccharides; oligosaccharides; polysaccharides; biological macromolecules, e.g., peptides, proteins, and peptide analogs and derivatives; peptidomimetics; nucleic acids; nucleic acid analogs and derivatives; an extract made from biological materials such as bacteria, plants, fungi, or animal cells; animal tissues; naturally occurring or synthetic compositions; and any combinations thereof. In some embodiments, the test compound is a small molecule.

As used herein, the term “small molecule” can refer to compounds that are “natural product-like,” however, the term “small molecule” is not limited to “natural product-like” compounds. Rather, a small molecule is typically characterized in that it contains several carbon-carbon bonds, and has a molecular weight of less than 5000 Daltons (5 kD), preferably less than 3 kD, still more preferably less than 2 kD, and most preferably less than 1 kD. In some cases it is preferred that a small molecule have a molecular weight equal to or less than 700 Daltons. Examples of “small molecules” that occur in nature include, but are not limited to, taxol, dynemicity and rapamycin, Examples of “small molecules” that are synthesized in the laboratory include, but are not limited to, compounds described in Tan et al., (“Stereoselective Synthesis of over Two Million Compounds Having Structural Features Both Reminiscent of Natural Products and Compatible with Miniaturized Cell-Based Assays” J. Am. Chem. Soc. 1998, 120, 8565; incorporated herein by reference).

As used herein, the term “peptide” is used in its broadest sense to refer to compounds containing two or more amino acids, amino acid equivalents or other non-amino groups joined to each other by peptide bonds or modified peptide bonds. Peptide equivalents can differ from conventional peptides by the replacement of one or more amino acids with related organic acids (such as PABA), amino acids or the like or the substitution or modification of side chains or functional groups. A peptide can be of any size so long; however, in some embodiments, peptides having twenty or fewer total amino acids are preferred. Additionally, the peptide can be linear or cyclic. Peptide sequences specifically recited herein are written with the amino terminus on the left and the carboxy terminus on the right. In addition, the term “peptide” broadly includes proteins, which generally are polypeptides. As used herein, the term “protein” is used to describe proteins as well as fragments thereof. Thus, any chain of amino acids that exhibits a three dimensional structure is included in the term “protein”, and protein fragments are accordingly embraced. In some embodiments, a peptide is from 10 to about 100 amino acids in length.

A peptidomimetic is a molecule capable of folding into a defined three-dimensional structure similar to a natural peptide.

As used herein, the term “nucleic acid” or “oligonucleotide” or grammatical equivalents herein means at least two nucleotides, including analogs or derivatives thereof, that are covalently linked together. The nucleic acids can be single stranded or double stranded. The nucleic acid can be DNA, RNA or a hybrid, where the nucleic acid contains any combination of deoxyribo- and ribo-nucleotides, and any combination of uracil, adenine, thymine, cytosine and guanine. The nucleic acids can comprise one or more backbone modifications, e.g., phosphoramide (Beaucage et al., Tetrahedron 49(10):1925 (1993) and references therein; Letsinger, J. Org. Chem. 35:3800 (1970)), phosphorothioate, phosphorodithioate, O-methylphosphoroamidite linkages (see Eckstein, Oligonucleotides and Analogues: A Practical Approach, Oxford University Press), or peptide nucleic acid linkages (see Egholm, J. Am. Chem. Soc. 114:1895 (1992); Meier et al., Chem. Int. Ed. Engl. 31:1008 (1992); Nielsen, Nature, 365:566 (1993)). The nucleic acids can also include modifications to nucleobase and/or sugar moieties of nucleotides. Exemplary sugar modifications at the sugar moiety include replacement of 2′-OH with halogens (e.g., fluoro), O-methyl, O-methoxyethyl, NH₂, SH and S-methyl. In some embodiments, an oligonucleotide can be from 10 to about 100 nucleotides in length, e.g., from 10 to 75, from 10 to 50, from 10 to 25, or from 10 to 20 nucleotides in length.

As used herein, the term “saccharide” refers to mono-, di- and, tri-saccharides. An “oligosaccharide” refers without limitation to several (e.g., four to ten) covalently linked monosaccharide units. A “polysaccharide” refers without limitation to many (e.g., eleven or more) covalently linked sugar units. Polysaccharides can have molecular masses ranging well into millions of Daltons. Exemplary oligosaccharides and polysaccharides include, but are not limited to, fructooligosaccharide, galactooligosaccharides, mannanoligosaccharides, glycogen, starch (amylase, amylopectin), glycosaminoglycans (e.g., hyaluronic acid, chondroitin-4-sulfate, chondroitin-6-sulfate, dermatan sulfate, keratin sulfate, heparin and the like), cellulose, beta-glucan (zymosan, lentinan, sizofiran), maltodextrin, inulin, levan beta (2->6), chitin, and chitosan.

The number of possible test compounds runs into millions. Methods for developing small molecule, polymeric and genome based libraries are described, for example, in Ding, et al. J. Am. Chem. Soc. 124: 1594-1596 (2002); Zuckermann, et al., J. Med. Chem. 37: 2678-85 (1994); Lynn, et al., J. Am. Chem. Soc. 123: 8155-8156 (2001); and Lam (1997) Anticancer Drug Des. 12:145 (1997); DeWitt et al. (1993) Proc. Natl. Acad. Sci. U.S.A. 90:6909; Erb et al. (1994) Proc. Natl. Acad. Sci. USA 91:11422; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233; Houghten (1992) Biotechniques 13:412-421; Fodor (1993) Nature 364:555-556; Cull et al. (1992) Proc Natl Acad Sci USA 89:1865-1869; Scott and Smith (1990) Science 249:386-390; Devlin (1990) Science 249:404-406; Cwirla et al. (1990) Proc. Natl. Acad. Sci. 87:6378-6382; and Felici (1991) J. Mol. Biol. 222:301310, and U.S. Pat. No. 5,223,409, content of all of which is incorporated herein by reference. Commercially available compound libraries can be obtained from, e.g., ArQule, Pharmacopia, Graffinity, Panvera, Vitas-M Lab, Biomol International and Oxford. These libraries can be screened using the screening devices and methods described herein. Chemical compound libraries such as those from NIH Roadmap, Molecular Libraries Screening Centers Network (MLSCN) can also be used. A comprehensive list of compound libraries can be found at www.broad.harvard.edu/chembio/platform/screening/compound_libraries/index.htm. A chemical library or compound library is a collection of stored chemicals usually used ultimately in high-throughput screening or industrial manufacture. The chemical library can consist in simple terms of a series of stored chemicals. Each chemical has associated information stored in some kind of database with information such as the chemical structure, purity, quantity, and physiochemical characteristics of the compound.

Depending upon the particular embodiment being practiced, the test compounds can be provided free in solution, or may be attached to a carrier, or a solid support, e.g., beads. A number of suitable solid supports may be employed for immobilization of the test compounds. Examples of suitable solid supports include agarose, cellulose, dextran (commercially available as, i.e., Sephadex, Sepharose) carboxymethyl cellulose, polystyrene, polyethylene glycol (PEG), filter paper, nitrocellulose, ion exchange resins, plastic films, polyaminemethylvinylether maleic acid copolymer, glass beads, amino acid copolymer, ethylene-maleic acid copolymer, nylon, silk, etc. Additionally, for the methods described herein, test compounds may be screened individually, or in groups. Group screening is particularly useful where hit rates for effective test compounds are expected to be low such that one would not expect more than one positive result for a given group.

In some embodiments, the test compound inhibits formation of a NFAT:Fos:Jun:DNA complex by at least 5%, at least 10%, at least 20%, at least 30%, at least 40%, at least 50%, at least 50%, at least 70%, at least 80%, at least 90%, or 100% (complete inhibition) relative to control or reference. In some embodiments the test compound does not completely (i.e., 100% inhibition) inhibit formation of a NFAT:Fos:Jun:DNA complex.

The peptides of the invention can be synthesized according to the usual methods of solution and solid phase peptide chemistry, or by classical methods known in the art. Purification of peptides is well known in the art and can be, for example, HPLC. Methods describing useful peptide synthesis and purification methods can be found, for example, in U.S. Pat. App. Pub. No. 20060084607, content of which is incorporated herein by reference.

Peptides described herein can be synthetically constructed by suitable known peptide polymerization techniques, such as exclusively solid phase techniques, partial solid-phase techniques, fragment condensation or classical solution couplings. For example, the peptides of the invention can be synthesized by the solid phase method using standard methods based on either t-butyloxycarbonyl (BOC) or 9-fluorenylmethoxy-carbonyl (FMOC) protecting groups. This methodology is described by G. B. Fields et al. in Synthetic Peptides: A User's Guide, W. M. Freeman & Company, New York, N.Y., pp. 77-183 (1992) and in the textbook “Solid-Phase Synthesis”, Stewart & Young, Freemen & Company, San Francisco, 1969, and are exemplified by the disclosure of U.S. Pat. No. 4,105,603, issued Aug. 8, 1979. Classical solution synthesis is described in detail in “Methoden der Organischen Chemic (Houben-Weyl): Synthese von Peptiden”, E. Wunsch (editor) (1974) Georg Thieme Verlag, Stuttgart West Germany. The fragment condensation method of synthesis is exemplified in U.S. Pat. No. 3,972,859. Other available syntheses are exemplified in U.S. Pat. No. 3,842,067, U.S. Pat. No. 3,872,925, issued Jan. 28, 1975, Merrifield B, Protein Science (1996), 5: 1947-1951; The chemical synthesis of proteins; Mutter M, Int J Pept Protein Res 1979 March; 13 (3): 274-7 Studies on the coupling rates in liquid-phase peptide synthesis using competition experiments; and Solid Phase Peptide Synthesis in the series Methods in Enzymology (Fields, G. B. (1997) Solid-Phase Peptide Synthesis. Academic Press, San Diego.#9830). Content of all of the foregoing disclosures is incorporated herein by reference.

The peptides of the invention can also be expressed in and purified from a cell. A peptide can be expressed from an endogenous gene in a cell or alternatively expressed from a an exogenous gene transfected into a cell, e.g., a bacterial cell.

In some embodiments, a peptide described herein comprises a D-amino acid, a beta-amino-acid, a gamma-amino acid, a chemically modified amino acid, a modified amide linkage, or any combinations thereof. As used herein, the term “chemically modified amino acid” refers to an amino acid that has been treated with one or more reagents. As used herein, the term “modified amide linkage” refers to an amide bond in the backbone of the peptide which is replaced by a linkage selected from the group consisting of reduced psi peptide bond, urea, thiourea, carbamate, sulfonyl urea, trifluoroethylamine, ortho-(aminoalkyl)-phenylacetic acid, para-(aminoalkyl)-phenylacetic acid, meta-(aminoalkyl)-phenylacetic acid, thioamide, tetrazole, boronic ester, and olefinic group.

In some embodiments, a peptide, e.g. NFAT peptide, Fos peptide, or Jun peptide, comprises at least one amino acid selected from the group consisting of homocysteine, phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine (3-mercapto-D-valine), ornithine, citruline, alpha-methyl-alanine, para-benzoylphenylalanine, para-amino phenylalanine, p-fluorophenylalanine, phenylglycine, propargylglycine, sarcosine, and tert-butylglycine), diaminobutyric acid, 7-hydroxy-tetrahydroisoquinoline carboxylic acid, naphthylalanine, biphenylalanine, cyclohexylalanine, amino-isobutyric acid, norvaline, norleucine, tert-leucine, tetrahydroisoquinoline carboxylic acid, pipecolic acid, phenylglycine, homophenylalanine, cyclohexylglycine, dehydroleucine, 2,2-diethylglycine, 1-amino-1-cyclopentanecarboxylic acid, 1-amino-1-cyclohexanecarboxylic acid, amino-benzoic acid, amino-naphthoic acid, gamma-aminobutyric acid, difluorophenylalanine, nipecotic acid, alpha-amino butyric acid, thienyl-alanine, t-butylglycine, and derivatives thereof.

The oligonucleotides used in accordance with this invention can be synthesized with solid phase synthesis, see for example “Oligonucleotide synthesis, a practical approach”, Ed. M. J. Gait, IRL Press, 1984; “Oligonucleotides and Analogues, A Practical Approach”, Ed. F. Eckstein, IRL Press, 1991 (especially Chapter 1, Modern machine-aided methods of oligodeoxyribonucleotide synthesis, Chapter 2, Oligoribonucleotide synthesis, Chapter 3,2′-O-Methyloligoribonucleotides: synthesis and applications, Chapter 4, Phosphorothioate oligonucleotides, Chapter 5, Synthesis of oligonucleotide phosphorodithioates, Chapter 6, Synthesis of oligo-2′-deoxyribonucleoside methylphosphonates, and. Chapter 7, Oligodeoxynucleotides containing modified bases. Other particularly useful synthetic procedures, reagents, blocking groups and reaction conditions are described in Martin, P., Helv. Chim. Acta, 1995, 78, 486-504; Beaucage, S. L. and Iyer, R. P., Tetrahedron, 1992, 48, 2223-2311 and Beaucage, S. L. and Iyer, R. P., Tetrahedron, 1993, 49, 6123-6194, or references referred to therein. Modification described in WO 00/44895, WO01/75164, or WO02/44321 can be used herein.

Unmodified oligonucleotides can be less than optimal in some applications, e.g., unmodified oligonucleotides can be prone to degradation by e.g., nucleases. However, chemical modifications to one or more of the subunits of oligonucleotide can confer improved properties, e.g., can render oligonucleotides more stable to nucleases. Typical oligonucleotide modifications can include one or more of: (i) alteration, e.g., replacement, of one or both of the non-linking phosphate oxygens and/or of one or more of the linking phosphate oxygens in the phosphodiester intersugar linkage; (ii) alteration, e.g., replacement, of a constituent of the ribose sugar, e.g., of the 2′ hydroxyl on the ribose sugar; (iii) wholesale replacement of the phosphate moiety with “dephospho” linkers; (iv) modification or replacement of a naturally occurring base with a non-natural base; (v) replacement or modification of the ribose-phosphate backbone, e.g. peptide nucleic acid (PNA); (vi) modification of the 3′ end or 5′ end of the oligonucleotide, e.g., removal, modification or replacement of a terminal phosphate group or conjugation of a moiety, e.g., conjugation of a ligand, to either the 3′ or 5′ end of oligonucleotide; and (vii) modification of the sugar, e.g., six membered rings.

Accordingly, in some embodiments, an oligonucleotide described herein at least one modification. In some embodiments, the modification is selected from the group consisting of sugar modifications, phosphothioate intersugar (or internucleoside) linkages, non-phosphodiester intersugar linkages, e.g., complete replacement of the phosphodiester linkage, nucleobase modifications, and any combinations thereof.

The compounds selected by the screening assay described herein have immune suppression activity. Accordingly, in another aspect, the invention provides a compound selected by the screening assay described herein. It is to be understood that analogs, derivatives, isomers, and pharmaceutically acceptable salts of the compounds selected by the screening assays described herein are also claimed herein.

In some embodiments, a compound identified by the screening assay is a compound of formula (I) or formula (II) described herein.

In another aspect, the invention provides a method of inhibiting NFAT:AP-1:DNA interaction in a cell, the method comprising contacting a cell with a compound selected by the screening assay described herein.

In some embodiments, the compound to be contacted with the cell is of formula (I):

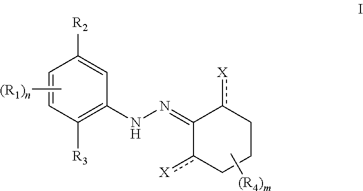

wherein

each R₁, R₂, R₃, and R₄ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; OR^{B}; C(═O)R^{B}; CO₂R^{B}; —C(═O)N(R^{B})₂; —CN; —SCN; —SR^{B}; —SOR^{B}; —SO₂R^{B}; —NO₂; —N(R^{B})₂; —NHC(O)R^{B}; or —C(R^{B})₃; wherein each occurrence of R^{B} is independently hydrogen; halogen; a protecting group; aliphatic; heteroaliphatic; acyl; aryl moiety; heteroaryl; hydroxyl; aloxy; aryloxy; alkylthioxy; arylthioxy; amino; alkylamino; dialkylamino; heteroaryloxy; heteroarylthioxy; alkylhalo;

each X is independently H, O, or R₄,

n is an integer 0-3, inclusive;

m is an integer 0-5, inclusive;

or salt thereof.

In some embodiments, each R₁ is independently hydrogen; halogen; alkyl; OR^{B}; —C(═O)R^{B}; —CO₂R^{B}; —C(═O)N(R^{B})₂; —CN; —SCN; —SR^{B}; —SOR^{B}; —SO₂R^{B}; —NO₂; —N(R^{B})₂; —NHC(O)R^{B}; or —C(R^{B})₃; wherein each occurrence of R^{B} is independently hydrogen; halogen; a protecting group; alkyl; hydroxyl; alkoxy; amino.

In some embodiments, each R₁ is independently hydrogen; halogen; C₁₋₄ alkyl; hydroxyl, or —NO₂. In some embodiments, each R₁ is different. In some embodiments, all R₁ are the same. In some embodiments, at least two R₁ are the same. In some embodiments, at least one R₁ is hydrogen. In some embodiments, all R₁ are hydrogen.

In some embodiments, R₂ is i hydrogen; halogen; alkyl; OR^{B}; —C(═O)R^{B}; —CO₂R^{B}; —C(═O)N(R^{B})₂; —CN; —SCN; —SR^{B}; —SOR^{B}; —SO₂R^{B}; —NO₂; —N(R^{B})₂; —NHC(O)R^{B}; or —C(R^{B})₃; wherein each occurrence of R^{B} is independently hydrogen; halogen; a protecting group; alkyl; hydroxyl; alkoxy; amino.

In some embodiments, R₂ is hydrogen; halogen; C₁₋₄ alkyl; hydroxyl, or —NO₂. In some embodiments, R² is —C(R^{B})₃; wherein each R^{B} is independently hydrogen or halogen. In some embodiments, each R^{B} is halogen. In some embodiments, each R^{B} is F.

In some embodiments R₃ is hydrogen; halogen; alkyl; OR^{B}; —C(═O)R^{B}; —CO₂R^{B}; —C(═O)N(R^{B})₂; —CN; —SCN; —SR^{B}; —SOR^{B}; —SO₂R^{B}; —NO₂; —N(R^{B})₂; —NHC(O)R^{B}; or —C(R^{B})₃; wherein each occurrence of R^{B} is independently hydrogen; halogen; a protecting group; alkyl; hydroxyl; alkoxy; amino, alkylhalo. In some embodiments, R₃ is OR^{B}. In some embodiments, R^{B} is alkylhalo. In some embodiments, alkylhalo is CH₂CF₃.

In some embodiments, each R₄ is independently hydrogen; halogen; alkyl; OR^{B}; —C(═O)R^{B}; —CO₂R^{B}; —C(═O)N(R^{B})₂; —CN; —SCN; —SR^{B}; —SOR^{B}; —SO₂R^{B}; —NO₂; —N(R^{B})₂; —NHC(O)R^{B}; or —C(R^{B})₃; wherein each occurrence of R^{B} is independently hydrogen; halogen; a protecting group; alkyl; hydroxyl; alkoxy; amino.

In some embodiments, each R₄ is independently hydrogen; halogen; C₁₋₄ alkyl; hydroxyl, or —NO₂. In some embodiments, each R₄ is different. In some embodiments, all R₁ are the same. In some embodiments, at least two R₄ are the same. In some embodiments, at least one R₁ is hydrogen. In some embodiments, all R₄ are hydrogen.

In some embodiments, m is 0. In some embodiments, m is 1. In some embodiments, m is 2. In some embodiments, m is 3.

In some embodiments, n is 0. In some embodiments, n is 1. In some embodiments, n is 2. In some embodiments, n is 3. In some embodiments, n is 4. In some embodiments, n is 5.

In some embodiments, n and m are the same. In some embodiments, n and m are different. In some embodiments, at least n or m is 0. In some embodiments, at least n or m is 1.

In some embodiments, n and m are both 0. In some embodiments, n and m are both 1.

In some embodiments, each X is independently H, O, or R₄. In some embodiments, each X is different. In some embodiments, both X are the same. In some embodiments, at least one X is O. In some embodiments, both X are O.

In one embodiment, a compound of formula (I) is

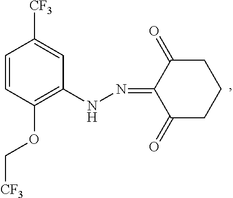 [SMILES: O=C1CCCC(=O)C1=NNc1cc(C(F)(F)F)ccc1OCC(F)(F)F]

also referred to as 1668 P02 herein.

In some embodiments, the compound to be contacted with the cell is of formula (II):

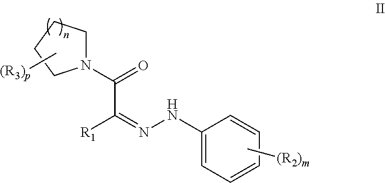

wherein

each R₁, R₂, and R₃, is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; OR^{B}; —C(═O)R^{B}; —CO₂R^{B}; —C(═O)N(R^{B})₂; —CN; —SCN; —SR^{B}; —SOR^{B}; —SO₂R^{B}; —NO₂; —N(R^{B})₂; —NHC(O)R^{B}; or —C(R^{B})₃; wherein each occurrence of R^{B} is independently hydrogen; halogen; a protecting group; aliphatic; heteroaliphatic; acyl; aryl moiety; heteroaryl; hydroxyl; aloxy; aryloxy; alkylthioxy; arylthioxy; amino; alkylamino; dialkylamino; heteroaryloxy; heteroarylthioxy; alkylhalo; and two R₂ can be taken together to form a five or six membered ring;

n is an integer 1-3, inclusive;

m is an integer 0-5, inclusive;

p is an integer 0-7, inclusive;

or salt thereof.

In some embodiments, R₁ is hydrogen; halogen; alkyl; OR^{B}; —C(═O)R^{B}; —CO₂R^{B}; —C(═O)N(R^{B})₂; —CN; —SCN; —SR^{B}; —SOR^{B}; —SO₂R^{B}; —NO₂; —N(R^{B})₂; —NHC(O)R^{B}; or —C(R^{B})₃; wherein each occurrence of R^{B} is independently hydrogen; halogen; a protecting group; alkyl; hydroxyl; alkoxy; amino.

In some embodiments, R₁ is hydrogen; halogen; C₁₋₄ alkyl; hydroxyl, —NO₂, or —CN. In some embodiments, R₁ is CN.

In some embodiments, each R₂ is independently hydrogen; halogen; alkyl; OR^{B}; —C(═O)R^{B}; —CO₂R^{B}; —C(═O)N(R^{B})₂; —CN; —SCN; —SR^{B}; —SOR^{B}; —SO₂R^{B}; —NO₂; —N(R^{B})₂; —NHC(O)R^{B}; or —C(R^{B})₃; wherein each occurrence of R^{B} is independently hydrogen; halogen; a protecting group; alkyl; hydroxyl; alkoxy; amino.

In some embodiments, each R₂ is independently hydrogen; halogen; C₁₋₄ alkyl; hydroxyl, or —NO₂. In some embodiments, each R₂ is different. In some embodiments, all R₂ are the same. In some embodiments, at least two R₂ are the same. In some embodiments, at least one R₂ is hydrogen. In some embodiments, all R₂ are hydrogen.

In some embodiments at least two R₂ are OR^{B}. In some embodiments, the one OR^{B} is at the ortho and one OR^{B} is at the para positions. In some embodiments, the two OR^{B} are linked to form a five membered acetal.

In some embodiments, each R₃ is independently hydrogen; halogen; alkyl; OR^{B}; —C(═O)R^{B}; —CO₂R^{B}; —C(═O)N(R^{B})₂; —CN; —SCN; —SR^{B}; —SOR^{B}; —SO₂R^{B}; —NO₂; —N(R^{B})₂; —NHC(O)R^{B}; or —C(R^{B})₃; wherein each occurrence of R^{B} is independently hydrogen; halogen; a protecting group; alkyl; hydroxyl; alkoxy; amino.

In some embodiments, each R₃ is independently hydrogen; halogen; C₁₋₄ alkyl; hydroxyl, or —NO₂. In some embodiments, each R₃ is different. In some embodiments, all R₃ are the same. In some embodiments, at least two R₃ are the same. In some embodiments, at least one R₃ is hydrogen. In some embodiments, all R₃ are hydrogen.

In some embodiments, n is 1. In some embodiments, n is 2. In some embodiments, n is 3.

In some embodiments, m is 0. In some embodiments, m is 1. In some embodiments, m is 2. In some embodiments, m is 3. In some embodiments, m is 4. In some embodiments, m is 5.

In some embodiments, p is 0. In some embodiments, p is 1. In some embodiments, p is 2. In some embodiments, p is 3. In some embodiments, p is 4. In some embodiments, p is 5. In some embodiments, p is 6. In some embodiments, p is 7.

In some embodiments, n is 1, and both m and p are 0.

In one embodiment, a compound of formula (II) is

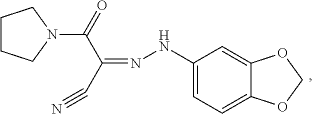 [SMILES: N#C/C(=N/Nc1ccc2c(c1)OCO2)C(=O)N1CCCC1]

also referred to as 1661 N20 herein.

A cell can be contacted with a compound described herein in a cell culture e.g., in vitro or ex vivo, or the compound can be administrated to a subject, e.g., in vivo. In some embodiments of the invention, a compound described herein can be administrated to a subject to treat, and/or prevent a disorder which is characterized by an elevated immune response, e.g., an inflammation causing disease or disorder, cancer, complications after a tissue/organ transplant.

The term “ex vivo” refers to cells which are removed from a living organism and cultured outside the organism (e.g., in a test tube).

The term “contacting” or “contact” as used herein in connection with contacting a population of pancreatic cells includes subjecting the pancreatic cells to an appropriate culture media which comprises the indicated compound. Where the cell is in vivo, “contacting” or “contact” includes administering the compound in a pharmaceutical composition to a subject via an appropriate administration route such that the compound or agent contacts the pancreatic cell population in vivo.

For in vivo methods, a therapeutically effective amount of a compound described herein can be administered to a subject. Methods of administering compounds to a subject are known in the art and easily available to one of skill in the art.

Inhibiting NFAT:AP-1:DNA interactions in a subject can lead to treatment, prevention, amelioration of one or more of diseases or disorders which are characterized by elevated immune response.

Without wishing to be bound by theory any suitable cell culture media can be used for ex vivo methods of the invention.

Without wishing to be bound by a theory, inhibitors of NFAT:AP-1 interactions identified by the screening assay can inhibit immune system activation in a subject. Accordingly, in another aspect, the invention provides, a method of inhibiting an immune response in a subject, the method comprising administering a therapeutically effective amount of a compound described herein to a subject in need thereof.

Without wishing to be bound by a theory, inhibiting the immune system can be useful in situations wherein an elevated immune response is a problem, e.g., an inflammation causing disease or disorder, cancer, complications after a tissue/organ transplant. Inhibiting the immune response can lead to treatment of such diseases and disorders because the compounds described herein can inhibit immune activation, i.e., they can act as anti-inflammatory agents.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments, the subject is a mammal, e.g., a primate, e.g., a human. The terms, “patient” and “subject” are used interchangeably herein. The terms, “patient” and “subject” are used interchangeably herein.

Preferably, the subject is a mammal. The mammal can be a human, non-human primate, mouse, rat, dog, cat, horse, or cow, but are not limited to these examples. Mammals other than humans can be advantageously used as subjects that represent animal models of autoimmune diseases and disorders, and cancers.

A subject can be one who suffers from an inflammatory disease or disorder or a cancer. A subject can be one who has been previously diagnosed with or identified as suffering from or having an inflammatory disease or disorder, or a cancer. A subject can be one who is suspected of having or is predisposed for an inflammatory disease or disorder, or a cancer. A subject can be one who has undergone or is undergoing an organ/tissue transplant. Additionally, the subject can have already undergone or is currently undergoing a treatment regime for inhibiting an immune response.

In some embodiments, the method comprises diagnosing a subject for an inflammatory disease or cancer before on set of administration.

As used herein, an anti-inflammation treatment aims to prevent or slow down (lessen) an undesired physiological change or disorder, such as the development or progression of the inflammation. Beneficial or desired clinical results include, but are not limited to, alleviation of symptoms, diminishment of extent of disease, stabilized (i.e., not worsening) state of disease, delay or slowing of inflammation disease progression, amelioration or palliation of the disease state, and remission (whether partial or total), whether detectable or undetectable. An anti-inflammation treatment can also mean prolonging survival as compared to expected survival if not receiving treatment. An anti-inflammation treatment can also completely suppress the inflammation response.

As used herein, the term “inflammation” refers to any cellular processes that lead to the activation of caspase-1, or caspase-5, the production of cytokines IL-1 and IL-8, and/or the related downstream cellular events resulting from the actions of the cytokines thus produced, for example, fever, fluid accumulation, swelling, abscess formation, and cell death. As used herein, the term “inflammation” refers to both acute responses (i.e., responses in which the inflammatory processes are active) and chronic responses (i.e., responses marked by slow progression and formation of new connective tissue). Acute and chronic inflammation may be distinguished by the cell types involved. Acute inflammation often involves polymorphonuclear neutrophils; whereas chronic inflammation is normally characterized by a lymphohistiocytic and/or granulomatous response.

As used herein, the term “inflammation” includes reactions of both the specific and non-specific defense systems. A specific defense system reaction is a specific immune system reaction response to an antigen (possibly including an autoantigen). A non-specific defense system reaction is an inflammatory response mediated by leukocytes incapable of immunological memory. Such cells include granulocytes, macrophages, neutrophils and eosinophils. Examples of specific types of inflammation are diffuse inflammation, focal inflammation, croupous inflammation, interstitial inflammation, obliterative inflammation, parenchymatous inflammation, reactive inflammation, specific inflammation, toxic inflammation and traumatic inflammation.

As used herein, the term “specific defense system” is intended to refer to that component of the immune system that reacts to the presence of specific antigens. Inflammation is said to result from a response of the specific defense system if the inflammation is caused by, mediated by, or associated with a reaction of the specific defense system. Examples of inflammation resulting from a response of the specific defense system include the response to antigens such as rubella virus, autoimmune diseases such as lupus erythematosus, rheumatoid arthritis, Reynaud's syndrome, multiple sclerosis etc., delayed type hypersensitivity response mediated by T-cells, etc. Chronic inflammatory diseases and the rejection of transplanted tissue and organs are further examples of inflammatory reactions of the specific defense system.

As used herein, a reaction of the “non-specific defense system” is intended to refer to a reaction mediated by leukocytes incapable of immunological memory. Such cells include granulocytes and macrophages. As used herein, inflammation is said to result from a response of the nonspecific defense system, if the inflammation is caused by, mediated by, or associated with a reaction of the non-specific defense system. Examples of inflammation which result, at least in part, from a reaction of the non-specific defense system include inflammation associated with conditions such as: adult respiratory distress syndrome (ARDS) or multiple organ injury syndromes secondary to septicemia or trauma; reperfusion injury of myocardial or other tissues; acute glomerulonephritis; reactive arthritis; dermatoses with acute inflammatory components; acute purulent meningitis or other central nervous system inflammatory disorders; thermal injury; hemodialysis; leukophoresis; ulcerative colitis; Crohn's disease; necrotizing enterocolitis; granulocyte transfusion associated syndromes; and cytokine-induced toxicity. The term immune-mediated refers to a process that is either autoimmune or inflammatory in nature.

Exemplary inflammatory diseases can be an autoimmune disease. As used herein, the term “autoimmune disease” refers to a disorder wherein the immune system of a subject mounts a humoral or cellular immune response to the subject's own tissue or has intrinsic abnormalities in its tissues preventing proper cell survival without inflammation. Examples of autoimmune diseases include, but are not limited to, acute disseminated encephalomyelitis, acute glomerulonephritis, Addison's disease, adult-onset idiopathic hypoparathyroidism (AOIH), allergic inflammation such as allergic asthma, alopecia totalis, amyotrophic lateral sclerosis, ankylosing spondylitis, antiphospholipid antibody syndrome, aplastic anemia, atopic dermiatitis, autoimmune Addison's Disease, autoimmune aplastic anemia, autoimmune hemolytic anemia, autoimmune hepatitis, autoimmune polyglandular syndrome type I, autoinflammatory PAPA syndrome, Behcet's disease, Celiac disease, chronic active hepatitis, chronic fatigue syndrome CFS), contact hypersensitivity, CREST syndrome, Crohn's disease, Crohn's disease and ulcerative colitis, dermatomyositis, diabetes, dilated cardiomyopathy, eosinophilia-myalgia syndrome, epidermolisis bullosa acquisita (EBA), familial cold autoinflammatory syndrome, Familial Mediaterranean Fever, fibromyalgia (FM), giant cell arteritis, Goodpasture's syndrome, graft-versus-host disease, Graves' disease (overactive thyroid), Guillain-Barre syndrome, Hashimoto's disease, Hashimoto's thyroiditis (underactive thyroid), hemochromatosis, Henoch-Schonlein purpura, idiopathic IgA nephropathy, idiopathic thrombocytopenic purpura, inflammatory bowel disease, insulin-dependent diabetes mellitus (IDDM), juvenile rheumatoid arthritis, Lambert-Eaton syndrome, linear IgA dermatosis, lupus erythematosis, Muckle-Wells syndrome, multiple sclerosis (MS), Myasthenia gravis, myocarditis, narcolepsy, necrotizing vasculitis, neonatal lupus syndrome (NLE), neonatal onset multisystem inflammatory disease, nephrotic syndrome, opsoclonus myoclonus syndrome, optic neuritis, Ord's thyroiditis, pelvic inflammatory disease, pemphigoid, pemphigus, peripheral neuropathy, pernicious anaemia, polyarthritis in dogs, polyendocrine failure, polymyalgia rheumatica, polymyositis, primary biliary sclerosis/cirrhosis, primary sclerosing cholangitis, psoriasis, rapidlyprogressive glomerulonephritis (RPGN), Raynaud's phenomenon, Reiter's syndrome, rheumatoid arthritis, scleroderma, sclerosing cholangitis, Sjogren's syndrome, stiff-man syndrome and thyroiditis., systemic lupus erythematosus, Takayasu's arteritis, temporal arteritis/giant cell arteritis, type 1 diabetes mellitus, Type 1 diabetes mellitus, ulcerative colitis, vaculitis, vitiligo, warm autoimmune hemolytic anemia, and Wegener's granulomatosis.

As used herein, the term “cancer or carcinoma” refers to the disease which is characterized by an uncontrolled proliferation of abnormal cells capable of invading adjacent tissues and spreading to distant organs. Accordingly, “cancer” refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within an animal, or may be a non-tumorigenic cancer cell, such as a leukemia cell. In some circumstances, cancer cells will be in the form of a tumor; such cells may exist locally within an animal, or circulate in the blood stream as independent cells, for example, leukemic cells. Examples of cancer include but are not limited to breast cancer, a melanoma, adrenal gland cancer, biliary tract cancer, bladder cancer, brain or central nervous system cancer, bronchus cancer, blastoma, carcinoma, a chondrosarcoma, cancer of the oral cavity or pharynx, cervical cancer, colon cancer, colorectal cancer, esophageal cancer, gastrointestinal cancer, glioblastoma, hepatic carcinoma, hepatoma, kidney cancer, leukemia, liver cancer, lung cancer, lymphoma, non-small cell lung cancer, osteosarcoma, ovarian cancer, pancreas cancer, peripheral nervous system cancer, prostate cancer, sarcoma, salivary gland cancer, small bowel or appendix cancer, small-cell lung cancer, squamous cell cancer, stomach cancer, testis cancer, thyroid cancer, urinary bladder cancer, uterine or endometrial cancer, vulval cancer, and the like.

Other exemplary cancers include, but are not limited to, ACTH-producing tumors, acute lymphocytic leukemia, acute nonlymphocytic leukemia, cancer of the adrenal cortex, bladder cancer, brain cancer, breast cancer, cervical cancer, chronic lymphocytic leukemia, chronic myelocytic leukemia, colorectal cancer, cutaneous T-cell lymphoma, endometrial cancer, esophageal cancer, Ewing's sarcoma, gallbladder cancer, hairy cell leukemia, head & neck cancer, Hodgkin's lymphoma, Kaposi's sarcoma, kidney cancer, liver cancer, lung cancer (small and/or non-small cell), malignant peritoneal effusion, malignant pleural effusion, melanoma, mesothelioma, multiple myeloma, neuroblastoma, non-Hodgkin's lymphoma, osteosarcoma, ovarian cancer, ovary (germ cell) cancer, prostate cancer, pancreatic cancer, penile cancer, retinoblastoma, skin cancer, soft-tissue sarcoma, squamous cell carcinomas, stomach cancer, testicular cancer, thyroid cancer, trophoblastic neoplasms, uterine cancer, vaginal cancer, cancer of the vulva, Wilm's tumor, and the like

The term “transplant” as used herein refers to any organ or body tissue that has been transferred from its site of origin to a recipient site. Specifically in an allograft transplant procedure, the site of origin of the transplant is in a donor individual and the recipient site is in another, recipient individual. The term “organ” as used herein refers to a structure of bodily tissue in mammal such as a human being wherein the tissue structure as a whole is specialized to perform a particular body function. Organs that are transplanted within the meaning of the present methods include skin, cornea, heart, lung, kidney, liver and pancreas. Solid organs include the heart, lung, kidney, liver and pancreas.

For administration to a subject, the compounds can be provided in pharmaceutically acceptable compositions. These pharmaceutically acceptable compositions comprise a therapeutically-effective amount of one or more of the NFAT:transcription factor interaction inhibitors described herein, formulated together with one or more pharmaceutically acceptable carriers (additives) and/or diluents. As described in detail below, the pharmaceutical compositions of the present invention can be specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, compounds can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alchols, such as ethanol; and (23) other nontoxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

The amount of the compound that can be combined with a carrier material to produce a single dosage form will generally be that amount of the compound that produces a therapeutic effect. Generally out of one hundred percent, this amount will range from about 0.1% to 99% of compound, preferably from about 5% to about 70%, most preferably from 10% to about 30%.

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment. For example, an amount of a compound administered to a subject that is sufficient to produce a statistically significant, measurable change in the level of at least one pro-inflammatory mediator or a growth factor in the subject. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

The therapeutically effective dose can be estimated initially from cell culture assays. A dose may be formulated in animal models to achieve a circulating plasma concentration range that includes the IC50 (i.e., the concentration of the therapeutic which achieves a half-maximal inhibition of symptoms) as determined in cell culture. Levels in plasma may be measured, for example, by high performance liquid chromatography. The effects of any particular dosage can be monitored by a suitable bioassay.

Toxicity and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit large therapeutic indices, are preferred.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized.

The dosage can be determined by a physician and adjusted, as necessary, to suit observed effects of the treatment. Generally, the compositions are administered so that inflammasome inhibitor is given at a dose from 1 μg/kg to 150 mg/kg, 1 μg/kg to 100 mg/kg, 1 μg/kg to 50 mg/kg, 1 μg/kg to 20 mg/kg, 1 μg/kg to 10 mg/kg, 1 μg/kg to 1 mg/kg, 100 μg/kg to 100 mg/kg, 100 μg/kg to 50 mg/kg, 100 μg/kg to 20 mg/kg, 100 μg/kg to 10 mg/kg, 100 μg/kg to 1 mg/kg, 1 mg/kg to 100 mg/kg, 1 mg/kg to 50 mg/kg, 1 mg/kg to 20 mg/kg, 1 mg/kg to 10 mg/kg, 10 mg/kg to 100 mg/kg, 10 mg/kg to 50 mg/kg, or 10 mg/kg to 20 mg/kg. It is to be understood that ranges given here include all intermediate ranges, for example, the range 1 tmg/kg to 10 mg/kg includes 1 mg/kg to 2 mg/kg, 1 mg/kg to 3 mg/kg, 1 mg/kg to 4 mg/kg, 1 mg/kg to 5 mg/kg, 1 mg/kg to 6 mg/kg, 1 mg/kg to 7 mg/kg, 1 mg/kg to 8 mg/kg, 1 mg/kg to 9 mg/kg, 2 mg/kg to 10 mg/kg, 3 mg/kg to 10 mg/kg, 4 mg/kg to 10 mg/kg, 5 mg/kg to 10 mg/kg, 6 mg/kg to 10 mg/kg, 7 mg/kg to 10 mg/kg, 8 mg/kg to 10 mg/kg, 9 mg/kg to 10 mg/kg, and the like. It is to be further undertood that the ranges intermediate to the given above are also within the scope of this invention, for example, in the range 1 mg/kg to 10 mg/kg, dose ranges such as 2 mg/kg to 8 mg/kg, 3 mg/kg to 7 mg/kg, 4 mg/kg to 6 mg/kg, and the like.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to treatment regimen. The dosing schedule can vary from once a week to daily depending on a number of clinical factors, such as the subject's sensitivity to the polypeptides. The desired dose can be administered at one time or divided into subdoses, e.g., 2-4 subdoses and administered over a period of time, e.g., at appropriate intervals through the day or other appropriate schedule. Such sub-doses can be administered as unit dosage forms. In some embodiments, administration is chronic, e.g., one or more doses daily over a period of weeks or months. Examples of dosing schedules are administration daily, twice daily, three times daily or four or more times daily over a period of 1 week, 2 weeks, 3 weeks, 4 weeks, 1 month, 2 months, 3 months, 4 months, 5 months, or 6 months or more.

As used herein, the term “administer” refers to the placement of a composition into a subject by a method or route which results in at least partial localization of the composition at a desired site such that desired effect is produced. A compound or composition described herein can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In preferred embodiments, the compositions are administered by intravenous infusion or injection.

By “treatment”, “prevention” or “amelioration” of a disease or disorder is meant delaying or preventing the onset of such a disease or disorder, reversing, alleviating, ameliorating, inhibiting, slowing down or stopping the progression, aggravation or deterioration the progression or severity of a condition associated with such a disease or disorder. In one embodiment, the symptoms of a disease or disorder are alleviated by at least 5%, at least 10%, at least 20%, at least 30%, at least 40%, or at least 50%.

A goal of immune response suppression is to bring pro-inflammatory mediator levels down to as close to normal as is safely possible. Accordingly, in one embodiment, level of at least one pro-inflammatory mediator in the subject undergoing treatment is reduced by at least 5%, at least 10%, at least 20%, at least 30%, at least 40%, or at least 50% relative to a reference level. A reference level can be the level of the pro-inflammatory mediator in the subject before onset of treatment regime.

Exemplary pro-inflammatory mediators include, but are not limited to, pro-inflammatory cytokines, leukocytes, leukotiens, prostaglandins and other mediators involved in the initiation and maintenance of inflammation. Pro-inflammatory cytokines and inflammation mediators include, but are not limited to, IL-1-alpha, IL-1-beta, IL-6, IL-8, IL-11, IL-12, IL-17, IL-18, TNF-alpha, leukocyte inhibitory factor (LIF), IFN-gamma, Oncostatin M (OSM), ciliary neurotrophic factor (CNTF), TGF-beta, granulocyte-macrophage colony stimulating factor (GM-CSF), and chemokines that chemoattract inflammatory cells. A number of assays for in vivo state of inflammation are known in the art which can be utilized for measuring pro-inflammatory mediator levels. See for example U.S. Pat. Nos. 5,108,899 and 5,550,139, contents of both of which are herein incorporated by reference.

Exemplary growth factors include, but are not limited to, adrenomedullin (AM), angiopoietin (Ang), autocrine motility factor, beta2-microglobulin (BDGF II); bone morphogenetic proteins (BMPs), brain-derived neurotrophic factor (BDNF), epidermal growth factor (EGF), erythropoietin (EPO); fibroblast growth factor (FGF); glial cell line-derived neurotrophic factor (GDNF); granulocyte colony-stimulating factor (G-CSF); granulocyte macrophage colony-stimulating factor (GM-CSF); growth differentiation factor-9 (GDF9); hepatocyte growth factor (HGF); hepatoma-derived growth factor (HDGF); insulin-like growth factor (IGF); interferon-gamma (IFN [symbol] □ IL-1 (cofactor for IL-3 and IL-6, activates T cells); IL-2 (T-cell growth factor, stimulates IL-1 synthesis, and activates B-cells and NK cells); IL-3 (stimulates production of all non-lymphoid cells); IL-4 (growth factor for activated B cells, resting T cells, and mast cells); IL-5 (induces differentiation of activated B cells and eosinophils); IL-6 (stimulates Ig synthesis and growth factor for plasma cells); IL-7 (growth factor for pre-B cells); migration-stimulating factor; myostatin (GDF-8); nerve growth factor (NGF) and other neurotrophins; platelet-derived growth factor (PDGF); thrombopoietin (TPO); transforming growth factor alpha (TGF-α); transforming growth factor beta (TGF-β) tumour_necrosis_factor-alpha (TNF-α); vascular endothelial growth factor (VEGF); placental growth factor (PlGF); and the like.

In some embodiment, a compound can be administrated to a subject in combination with a pharmaceutically active agent. Exemplary pharmaceutically active compound include, but are not limited to, those found in Harrison's Principles of Internal Medicine, 13th Edition, Eds. T. R. Harrison et al. McGraw-Hill N.Y., NY; Physicians Desk Reference, 50th Edition, 1997, Oradell N.J., Medical Economics Co.; Pharmacological Basis of Therapeutics, 8th Edition, Goodman and Gilman, 1990; United States Pharmacopeia, The National Formulary, USP XII NF XVII, 1990; current edition of Goodman and Oilman's The Pharmacological Basis of Therapeutics; and current edition of The Merck Index, the complete contents of all of which are herein incorporated in their entirety. In some embodiments, pharmaceutically active agent includes those agents known in the art for treatment of cancer, inflammation or inflammation associated disorders, or infections.

In some embodiments, the pharmaceutically active agent is an anti-inflammatory agent. Exemplary anti-inflammatory agents include, but are not limited to, non-steroidal anti-inflammatory drugs (NSAIDs—such as aspirin, ibuprofen, or naproxen, coricosteroids (such as presnisone), anti-malarial medication (such as hydrochloroquine), methotrexrate, sulfasalazine, leflunomide, anti-TNF medications, cyclophosphamise and mycophenolate.

In some embodiments, the pharmaceutically active agent is a immune response modulator. As used herein, the term “immune response modulator” refers to compound (e.g., a small-molecule, antibody, peptide, nucleic acid, or gene therapy reagent) that inhibits autoimmune response in a subject. Without wishing to be bound by theory, an immune response modulator inhibits the autoimmune response by inhibiting the activity, activation, or expression of inflammatory cytokines (e.g., IL-12, IL-23 or IL-27), or STAT-4. Exemplary immune response modulators include, but are not limited to, members of the group consisting of Lisofylline (LSF) and the LSF analogs and derivatives described in U.S. Pat. No. 6,774,130, contents of which are herein incorporated by reference in their entirety.

The compound described herein and the pharmaceutically active agent can be administrated to the subject in the same pharmaceutical composition or in different pharmaceutical compositions (at the same time or at different times). When administrated at different times, the compound and the pharmaceutically active agent can be administered within 5 minutes, 10 minutes, 20 minutes, 60 minutes, 2 hours, 3 hours, 4, hours, 8 hours, 12 hours, 24 hours of administration of the other. When the compound and the pharmaceutically active agent are administered in different pharmaceutical compositions, routes of administration can be different.

### SOME SELECTED DEFINITIONS

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Other than in the operating examples, or where otherwise indicated, all numbers expressing quantities of ingredients or reaction conditions used herein should be understood as modified in all instances by the term “about.” The term “about” when used in connection with percentages may mean±1%.

The singular terms “a,” “an,” and “the” include plural referents unless context clearly indicates otherwise. Similarly, the word “or” is intended to include “and” unless the context clearly indicates otherwise.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

The terms “decrease”, “reduced”, “reduction”, “decrease” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

As used herein, a “conservative variant” is an amino acid sequence in which a first amino acid is replaced by a second amino acid or amino acid analog having at least one similar biochemical property, which can be, for example, similar size, charge, hydrophobicity or hydrogen bonding capacity. For example, a first hydrophobic amino acid can be conservatively substituted with a second (non-identical) hydrophobic amino acid such as alanine, valine, leucine, or isoleucine, or an analog thereof. Similarly, a first basic amino acid can be conservatively substituted with a second (non-identical) basic amino acid such as arginine or lysine, or an analog thereof. In the same way, a first acidic amino acid can be conservatively substituted with a second (non-identical) acidic amino acid such as aspartic acid or glutamic acid, or an analog thereof or an aromatic amino acid such as phenylalanine can be conservatively substituted with a second aromatic amino acid or amino acid analog, for example tyrosine.

Certain compounds of the present invention, and definitions of specific functional groups are also described in more detail below. For purposes of this invention, the chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito: 1999, the entire contents of which are incorporated herein by reference. Furthermore, it will be appreciated by one of ordinary skill in the art that the synthetic methods, as described herein, utilize a variety of protecting groups. By the term “protecting group,” has used herein, it is meant that a particular functional moiety, e.g., C, O, S, or N, is temporarily blocked so that a reaction can be carried out selectively at another reactive site in a multifunctional compound. In certain embodiments, a protecting group reacts selectively in good yield to give a protected substrate that is stable to the projected reactions; the protecting group must be selectively removed in good yield by readily available, preferably nontoxic reagents that do not attack the other functional groups; the protecting group forms an easily separable derivative (more preferably without the generation of new stereogenic centers); and the protecting group has a minimum of additional functionality to avoid further sites of reaction. As detailed herein, oxygen, sulfur, nitrogen, and carbon protecting groups may be utilized. Exemplary protecting groups are detailed herein, however, it will be appreciated that the present invention is not intended to be limited to these protecting groups; rather, a variety of additional equivalent protecting groups can be readily identified using the above criteria and utilized in the method of the present invention. Additionally, a variety of protecting groups are described in Protective Groups in Organic Synthesis, Third Ed. Greene, T. W. and Wuts, P. G., Eds., John Wiley & Sons, New York: 1999, the entire contents of which are hereby incorporated by reference. Furthermore, a variety of carbon protecting groups are described in Myers, A.; Kung, D. W.; Zhong, B.; Movassaghi, M.; Kwon, S. J. Am. Chem. Soc. 1999, 121, 8401-8402, the entire contents of which are hereby incorporated by reference.

It will be appreciated that the compounds, as described herein, may be substituted with any number of substituents or functional moieties. In general, the term “substituted” whether proceeded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example, of HDAC-associated diseases (e.g., cancer). The term “stable”, as used herein, preferably refers to compounds which possess stability sufficient to allow manufacture and which maintain the integrity of the compound for a sufficient period of time to be detected and preferably for a sufficient period of time to be useful for the purposes described herein.

The term “acyl”, as used herein, refers to a carbonyl-containing functionality, e.g., —C(═O)R, wherein R is an aliphatic, alycyclic, heteroaliphatic, heterocyclic, aryl, heteroaryl, (aliphatic)aryl, (heteroaliphatic)aryl, heteroaliphatic(aryl), or heteroaliphatic(heteroaryl) moiety, whereby each of the aliphatic, heteroaliphatic, aryl, or heteroaryl moieties is substituted or unsubstituted, or is a substituted (e.g., hydrogen or aliphatic, heteroaliphatic, aryl, or heteroaryl moieties) oxygen or nitrogen containing functionality (e.g., forming a carboxylic acid, ester, or amide functionality).

The term “aliphatic”, as used herein, includes both saturated and unsaturated, straight chain (i.e., unbranched) or branched aliphatic hydrocarbons, which are optionally substituted with one or more functional groups. As will be appreciated by one of ordinary skill in the art, “aliphatic” is intended herein to include, but is not limited to, alkyl, alkenyl, and alkynyl moieties. Thus, as used herein, the term “alkyl” includes straight and branched alkyl groups. An analogous convention applies to other generic terms such as “alkenyl”, “alkynyl”, and the like.

Furthermore, as used herein, the terms “alkyl”, “alkenyl”, “alkynyl”, and the like encompass both substituted and unsubstituted groups. In certain embodiments, as used herein, “lower alkyl” is used to indicate those alkyl groups (substituted, unsubstituted, branched or unbranched) having 1-6 carbon atoms.

In certain embodiments, the alkyl, alkenyl, and alkynyl groups employed in the invention contain 1-20 aliphatic carbon atoms. In certain other embodiments, the alkyl, alkenyl, and alkynyl groups employed in the invention contain 1-10 aliphatic carbon atoms. In yet other embodiments, the alkyl, alkenyl, and alkynyl groups employed in the invention contain 1-8 aliphatic carbon atoms. In still other embodiments, the alkyl, alkenyl, and alkynyl groups employed in the invention contain 1-6 aliphatic carbon atoms. In yet other embodiments, the alkyl, alkenyl, and alkynyl groups employed in the invention contain 14 carbon atoms. Illustrative aliphatic groups thus include, but are not limited to, for example, methyl, ethyl, n-propyl, isopropyl, allyl, n-butyl, sec-butyl, isobutyl, tert-butyl, n-pentyl, sec-pentyl, isopentyl, tert-pentyl, n-hexyl, sec-hexyl, moieties, and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl, and the like.

The term “alicyclic”, as used herein, refers to compounds which combine the properties of aliphatic and cyclic compounds and include but are not limited to cyclic, or polycyclic aliphatic hydrocarbons and bridged cycloalkyl compounds, which are optionally substituted with one or more functional groups. As will be appreciated by one of ordinary skill in the art, “alicyclic” is intended herein to include, but is not limited to, cycloalkyl, cycloalkenyl, and cycloalkynyl moieties, which are optionally substituted with one or more functional groups. Illustrative alicyclic groups thus include, but are not limited to, for example, cyclopropyl, —CH2-cyclopropyl, cyclobutyl, —CH2-cyclopentyl, cyclopentyl, —CH2-cyclopentyl, cyclohexyl, —CH2-cyclohexyl, cyclohexenylethyl, cyclohexanylethyl, norborbyl moieties, and the like, which may bear one or more substituents.

The term “alkoxy” or “alkyloxyl” or “thioalkyl”, as used herein, refers to an alkyl group, as previously defined, attached to the parent molecular moiety through an oxygen atom or through a sulfur atom. In certain embodiments, the alkyl group contains 1-20 aliphatic carbon atoms. In certain other embodiments, the alkyl group contains 1-10 aliphatic carbon atoms. In yet other embodiments, the alkyl group contains 1-8 aliphatic carbon atoms. In still other embodiments, the alkyl group contains 1-6 aliphatic carbon atoms. In yet other embodiments, the alkyl group contains 1-4 aliphatic carbon atoms. Examples of alkoxy, include, but are not limited to, methoxy, ethoxy, propoxy, isopropoxy, n-butoxy, tert-butoxy, neopentoxy, and n-hexoxy. Examples of thioalkyl include, but are not limited to, methylthio, ethylthio, propylthio, isopropylthio, n-butylthio, and the like.

The term “alkylamino” refers to a group having the structure —NHR′ wherein R′ is alkyl, as defined herein. The term “aminoalkyl” refers to a group having the structure NH2R′—, wherein R′ is alkyl, as defined herein. In certain embodiments, the alkyl group contains 1-20 aliphatic carbon atoms. In certain other embodiments, the alkyl group contains 1-10 aliphatic carbon atoms. In yet other embodiments, the alkyl contains 1-8 aliphatic carbon atoms. In still other embodiments, the alkyl group contains 1-6 aliphatic carbon atoms. In yet other embodiments, the alkyl group contains 1-4 aliphatic carbon atoms. Examples of alkylamino include, but are not limited to, methylamino, ethylamino, iso-propylamino, n-propylamino, and the like.

Some examples of substituents of the above-described aliphatic (and other) moieties of compounds of the invention include, but are not limited to, aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; —F; —Cl; —Br, —I; —OH; —NO2; —CN; —CF3; —CH2CF3; —CHCl2; —CH₂OH; —CH2CH2OH; —CH2NH2; —CH2SO2CH3; —C(O)Rx; —CO2(Rx); —CON(Rx)2; —OC(O)Rx; —OCO2Rx; —OCON(Rx)2; —N(Rx)2; —S(O)2Rx; —NRx(CO)Rx; wherein each occurrence of Rx independently includes, but is not limited to, aliphatic, alycyclic, heteroaliphatic, heterocyclic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substituents are illustrated by the specific embodiments described herein.

In general, the term “aromatic moiety”, as used herein, refers to a stable mono- or polycyclic, unsaturated moiety having preferably 3-14 carbon atoms, each of which may be substituted or unsubstituted. In certain embodiments, the term “aromatic moiety” refers to a planar ring having p-orbitals perpendicular to the plane of the ring at each ring atom and satisfying the Huckel rule where the number of pi electrons in the ring is (4n+2), wherein n is an integer. A mono- or polycyclic, unsaturated moiety that does not satisfy one or all of these criteria for aromaticity is defined herein as “non-aromatic,” and is encompassed by the term “alicyclic.”

In general, the term “heteroaromatic moiety” or, as used herein, refers to a stable mono- or polycyclic, unsaturated moiety having preferably 3-14 carbon atoms, each of which may be substituted or unsubstituted; and comprising at least one heteroatom selected from O, S, and N within the ring (i.e., in place of a ring carbon atom). In certain embodiments, the term “heteroaromatic moiety” refers to a planar ring comprising at least on heteroatom, having p-orbitals perpendicular to the plane of the ring at each ring atom, and satisfying the Huckel rule where the number of pi electrons in the ring is (4n+2), wherein n is an integer.

It will also be appreciated that aromatic and heteroaromatic moieties, as defined herein may be attached via an alkyl or heteroalkyl moiety and thus also include -(alkyl)aromatic, -(heteroalkyl)aromatic, -(heteroalkyl)heteroaromatic, and -(heteroalkyl)heteroaromatic moieties. Thus, as used herein, the phrases “aromatic or heteroaromatic moieties” and “aromatic, heteroaromatic, -(alkyl)aromatic, -(heteroalkyl)aromatic, -(heteroalkyl)heteroaromatic, and -(heteroalkyl)heteroaromatic” are interchangeable. Substituents include, but are not limited to, any of the previously mentioned substituents, i.e., the substituents recited for aliphatic moieties, or for other moieties as disclosed herein, resulting in the formation of a stable compound.

The term “aryl”, as used herein, does not differ significantly from the common meaning of the term in the art, and refers to an unsaturated cyclic moiety comprising at least one aromatic ring. In certain embodiments, “aryl” refers to a mono- or bicyclic carbocyclic ring system having one or two aromatic rings including, but not limited to, phenyl, naphthyl, tetrahydronaphthyl, indanyl, indenyl, and the like.

The term “heteroaryl”, as used herein, does not differ significantly from the common meaning of the term in the art, and refers to a cyclic aromatic radical having from five to ten ring atoms of which one ring atom is selected from S, O, and N; zero, one, or two ring atoms are additional heteroatoms independently selected from S, O, and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

It will be appreciated that aryl and heteroaryl groups (including bicyclic aryl groups) can be unsubstituted or substituted, wherein substitution includes replacement of one or more of the hydrogen atoms thereon independently with any one or more of the following moieties including, but not limited to aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; —F; —Cl; —Br, —I; —OH; —NO2; —CN; —CF3; —CH2CF3; —CHCl2; —CH2OH; —CH2CH2OH; —CH2NH2; —CH2SO2CH3; —C(O)Rx; —CO2(Rx); —CON(Rx)2; —OC(O)Rx; —OCO2Rx; —OCON(Rx)2; —N(Rx)2; —S(O)2Rx; and —NRx(CO)Rx; wherein each occurrence of Rx independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl, heteroaryl, -(alkyl)aryl or (alkyl)heteroaryl substituents described above and herein may be substituted or unsubstituted. Additionally, it will be appreciated, that any two adjacent groups taken together may represent a 4, 5, 6, or 7-membered substituted or unsubstituted alicyclic or heterocyclic moiety. Additional examples of generally applicable substituents are illustrated by the specific embodiments described herein.

The term “cycloalkyl”, as used herein, refers specifically to groups having three to seven, preferably three to ten carbon atoms. Suitable cycloalkyls include, but are not limited to cyclopropyl, cyclobutyl, cyclopentyl, cyclohexyl, cycloheptyl, and the like, which, as in the case of aliphatic, alicyclic, heteroaliphatic or heterocyclic moieties, may optionally be substituted with substituents including, but not limited to aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; —F; —Cl; —Br, —I; —OH; —NO2; —CN; —CF3; —CH2CF3; —CHCl2; —CH2OH; —CH2CH2OH; —CH2NH2; —CH2SO2CH3; —C(O)Rx; —CO2(Rx); —CON(Rx)2; —OC(O)Rx; —OCO2Rx; —OCON(Rx)2; —N(Rx)2; —S(O)2Rx; —NRx(CO)Rx; wherein each occurrence of Rx independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl or heteroaryl substituents described above and herein may be substituted or unsubstituted. Additional examples of generally applicable substituents are illustrated by the specific embodiments described herein.

The term “heteroaliphatic”, as used herein, refers to aliphatic moieties in which one or more carbon atoms in the main chain have been substituted with a heteroatom. Thus, a heteroaliphatic group refers to an aliphatic chain which contains one or more oxygen, sulfur, nitrogen, phosphorus or silicon atoms, e.g., in place of carbon atoms. Heteroaliphatic moieties may be linear or branched, and saturated or unsaturated. In certain embodiments, heteroaliphatic moieties are substituted by independent replacement of one or more of the hydrogen atoms thereon with one or more moieties including, but not limited to, aliphatic; alicyclic; heteroaliphatic; heterocyclic; aromatic; heteroaromatic; aryl; heteroaryl; alkylaryl; alkylheteroaryl; alkoxy; aryloxy; heteroalkoxy; heteroaryloxy; alkylthio; arylthio; heteroalkylthio; heteroarylthio; —F; —Cl; —Br, —I; —OH; —NO2; —CN; —CF3; —CH2CF3; —CHCl2; —CH2OH; —CH2CH2OH; —CH2NH2; —CH2SO2CH3; —C(O)Rx; —CO2(Rx); —CON(Rx)2; —OC(O)Rx; —OCO2Rx; —OCON(Rx)2; —N(Rx)2; —S(O)2Rx; and —NRx(CO)Rx; wherein each occurrence of Rx independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl or heteroaryl substituents described herein may be substituted or unsubstituted. Additional examples of generally applicable substituents are illustrated by the specific embodiments described herein.

The term “heterocycloalkyl”, “heterocycle” or “heterocyclic”, as used herein, refers to compounds which combine the properties of heteroaliphatic and cyclic compounds and include, but are not limited to, saturated and unsaturated mono- or polycyclic cyclic ring systems having 5-16 atoms wherein at least one ring atom is a heteroatom selected from O, S, and N (wherein the nitrogen and sulfur heteroatoms may be optionally be oxidized), wherein the ring systems are optionally substituted with one or more functional groups, as defined herein. In certain embodiments, the term “heterocycloalkyl”, “heterocycle” or “heterocyclic” refers to a non-aromatic 5-, 6-, or 7-membered ring or a polycyclic group wherein at least one ring atom is a heteroatom selected from O, S, and N (wherein the nitrogen and sulfur heteroatoms may be optionally be oxidized), including, but not limited to, a bi- or tri-cyclic group, comprising fused six-membered rings having between one and three heteroatoms independently selected from oxygen, sulfur and nitrogen, wherein (i) each 5-membered ring has 0 to 2 double bonds, each 6-membered ring has 0 to 2 double bonds and each 7-membered ring has 0 to 3 double bonds, (ii) the nitrogen and sulfur heteroatoms may be optionally be oxidized, (iii) the nitrogen heteroatom may optionally be quaternized, and (iv) any of the above heterocyclic rings may be fused to an aryl or heteroaryl ring. Representative heterocycles include, but are not limited to, heterocycles such as furanyl, thiofuranyl, pyranyl, pyrrolyl, thienyl, pyrrolidinyl, pyrazolinyl, pyrazolidinyl, imidazolinyl, imidazolidinyl, piperidinyl, piperazinyl, oxazolyl, oxazolidinyl, isooxazolyl, isoxazolidinyl, dioxazolyl, thiadiazolyl, oxadiazolyl, tetrazolyl, triazolyl, thiatriazolyl, oxatriazolyl, thiadiazolyl, oxadiazolyl, morpholinyl, thiazolyl, thiazolidinyl, isothiazolyl, isothiazolidinyl, dithiazolyl, dithiazolidinyl, tetrahydrofuryl, and benzofused derivatives thereof. In certain embodiments, a “substituted heterocycle, or heterocycloalkyl or heterocyclic” group is utilized and as used herein, refers to a heterocycle, or heterocycloalkyl or heterocyclic group, as defined above, substituted by the independent replacement of one, two or three of the hydrogen atoms thereon with but are not limited to aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroaryloxy; heteroaryloxy; alkylthio; arylthio; heteroalkylthio; heteroarylthio; F; Cl; Br, I; —OH; NO2; —CN; —CF3; —CH2CF3; —CHCl2; —CH2OH; —CH2CH2OH; —CH2NH2; —CH2SO2CH3; —C(O)Rx; —CO2 (Rx); —CON(Rx)2; —OC(O)Rx; —OCO2Rx; —OCON(Rx)2; —N(Rx)2; —S(O)2Rx; —NRx(CO)Rx; wherein each occurrence of Rx independently includes, but is not limited to, aliphatic, alicyclic; heteroaliphatic, heterocyclic, aromatic, heteroaromatic, aryl, heteroaryl, alkylaryl, alkylheteroaryl, heteroalkylaryl, or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl, or heteroaryl substituents described herein may be substituted or unsubstituted. Additional examples or generally applicable substituents are illustrated by the specific embodiments described herein.

Additionally, it will be appreciated that any of the alicyclic or heterocyclic moieties described herein may comprise an aryl or heteroaryl moiety fused thereto. Additional examples of generally applicable substituents are illustrated by the specific embodiments described herein.

The terms “halo” and “halogen” as used herein refer to an atom selected from fluorine, chlorine, bromine, and iodine.

The term “haloalkyl” denotes an alkyl group, as defined above, having one, two, or three halogen atoms attached thereto and is exemplified by such groups as chloromethyl, bromoethyl, trifluoromethyl, and the like. In certain embodiments, the alkyl group is perhalogenated (e.g., perfluorinated).

The term “amino”, as used herein, refers to a primary (—NH2), secondary (—NHRx), tertiary (—NRxRy), or quaternary (—N+RxRyRz) amine, where Rx, Ry, and Rz, are independently an aliphatic, alicyclic, heteroaliphatic, heterocyclic, aromatic or heteroaromatic moiety, as defined herein. Examples of amino groups include, but are not limited to, methylamino, dimethylamino, ethylamino, diethylamino, diethylaminocarbonyl, methylethylamino, iso-propylamino, piperidino, trimethylamino, and propylamino.

The term “carbamate”, as used herein, refers to any carbamate derivative known to one of ordinary skill in the art. Examples of carbamates include t-Boc, Fmoc, benzyloxy-carbonyl, alloc, methyl carbamate, ethyl carbamate, 9-(2-sulfo)fluorenylmethyl carbamate, 9-(2,7-dibromo)fluorenylmethyl carbamate, Tbfmoc, Climoc, Bimoc, DBD-Tmoc, Bsmoc, Troc, Teoc, 2-phenylethyl carbamate, Adpoc, 2-chloroethyl carbamate, 1,1-dimethyl-2-haloethyl carbamate, DB-t-BOC, TCBOC, Bpoc, t-Bumeoc, Pyoc, Bnpeoc, N-(2-pivaloylamino)-1,1-dimethylethyl carbamate, NpSSPeoc. In certain embodiments, carbamates are used as nitrogen protecting groups.

Unless otherwise indicated, as used herein, the terms “alkyl”, “alkenyl”, “alkynyl”, “heteroalkyl”, “heteroalkenyl”, “heteroalkynyl”, “alkylidene”, “alkynylidene”, -(alkyl)aryl, -(heteroalkyl)aryl, -(heteroalkyl)aryl, -(heteroalkyl)heteroaryl, and the like encompass substituted and unsubstituted, and linear and branched groups. Similarly, the terms “aliphatic”, “heteroaliphatic”, and the like encompass substituted and unsubstituted, saturated and unsaturated, and linear and branched groups. Similarly, the terms “cycloalkyl”, “heterocycle”, “heterocyclic”, and the like encompass substituted and unsubstituted, and saturated and unsaturated groups. Additionally, the terms “cycloalkenyl”, “cycloalkynyl”, “heterocycloalkenyl”, “heterocycloalkynyl”, “aromatic”, “heteroaromatic, “aryl”, “heteroaryl”, and the like encompass both substituted and unsubstituted groups.

The phrase, “pharmaceutically acceptable derivative”, as used herein, denotes any pharmaceutically acceptable salt, ester, or salt of such ester, of such compound, or any other adduct or derivative which, upon administration to a patient, is capable of providing (directly or indirectly) a compound as otherwise described herein, or a metabolite or residue thereof. Pharmaceutically acceptable derivatives thus include among others pro-drugs. A pro-drug is a derivative of a compound, usually with significantly reduced pharmacological activity, which contains an additional moiety, which is susceptible to removal in vivo yielding the parent molecule as the pharmacologically active species. An example of a pro-drug is an ester, which is cleaved in vivo to yield a compound of interest. Pro-drugs of a variety of compounds, and materials and methods for derivatizing the parent compounds to create the pro-drugs, are known and may be adapted to the present invention. The biological activity of pro-drugs may also be altered by appending a functionality onto the compound, which may be catalyzed by an enzyme. Also, included are oxidation and reduction reactions, including enzyme-catalyzed oxidation and reduction reactions. Certain exemplary pharmaceutical compositions and pharmaceutically acceptable derivatives are discussed in more detail herein.

The term “compound” or “chemical compound” as used herein can include organometallic compounds, organic compounds, metals, transitional metal complexes, and small molecules. In certain embodiments, polynucleotides are excluded from the definition of compounds. In other embodiments, polynucleotides and peptides are excluded from the definition of compounds. In certain embodiments, the term compound refers to small molecules (e.g., preferably, non-peptidic and non-oligomeric) and excludes peptides, polynucleotides, transition metal complexes, metals, and organometallic compounds.

As used herein, the term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts of amines, carboxylic acids, and other types of compounds, are well known in the art. For example, Berge et al. describe pharmaceutically acceptable salts in detail in J. Pharmaceutical Sciences 1977, 6, 1-19, incorporated herein by reference. The salts can be prepared in situ during the final isolation and purification of a compound of the invention, or separately by reacting a free base or free acid function with a suitable reagent, as described generally below. For example, a free base can be reacted with a suitable acid. Furthermore, where the compound of the invention carries an acidic moiety, suitable pharmaceutically acceptable salts thereof may, include metal salts such as alkali metal salts, e.g. sodium or potassium salts; and alkaline earth metal salts, e.g. calcium or magnesium salts. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid; or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

In some embodiments, the invention can be described by any one of the numbered paragraph:
1. An assay for identifying a test compound that inhibits NFAT:AP-1:DNA interactions comprising the steps of:
   forming a complex comprising a NFAT peptide, a Fos peptide, a Jun peptide, and an oligonucleotide, wherein the NFAT peptide comprises a DNA-binding domain (DBD), the Fos peptide and the Jun peptide each comprise a basic leucine zipper domain (bZIP domain), and the oligonucleotide comprises a NFAT binding site and a transcription factor binding site;
   measuring the amount, formation or stability of the complex in the presence of a test compound relative to a reference sample; and
   selecting the test compound that decreases the amount, formation or stability of the complex, wherein a decrease in amount, formation or stability of the complex in the presence of the test compound relative to the reference sample is indicative of a decrease in NFAT:AP-1:DNA interactions.
2. The assay of paragraph 1, wherein amount, formation or stability of the complex in the presence of the test compound decreases by at least 5% relative to the reference sample.
3. The assay of any of paragraphs 1-2, wherein the reference sample is chosen from one or more of a control sample not exposed to the test compound; a control sample exposed to an inhibitor of NFAT:AP-1 interaction; a control sample exposed to an inhibitor of the complex; or a control sample exposed to an excess amount of a complex member component.
4. The assay of any of paragraphs 1-3, wherein the evaluating step is a FRET-based assay.
5. The assay of paragraph 4, wherein the FRET-based assay is chosen from one or more of FRET or Time Resolved FRET assays (TR-FRET).
6. The assay of any of paragraphs 1-5, wherein at least one component of the complex comprises a FRET donor and at least one other component of the complex comprises a FRET acceptor.
7. The assay of paragraph 6, wherein the FRET donor and the FRET acceptor are selected from the group consisting of 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein (pH 10); 5-Carboxytetramethylrhodamine (5-TAMRA); 5-FAM (5-Carboxyfluorescein); 5-Hydroxy Tryptamine (HAT); 5-ROX (carboxy-X-rhodamine); 5-TAMRA (5-Carboxytetramethylrhodamine); 6-Carboxyrhodamine 6G; 6-CR 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Aequorin (Photoprotein); Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; AMCA (Aminomethylcoumarin); AMCA-X; Aminoactinomycin D; Aminocoumarin; Anilin Blue; Anthrocyl stearate; APC-Cy7; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Bimane; Bisbenzamide; Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; Calcein; Calcein Blue; Calcium Crimson™; Calcium Green; Calcium Green-1 Ca2+ Dye; Calcium Green-2 Ca2+; Calcium Green-5N Ca2+; Calcium Green-C18 Ca2+; Calcium Orange; Calcofluor White; Carboxy-X-rhodamine (5-ROX); Cascade Blue™; Cascade Yellow; Catecholamine; CFDA; CFP—Cyan Fluorescent Protein; Chlorophyll; Chromomycin A; Chromomycin A; CMFDA; Coelenterazine; Coelenterazine cp; Coelenterazine f; Coelenterazine fcp; Coelenterazine h; Coelenterazine hcp; Coelenterazine ip; Coelenterazine O; Coumarin Phalloidin; CPM Methylcoumarin; CTC; Cy2™; Cy3.1 8; Cy3.5™; Cy3™; Cy5.1 8; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA; DCFH (Dichlorodihydrofluorescein Diacetate); DDAO; DHR (Dihydrorhodamine 123); Di-4-ANEPPS; Di-8-ANEPPS (non-ratio); DiA (4-Di-16-ASP); DIDS; Dihydrorhodamine 123 (DHR); DiO (DiOC18(3)); DiR; DiR (DiIC18(7)); Dopamine; DsRed; DTAF; DY-630—NHS; DY-635—NHS; EBFP; ECFP; EGFP; ELF 97; Eosin; Erythrosin; Erythrosin ITC; Ethidium homodimer-1 (EthD-1); Euchrysin; Europium (III) chloride; Europium; EYFP; Fast Blue; FDA; Feulgen (Pararosaniline); FITC; FL-645; Flazo Orange; Fluo-3; Fluo-4; Fluorescein Diacetate; Fluoro-Emerald; Fluoro-Gold (Hydroxystilbamidine); Fluor-Ruby; Fluor X; FM 1-43™; FM 4-46; Fura Red™ (high pH); Fura-2, high calcium; Fura-2, low calcium; Genacryl Brilliant Red B; Genacryl Brilliant Yellow 10GF; Genacryl Pink 3G; Genacryl Yellow 5GF; GFP(S65T); GFP red shifted (rsGFP); GFP wild type, non-UV excitation (wtGFP); GFP wild type, UV excitation (wtGFP); GFPuv; Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indodicarbocyanine (DiD); Indotricarbocyanine (DiR); Intrawhite Cf; JC-1; JO-JO-1; JO-PRO-1; LaserPro; Laurodan; LDS 751; Leucophor PAF; Leucophor SF; Leucophor WS; Lissamine Rhodamine; Lissamine Rhodamine B; LOLO-1; LO-PRO-1; Lucifer Yellow; Mag Green; Magdala Red (Phloxin B); Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxadidole; Noradrenaline; Nuclear Fast Red; Nuclear Yellow; Nylosan Brilliant Iavin EBG; Oregon Green™; Oregon Green 488-X; Oregon Green™ 488; Oregon Green™ 500; Oregon Green™ 514; Pacific Blue; Pararosaniline (Feulgen); PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26; PKH67; PMIA; Pontochrome Blue Black; POPO-1; POPO-3; PO-PRO-1; PO-PRO-3; Primuline; Procion Yellow; Propidium Iodid (PI); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B 540; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycoerythrin (PE); red shifted GFP (rsGFP, S65T); S65A; S65C; S65L; S65T; Sapphire GFP; Serotonin; Sevron Brilliant Red 2B; Sevron Brilliant Red 4G; Sevron Brilliant Red B; Sevron Orange; Sevron Yellow L; sgBFP™; sgBFP™ (super glow BFP); sgGFP™; sgGFP™ (super glow GFP); SITS; SITS (Primuline); SITS (Stilbene Isothiosulphonic Acid); SPQ (6-methoxy-N-(3-sulfopropyl)-quinolinium); Stilbene; Sulphorhodamine B can C; Sulphorhodamine G Extra; Tetracycline; Tetramethylrhodamine; Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC (TetramethylRodamineIsoThioCyanate); True Blue; TruRed; Ultralite; Uranine B; Uvitex SFC; wt GFP; WW 781; XL665; X-Rhodamine; XRITC; Xylene Orange; Y66F; Y66H; Y66W; Yellow GFP; YFP; YO-PRO-1; YO-PRO-3; YOYO-1; YOYO-3; and any combinations thereof.
8. The assay of any of paragraphs 6-7, wherein the FRET donor is Oregon Green 488 (OG-488).
9. The assay of any of paragraphs 6-8, wherein the FRET acceptor is Alexa 546.
10. The assay of any of paragraphs 6-9, wherein one of Fos peptide or the Jun peptide comprises the FRET donor.
11. The assay of any of paragraphs 6-10, wherein Fos peptide comprises the FRET donor.
12. The assay of any of paragraphs 6-11, wherein oligonucleotide comprises the FRET acceptor.
13. The assay of any of paragraphs 6-12, wherein FRET donor fluorescence changes by at least 5% in the presence of the test compound relative to the reference sample.
14. The assay of any of paragraphs 6-13, wherein FRET acceptor fluorescence changes by at least 5% in the presence of the test compound relative to the reference sample
15. The assay of any of paragraphs 1-14, wherein the transcription site is an AP-1 binding site.
16. The assay of any of paragraphs 1-15, wherein the oligonucleotide comprises the nucleotide sequence 5′-GGAAAATTTGTTTCATAG-3′ (SEQ ID NO: 3) or 5′-CTG TAT GAA ACA AAT TTT CCT CTT TG-3′ (SEQ ID NO: 4).
17. The assay of any of paragraphs 1-16, wherein the oligonucleotide comprises at least one modification selected from the group consisting of sugar modifications, phosphorothioate intersugar linkages, non-phosphodiester intersugar linkages, nucleobase modifications, and any combinations thereof.
18. The assay of any of paragraphs 1-17, wherein the NFAT peptide comprises the amino acid sequence:

(SEQ ID NO: 6)
MRGSHHHHHHTDPHASSVPLEWPLSSQSGSYELRIEVQPKPHHRAHYET
EGSRGAVKAPTGGHPVVQLHGYMENKPLGLQIFIGTADERILKPHAFYQ
VHRITGKTVTTTSYEKIVGNTKVLEIPLEPKNNMRATIDCAGILKLRNA
DIELRKGETDIGRKNTRVRLVFRVHIPESSGRIVSLQTASNPIECSQRS
AHELPMVERQDTDSCLVYGGQQMILTGQNFTSESKVVFTEKTTDGQQIW
EMEATVDKDKSQPNMLFVEIPEYRNKHIRTPVKVNFYVINGKRKRSQPQ
HFTYHP.

19. The assay of any of paragraphs 1-18, wherein the Fos peptide comprises the amino acid sequence:

(SEQ ID NO: 1)
MKRRIRRERNKMAAAKSRNRRRELTDTLQAETDQLEDEKSALQTEIANLL
KEKEKLEFILAAH.

20. The assay of any of paragraphs 1-19, wherein the Jun peptide comprises the amino acid sequence:

(SEQ ID NO: 2)
MKAERKRMRNRIAASKSRKRKLERIARLEEKVKTLKAQNSELASTANMLR
EQVAQLKQKVMNH.

21. The assay of any of paragraphs 1-20, wherein at least one of NFAT peptide, Fos peptide, or Jun peptide comprises a modification selected from the group consisting of D-amino acids, a beta-amino-acids, a gamma-amino acids, chemically modified amino acids, modified amide linkages, or any combinations thereof.
22. The assay of any of paragraphs 1-21, wherein the test compound is selected from the group consisting of small or larger organic or inorganic molecules; saccharides; oligosaccharides; polysaccharides; peptides; proteins; peptide analogs and derivatives; peptidomimetics; nucleic acids; nucleic acid analogs and derivatives; extracts made from biological materials; animal cells; animal tissues; naturally occurring or synthetic compositions; and any combinations thereof.
23. The method of paragraph any of claims 1-22, wherein the test compound has a molecular weight of less than 5000 Daltons (5 kD).
24. The method of any of paragraphs 1-23, wherein the test compound is tested at a concentration in the range of about 0.1 nM to about 1000 mM.
25. The assay of any of paragraphs 1-24, wherein the sample further comprises a competitor of at least one component of the complex.
26. The assay of paragraph 25, wherein the competitor is a Fos-peptide, a Jun-peptide, a NFAT peptide, an oligonucleotide comprising the NFAT binding site, or an oligonucleotide comprising the transcription binding site.
27. The assay of any of paragraphs 25-26, wherein the competitor is an oligonucleotide comprising a NFAT binding site or a transcription binding site.
28. The assay of any of paragraphs 25-27, wherein the competitor is present at a concentration in the range of about 0.1 nM to about 1000 mM.
29. The assay of any of paragraphs 1-28, wherein the assay is a high-throughput screening assay.
30. A compound selected by the assay of any of paragraphs 1-29 and derivatives, isomers and pharmaceutically acceptable salts thereof.
31. A pharmaceutical composition comprising a compound of paragraph 30 and a pharmaceutically acceptable carrier.
32. A method of inhibiting NFAT:AP-1:DNA interactions in a cell, the method comprising contacting a cell with a compound of paragraph 30.
33. The method of paragraph 32, wherein the compound is of formula (I):

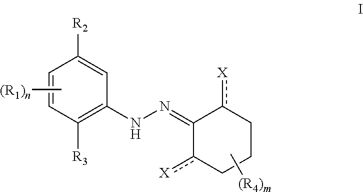

wherein
   each R₁, R₂, R₃, and R₄ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; OR^{B}; —C(═O)R^{B}; —CO₂R^{B}; —C(═O)N(R^{B})₂; —CN; —SCN; —SR^{B}; —SOR^{B}; —SO₂R^{B}; —NO₂; —N(R^{B})₂; —NHC(O)R^{B}; or —C(R^{B})₃; wherein each occurrence of R^{B} is independently hydrogen; halogen; a protecting group; aliphatic; heteroaliphatic; acyl; aryl moiety; heteroaryl; hydroxyl; aloxy; aryloxy; alkylthioxy; arylthioxy; amino; alkylamino; dialkylamino; heteroaryloxy; heteroarylthioxy; alkylhalo;
   each X is independently H, O, or R₄,
   n is an integer 0-3, inclusive;
   m is an integer 0-5, inclusive;
   or salt thereof.
34. The method of paragraph 33, wherein each R₁, R₂, R₃, and R₄ is independently hydrogen; halogen; substituted or unsubstituted C₁₋₆ alkyl, OR^{B}, —C(═O)R^{B}, —CO₂R^{B}, —C(═O)N(R^{B})₂, —CN, —SCN, —SR^{B}, —SOR^{B}, —SO₂R^{B}, —NO₂, —N(R^{B})₂, —NHC(O)R^{B}, or —C(R^{B})₃, wherein each occurrence of R^{B} is independently hydrogen, halogen, a protecting group, substituted or unsubstituted C₁₋₆ alkyl, hydroxyl, alkoxy, amino, alkylamino, alkylhalo, or salt thereof.
35. The method of paragraph 33, wherein each R₁, R₃, and R₄ is independently hydrogen; halogen; substituted or unsubstituted C₁₋₆ alkyl, OR^{B}, —C(═O)R^{B}, —CO₂R^{B}, —C(═O)N(R^{B})₂, —CN, —SCN, —SR^{B}, —SOR^{B}, —SO₂R^{B}, —NO₂, —N(R^{B})₂, —NHC(O)R^{B}, or —C(R^{B})₃, wherein each occurrence of R^{B} is independently hydrogen, halogen, a protecting group, substituted or unsubstituted C₁₋₆ alkyl, hydroxyl, alkoxy, amino, alkylamino, alkylhalo, or salt thereof.
36. The method of paragraph 33, wherein each R₁, R₃, and R₄ is independently hydrogen; halogen, substituted or unsubstituted Cₕ₆ alkyl, OR^{B}, —C(═O)R^{B}, —CO₂R^{B}, —CN, —SCN, —SR^{B}, —SOR^{B}, —SO₂R^{B}, —NO₂, —N(R^{B})₂, —NHC(O)R^{B}, or —C(R^{B})₃, wherein each occurrence of R^{B} is independently hydrogen, halogen, a protecting group, C₁₋₆ alkyl, or salt thereof; R₂ is C₁₋₆ alkyl, halogen, or CF₃, R₃ is OR^{B}, wherein R^{B} is hydrogen, halogen, a protecting group, substituted or unsubstituted Cₕ₆ alkyl, alkoxy, amino, alkylamino, or alkylhalo; X is O, n is an integer 0-3, inclusive; and m is an integer 0-3, inclusive.
37. The method of paragraph 32, wherein the compound is of formula (II):

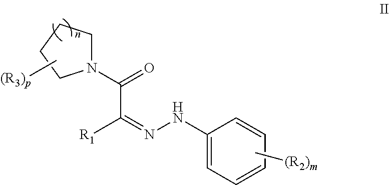

wherein
   each R₁, R₂, and R₃, is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; OR^{B}; —C(═O)R^{B}; —CO₂R^{B}; —C(═O)N(R^{B})₂; —CN; —SCN; —SR^{B}; —SOR^{B}; —SO₂R^{B}; —NO₂; —N(R^{B})₂; —NHC(O)R^{B}; or —C(R^{B})₃; wherein each occurrence of R^{B} is independently hydrogen; halogen; a protecting group; aliphatic; heteroaliphatic; acyl; aryl moiety; heteroaryl; hydroxyl; aloxy; aryloxy; alkylthioxy; arylthioxy; amino; alkylamino; dialkylamino; heteroaryloxy; heteroarylthioxy; alkylhalo; and two R₂ can be taken together to form a five or six membered ring;
   n is an integer 0-3, inclusive;
   m is an integer 0-5, inclusive;
   p is an integer 0-7, inclusive;
   or salt thereof.
38. The method of paragraph 37, wherein each R₁, R₂, and R₄ is independently hydrogen; halogen, substituted or unsubstituted C₁₋₆ alkyl, OR^{B}, —C(═O)R^{B}, —CO₂R^{B}, —C(═O)N(R^{B})₂, —CN, —SCN, —SR^{B}, —SOR^{B}, —SO₂R^{B}, —NO₂, —N(R^{B})₂, —NHC(O)R^{B}, or —C(R^{B})₃, wherein each occurrence of R^{B} is independently hydrogen, halogen, a protecting group, substituted or unsubstituted Cₕ₆ alkyl, hydroxyl, alkoxy, amino, alkylamino, or alkylhalo.
39. The method of paragraph 37, wherein R₁ is hydrogen, halogen, substituted or unsubstituted C₁₋₆ alkyl, OR^{B}, —C(═O)R^{B}, —CO₂R^{B}, —C(═O)N(R^{B})₂, —CN, —SCN, —SR^{B}, —SOR^{B}, —SO₂R^{B}, —NO₂, —N(R^{B})₂, —NHC(O)R^{B}, or —C(R^{B})₃, wherein each occurrence of R^{B} is independently hydrogen, halogen, a protecting group, substituted or unsubstituted C₁₋₆ alkyl, hydroxyl, alkoxy, amino, alkylamino, or alkylhalo; R₂ is an acetal; m is 2; n is an integer 0-1, inclusive; and p is 0.
40. The method of paragraph 37, wherein R₁ is —CN; R₂ is a five membered acetal; m is 2; n is an integer 0-1, inclusive; and p is 0.
41. The method of any of paragraphs 32-40, wherein the contact is in in vitro.
42. The method of any of paragraphs 32-40, wherein the contact is in in vivo.
43. The method of paragraph 42, wherein the in vivo contact is in a mammal.
44. The method of any of paragraphs 42-43, wherein the in vivo contact is in a non-human.
45. The method of any of paragraphs 42-44, wherein the in vivo contact is in a mouse or rat.
46. The method of any of paragraphs 42-43, wherein the in vivo contact is in a human.
47. The method of any of paragraphs 42-46, wherein the in vivo contact is in a subject, which subject suffers from a disease condition characterized by an elevated level immune response.
48. The method of any of paragraphs 42-47, wherein the in vivo contact is in a subject, which subject has elevated level of at least one pro-inflammatory mediator relative to a reference level.
49. The method of paragraph 48, wherein the pro-inflammatory mediator is selected from the group consisting of IL-1-alpha, IL-1-beta, IL-6, IL-8, IL-11, IL-12, IL-17, IL-18, TNF-alpha, leukocyte inhibitory factor (LIF), IFN-gamma, Oncostatin M (OSM), ciliary neurotrophic factor (CNTF), TGF-beta, granulocyte-macrophage colony stimulating factor (GM-CSF), chemokines that chemoattract inflammatory cells, and any combinations thereof.
50. The method of any of paragraphs 42-49, wherein the in vivo contact is in a subject, which subject suffers from an inflammatory disease or disorder, cancer, or complications after a tissue/organ transplant.
51. The method of paragraph 50, wherein the inflammatory disease or disorder is an autoimmune disease.
52. The method of any of paragraphs 32-49, wherein the compound is

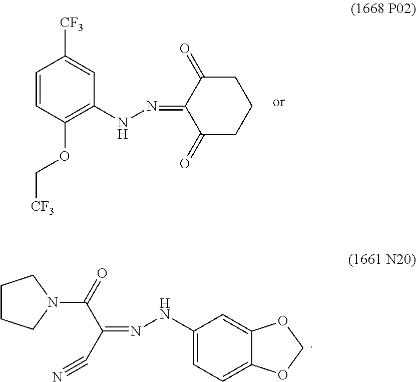 [SMILES: O=C1CCCC(=O)C1=NNc1cc(C(F)(F)F)ccc1OCC(F)(F)F ; N#C/C(=N/Nc1ccc2c(c1)OCO2)C(=O)N1CCCC1]

53. A method of inhibiting an immune response in a subject, the method comprising administering a therapeutically effective amount of a compound of paragraph 30 to a subject in need thereof.
54. The method of paragraph 53, wherein the compound is of formula (I):

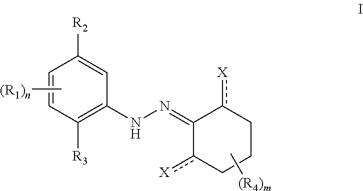

wherein
   each R₁, R₂, R₃, and R₄ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; OR^{B}; —C(═O)R^{B}; —CO₂R^{B}; —C(═O)N(R^{B})₂; —CN; —SCN; —SR^{B}; —SOR^{B}; —SO₂R^{B}; —NO₂; —N(R^{B})₂; —NHC(O)R^{B}; or —C(R^{B})₃; wherein each occurrence of R^{B} is independently hydrogen; halogen; a protecting group; aliphatic; heteroaliphatic; acyl; aryl moiety; heteroaryl; hydroxyl; aloxy; aryloxy; alkylthioxy; arylthioxy; amino; alkylamino; dialkylamino; heteroaryloxy; heteroarylthioxy; alkylhalo;
   each X is independently H, O, or R₄,
   n is an integer 0-3, inclusive;
   m is an integer 0-5, inclusive;
   or salt thereof.
55. The method of paragraph 54, wherein each R₁, R₂, R₃, and R₄ is independently hydrogen; halogen; substituted or unsubstituted C₁₋₆ alkyl, OR^{B}, —C(═O)R^{B}, —CO₂R^{B}, —C(═O)N(R^{B})₂, —CN, —SCN, —SR^{B}, —SOR^{B}, —SO₂R^{B}, —NO₂, —N(R^{B})₂, —NHC(O)R^{B}, or —C(R^{B})₃, wherein each occurrence of R^{B} is independently hydrogen, halogen, a protecting group, substituted or unsubstituted Cₕ₆ alkyl, hydroxyl, alkoxy, amino, alkylamino, alkylhalo, or salt thereof.
56. The method of paragraph 54, wherein each R₁, R₃, and R₄ is independently hydrogen; halogen; substituted or unsubstituted C₁₋₆ alkyl, OR^{B}, —C(═O)R^{B}, —CO₂R^{B}, —C(═O)N(R^{B})₂, —CN, —SCN, —SR^{B}, —SOR^{B}, —SO₂R^{B}, —NO₂, —N(R^{B})₂, —NHC(O)R^{B}, or —C(R^{B})₃, wherein each occurrence of R^{B} is independently hydrogen, halogen, a protecting group, substituted or unsubstituted C₁₋₆ alkyl, hydroxyl, alkoxy, amino, alkylamino, alkylhalo, or salt thereof.
57. The method of paragraph 54, wherein each R₁, R₃, and R₄ is independently hydrogen; halogen, substituted or unsubstituted C₁₋₆ alkyl, OR^{B}, —C(═O)R^{B}, —CO₂R^{B}, —CN, —SCN, —SR^{B}, —SOR^{B}, —SO₂R^{B}, —NO₂, —N(R^{B})₂, —NHC(O)R^{B}, or —C(R^{B})₃, wherein each occurrence of R^{B} is independently hydrogen, halogen, a protecting group, C₁₋₆ alkyl, or salt thereof; R₂ is C₁₋₆ alkyl, halogen, or CF₃, R₃ is OR^{B}, wherein R^{B} is hydrogen, halogen, a protecting group, substituted or unsubstituted Cₕ₆ alkyl, alkoxy, amino, alkylamino, or alkylhalo; X is O, n is an integer 0-3, inclusive; and m is an integer 0-3, inclusive.
58. The method of paragraph 53, wherein the compound is of formula (II):

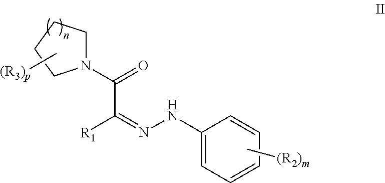

wherein
   each R₁, R₂, and R₃, is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; OR^{B}; —C(═O)R^{B}; —CO₂R^{B}; —C(═O)N(R^{B})₂; —CN; —SCN; —SR^{B}; —SOR^{B}; —SO₂R^{B}; —NO₂; —N(R^{B})₂; —NHC(O)R^{B}; or —C(R^{B})₃; wherein each occurrence of R^{B} is independently hydrogen; halogen; a protecting group; aliphatic; heteroaliphatic; acyl; aryl moiety; heteroaryl; hydroxyl; aloxy; aryloxy; alkylthioxy; arylthioxy; amino; alkylamino; dialkylamino; heteroaryloxy; heteroarylthioxy; alkylhalo; and two R₂ can be taken together to form a five or six membered ring;
   n is an integer 0-3, inclusive;
   m is an integer 0-5, inclusive;
   p is an integer 0-7, inclusive;
   or salt thereof.
59. The method of paragraph 58, wherein each R₁, R₂, and R₄ is independently hydrogen; halogen, substituted or unsubstituted C₁₋₆ alkyl, OR^{B}, —C(═O)R^{B}, —CO₂R^{B}, —C(═O)N(R^{B})₂, —CN, —SCN, —SR^{B}, —SOR^{B}, —SO₂R^{B}, —NO₂, —N(R^{B})₂, —NHC(O)R^{B}, or —C(R^{B})₃, wherein each occurrence of R^{B} is independently hydrogen, halogen, a protecting group, substituted or unsubstituted C₁₋₆ alkyl, hydroxyl, alkoxy, amino, alkylamino, or alkylhalo.
60. The method of paragraph 58, wherein R₁ is hydrogen, halogen, substituted or unsubstituted C₁₋₆ alkyl, OR^{B}, —C(═O)R^{B}, —CO₂R^{B}, —C(═O)N(R^{B})₂, —CN, —SCN, —SR^{B}, —SOR^{B}, —SO₂R^{B}, —NO₂, —N(R^{B})₂, —NHC(O)R^{B}, or —C(R^{B})₃, wherein each occurrence of R^{B} is independently hydrogen, halogen, a protecting group, substituted or unsubstituted C₁₋₆ alkyl, hydroxyl, alkoxy, amino, alkylamino, or alkylhalo; R₂ is an acetal; m is 2; n is an integer 0-1, inclusive; and p is 0.
61. The method of paragraph 58, wherein R₁ is —CN; R₂ is a five membered acetal; m is 2; n is an integer 0-1, inclusive; and p is 0.
62. The method of any of paragraphs 53-61, wherein the subject is a mammal.
63. The method of any of paragraphs 53-62, wherein the subject is a human.
64. The method of any of paragraphs 53-63, wherein the subject suffers from a disease condition characterized by an elevated level immune response.
65. The method of any of paragraphs 53-64, wherein the subject has elevated level of at least one pro-inflammatory mediator relative to a reference level.
66. The method of paragraph 65, wherein the pro-inflammatory mediator is selected from the group consisting of IL-1-alpha, IL-1-beta, IL-6, IL-8, IL-11, IL-12, IL-17, IL-18, TNF-alpha, leukocyte inhibitory factor (LIF), IFN-gamma, Oncostatin M (OSM), ciliary neurotrophic factor (CNTF), TGF-beta, granulocyte-macrophage colony stimulating factor (GM-CSF), chemokines that chemoattract inflammatory cells, and any combinations thereof.
67. The method of any of paragraphs 53-66, wherein the subject suffers from an inflammatory disease or disorder, cancer, or complications after a tissue/organ transplant.
68. The method of paragraph 67, wherein the inflammatory disease or disorder is an autoimmune disease.
69. The method of any of paragraphs 53-68, wherein the compound is

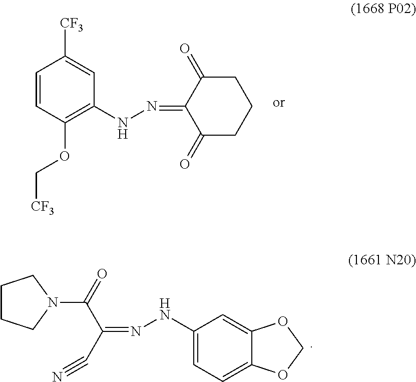 [SMILES: O=C1CCCC(=O)C1=NNc1cc(C(F)(F)F)ccc1OCC(F)(F)F ; N#C/C(=N/Nc1ccc2c(c1)OCO2)C(=O)N1CCCC1]

70. The method of any of paragraphs 53-69, wherein the therapeutically effective amount is 1 μg/kg to 150 mg/kg body weight of the subject.
71. The method of any of paragraphs 53-70, wherein a pharmaceutically active agent is also administered to the subject.
72. The method of paragraph 71, wherein the therapeutically active agent is an anti-inflammatory agent or an immune response modulator.
73. The method of paragraph 72, wherein the anti-inflammatory agent is selected from the group consisting of non-steroidal anti-inflammatory drugs (NSAIDs—such as aspirin, ibuprofen, or naproxen, coricosteroids (such as presnisone), anti-malarial medication (such as hydrochloroquine), methotrexrate, sulfasalazine, leflunomide, anti-TNF medications, cyclophosphamise, mycophenolate, and any combinations thereof.

To the extent not already indicated, it will be understood by those of ordinary skill in the art that any one of the various embodiments herein described and illustrated may be further modified to incorporate features shown in any of the other embodiments disclosed herein.

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art can develop equivalent means or reactants without the exercise of inventive capacity and without departing from the scope of the invention.

### Materials and Methods

Protein purification: Fos and Jun bZIP domains (each encompassing 63 amino acids) were cloned into pETa vectors and used to transform Codon Plus™ BL21 (DE3) RIL E. Coli cells; redox sensitive Cys 154 in Fos and Cys 272 in Jun had been previously mutated to Ser. Fos and Jun bZIP peptides were purified from the corresponding bacterial cultures by cation exchange chromatography using SP (Sulfopropyl) Sepharose following the manufacturer's instructions (GE Biosciences). A unique Cys residue proximal to the Fos N-terminal was introduced via site-directed mutagenesis in the Fos bZIP construct (Ile 142→Cys) and the Fos I→C mutant was purified in the same way using SP Sepharose. The NFAT DBD containing a His tag was cloned into the pQE vector and the protein was purified by nickel affinity chromatography (HiTrap Resin; GE Biosciences).

Fluorescent labeling: Oregon Green 488-maleimide (Invitrogen) was dissolved in DMSO and added to the Fos I→C peptide (in PBS) at a 5-fold molar excess. The labeling reaction was allowed to proceed for 2-3 h at room temperature and terminated by the addition of 1M DTT to quench free dye. OG 488-labeled Fos peptide was purified from the crude mixture by means of reverse phase (C8) HPLC.

FRET assay: Fos-OG 488, ARRE2-Alexa 546, Jun and the consensus AP-1 oligo were used at a final concentration of 10 or 20 nM in binding buffer (20 mM Tris pH=7.5, 50 mM NaCl, 5% glycerol, 0.5 mg/mL BSA) in low volume 384-well assay plates (Corning). NFAT was titrated in at up to a 5-fold molar excess. The mixture was incubated at room temperature for 25-30′ and the plate was read using a Synergy 2 instrument (Biotek) using 485/20 nm and 528/20 nm filters for absorption and emission respectively. Siliconized pipette tips were used to dispense protein/DNA solutions in order to minimize losses due to surface adhesion during transfer. The labeled ARRE2 oligo (5′-CCT TCT GTA TGA AAC AAA TTT TCC TCT TTG-3′) (SEQ ID NO: 7), AP-1 oligo (5′-TCT CCT ATG ACT CAT CC-3′) (SEQ ID NO: 5) and respective complement oligos were ordered from IDT.

For measuring dissociation kinetics, 20 nM Fos-OG/ARRE2-Alexa 546/Jun were incubated with 40 nM NFAT or 40 nM NFAT-RIT or buffer alone for 30′; donor fluorescence was measured (t=0). Either unlabeled Fos (200 nM) or unlabeled AP-1 oligo (20 nM) were added to the binding mixture and donor fluorescence was monitored at ˜1′ intervals after addition of competitor until equilibrium was attained.

High throughput screening: NFAT was added at 80 nM in a volume of 6 μL to each well of a 384-well plate, followed by pin-transfer of small molecule compounds (33 nL per well) from three commercially available chemical libraries (IF lab2, Maybridge 2 and Maybridge 5) at the Institute of Chemistry and Cellular Biology (ICCB; Longwood Medical Area). Plates were read after pin transfer to identify intrinsically fluorescent compounds in the libraries. Next, 6 μL of assay mixture containing 20 nM Fos-OG/ARRE2-Alexa 546/Jun/AP-1 oligo was added to each well. The plates were read following 30′ incubation at room temperature. The screen was performed in duplicate; each plate contained a set of control wells—DMSO transferred into the assay mix containing either NFAT, as in the experimental wells, or buffer alone.

Analysis of Results: Wells containing fluorescent compounds were first excluded from the analysis. Mean fluorescence value and standard deviation (SD) were calculated for each plate; wells with readings ≧3 SD+mean were designated as outliers. Plate statistics were recalculated without outlier wells and the corrected mean and SD were used to calculate z-scores for each well; z=(x−mean)/SD where x=donor fluorescence value for a given well. Wells with z-score≧2 for each replicate were considered strong hits while wells with z-score≧1.5 for one replicate and ≧2 for the other replicate were considered moderate.

EMSAs: Probe labeling: Annealed double-stranded ARRE2 was end-labeled using T4 polynucleotide kinase by incubating 50-100 ng DNA with 20 μCi 32P-γATP (Perkin Elmer) at 37° C. for 1 h followed by heat inactivation of the enzyme at 65° C. for 15′ and purification of radiolabeled ARRE2 using MicroSpin G-25 columns (GE Biosciences).

Binding reactions were set up in EMSA buffer (10 mM HEPES pH=7.5, 120 mM NaCl, 10% glycerol, 0.8 mg/mL BSA, 1 mM DTT) in the presence of complete protease inhibitor (Roche). Compounds CID 2740816 (1668 P02) and CID 5713461 (1661 N20) (Ryan Scientific) were resuspended in DMSO and incubated with 20 nM NFAT and 10 nM AP-1 for 10′ followed by addition of ˜2.5 nM radiolabeled ARRE2 probe. Reactions were then incubated for 30′ at room temperature and loaded on a 4% polyacrylamide gel (pre-run for 30-45′).

T cell activation assays: Mouse CD4 T cells were isolated using the Dynal CD4 T cell positive isolation kit (Invitrogen) and activated with 1 μg/mL αCD3/αCD28 for 48-60 h under non-polarizing or Th1-conditions (10 ng/mL IL-12 and 10 μg/mL αIL-4). CD4CD25+ T cells were depleted from purified CD4 T cells using a CD25 microbead MACS kit (Miltenyi Biotech), and CD4CD25− T cells were activated with αCD3/αCD28 in the presence of 3 ng/mL TGF□ (iTreg-polarizing conditions).

For restimulation, T cells from primary cultures were rested for 3-4 d, washed, counted and stimulated for 4 h with 10 nM PMA and varied concentrations of ionomycin; where indicated, CsA was added to cells at a final concentration of 2 μM 20′ before activation while compounds were added 45-60′ before activation. Brefeldin A was added at 10 μg/mL for the final 2-2.5 h of stimulation. T cells were fixed with 4% paraformaldehyde, permeabilized in saponin buffer and stained for intracellular cytokines; data were collected on a FACSCalibur instrument (BD Biosciences).

EAE induction: CD4 T cells from 2D2 transgenic mice [1] were activated with 0.2 μg/mL αCD3/αCD28 under Th1-polarizing conditions for 48-60 h in the presence of DMSO or compound as indicated. T cells were rested and expanded for 2 d followed by restimulation with 0.1 μg/mL αCD3/αCD28 for 24 h in the presence of DMSO or compound. Cells were resuspended in 1×PBS and injected intraperitoneally into female 8 wk old C57BL/6 mice, 1.5 million T cells per mouse. Mice were monitored for weight loss and development of disease symptoms using a standard clinical scale: 0, healthy; 1, flaccid tail; 2, ataxia/paresis of hind limbs; 3, paralysis of hind limbs; 4, tetraparalysis; 5, moribund or death W.

Real time RT-PCR: Total RNA was isolated from cell pellets using the RNeasy kit (Qiagen) followed by first-strand synthesis using SuperScript III (Invitrogen). Synthesized cDNA was amplified using the FastStart Universal SYBRGreen Master Mix (Roche). PCR data was collected using the StepOne Plus Real-Time PCR System (Applied Biosystems) and gene expression normalized to an endogenous GAPDH control using the ΔΔCT method; primer sequences have been described elsewhere [2].

### Results and Discussion

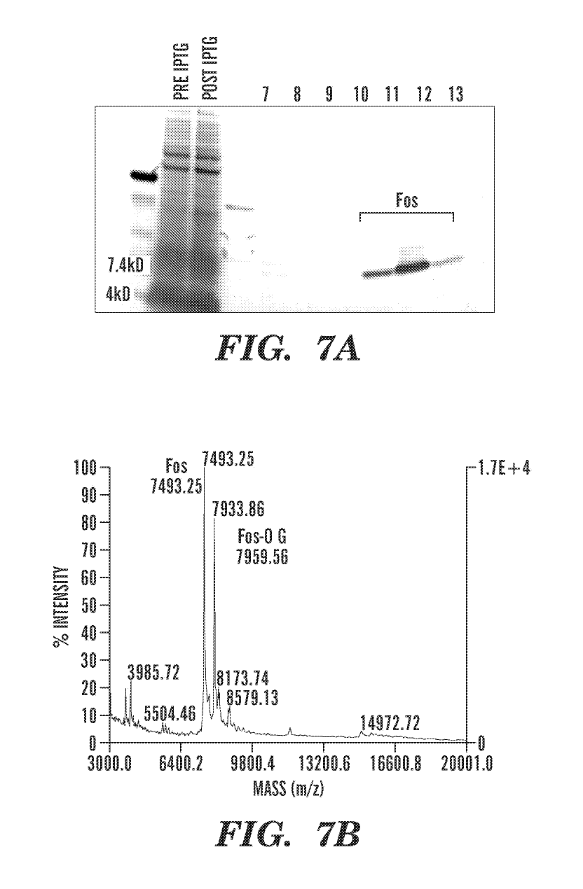

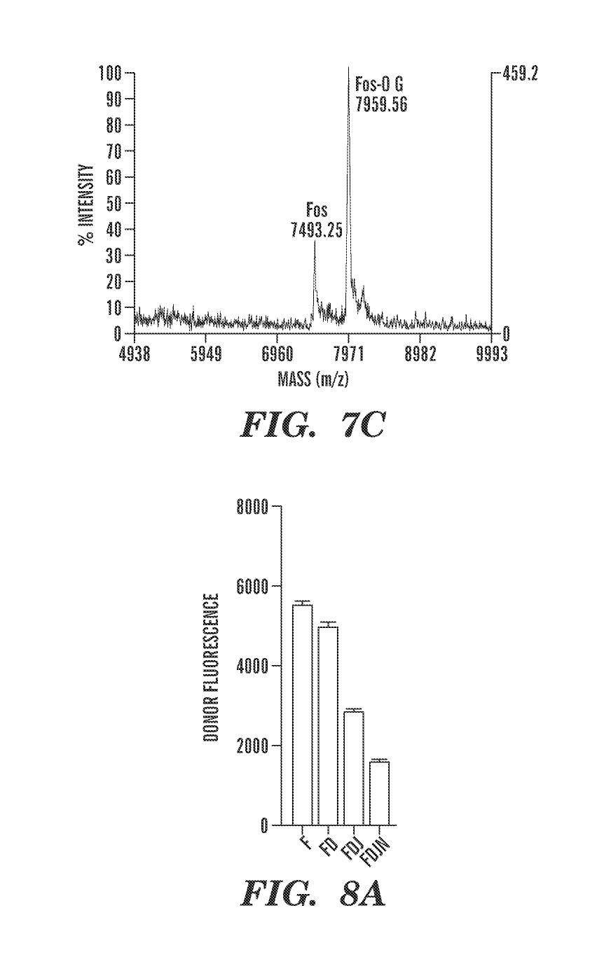

The NFAT DNA-binding domain (DBD) and basic region leucine zipper regions (bZIP) of Fos and Jun were bacterially expressed and purified using nickel affinity columns for NFAT-DBD and cation exchange chromatography for the AP-1 peptides (FIG. 7A). A unique Cys residue was introduced at position 142 of the Fos bZIP domain by means of a Ile→Cys mutation—the modified Fos peptide was labeled using Oregon Green (OG)-488 maleimide via a thioether linkage at Cys142, and purified by HPLC (FIG. 7B).

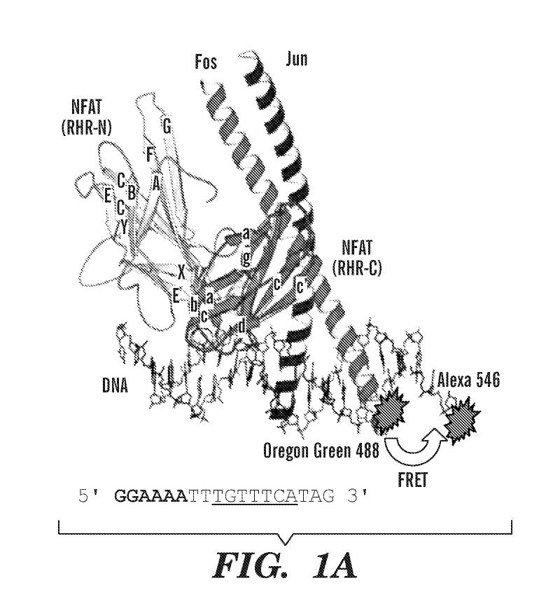

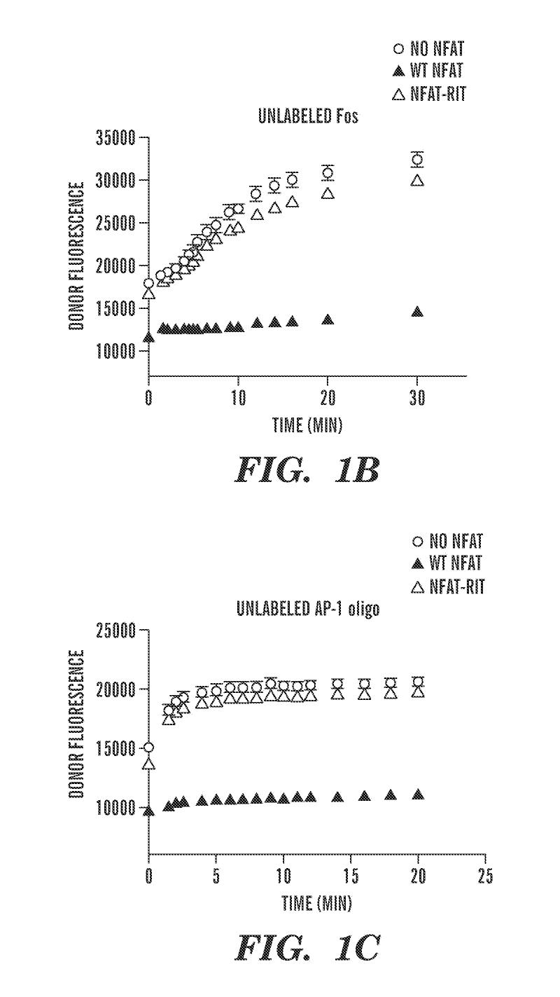

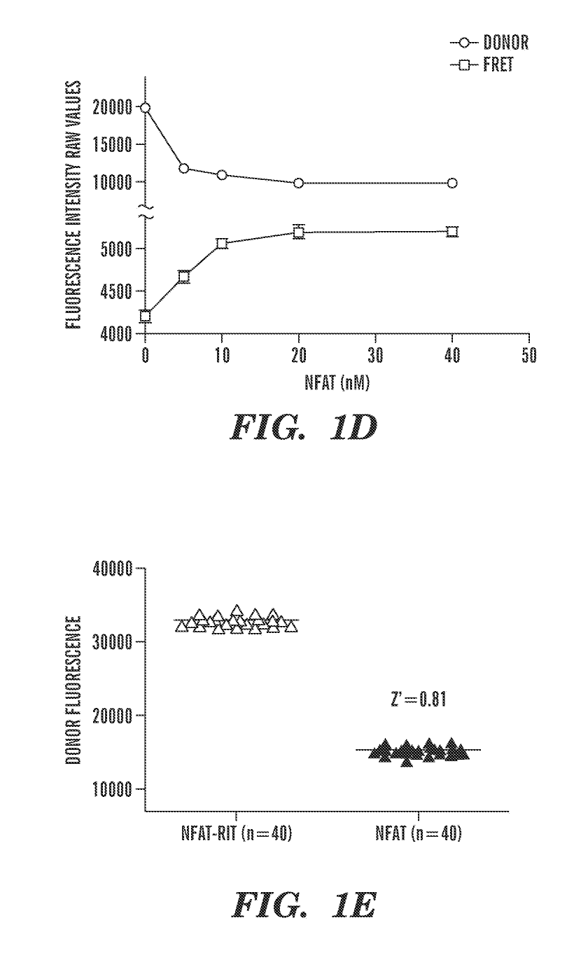

Development of a FRET assay to measure binding of NFAT to AP-1 and DNA: The inventors devised a FRET-based binding assay to measure assembly of the ternary NFAT:Fos:Jun:DNA complex, using OG-488 (on Fos) and Alexa 546 (on DNA) as the donor-acceptor pair. For DNA scaffold, inventors chose the antigen receptor response element (ARRE2) from the mouse IL-2 promoter. The mARRE2 sequence contains a classical NFAT:AP-1 composite element comprising a strong NFAT site (marked in bold face; FIG. 1A) and a non-consensus AP-1 site (underlined; FIG. 1A), with the precise dinucleotide intersite spacing which facilitates NFAT:AP-1 cooperation [3, 4].

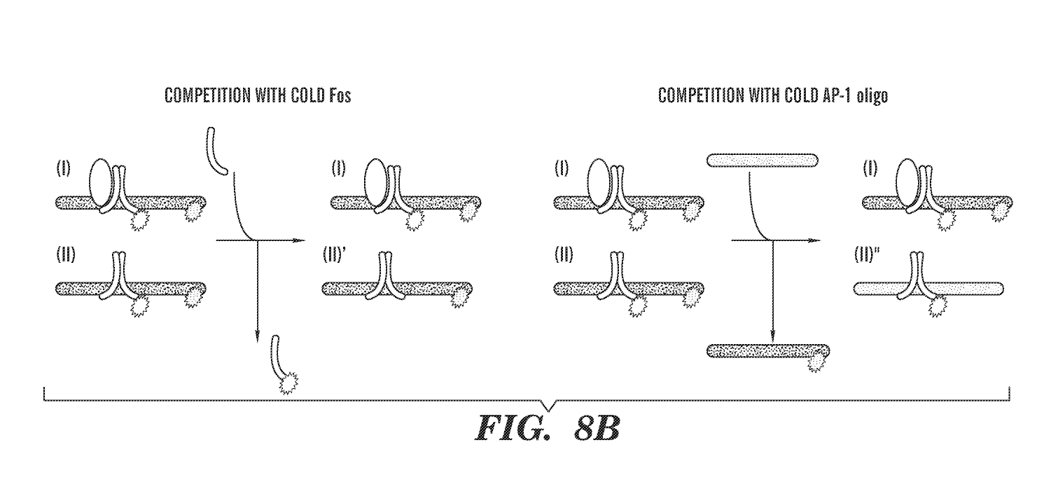

While NFAT addition led to a definite increase in the FRET signal, there appeared to be background binding of AP-1 to ARRE2 DNA even in the absence of NFAT (FIG. 8A)—this could be due to weak binding of AP-1 to its non-consensus ARRE2 site, non-specific electrostatic interactions between the Fos/Jun peptides and the DNA phosphodiester backbone or a combination of both [4, 5]. To circumvent this, and confirm assembly of a truly cooperative NFAT:AP-1: DNA complex, the inventors assessed the dissociation kinetics of either complex using an unlabeled Fos peptide or a competitor oligonucleotide containing a consensus AP-1 site (FIGS. 1B and 1C). Upon addition of either cold competitor, the background FRET signal observed in the absence of NFAT, ascribed to the non-specific AP-1: DNA complex, was rapidly lost pointing towards its facile dissociation (FIGS. 1B, 1C, 8B and 8C). As expected, the complex assembled in the presence of NFAT, comprising NFAT:AP-1: DNA, was substantially more stable, reduction in FRET occurring at a much slower rate (FIGS. 1B, 1C, 8B and 8C). For further confirmation, the inventors used a mutated version of NFAT, NFAT-RIT, containing mutations which abrogate its binding to AP-1-NFAT-RIT retains its DNA binding ability, the triad of R(→A), I(→A) and T(→G) mutations selectively disrupting its AP-1 binding interface [4, 6]. Predictably, the dissociation curves obtained with NFAT-RIT closely mirrored those obtained in the absence of NFAT binding (FIGS. 1B and 1C; compare the blue circles to yellow triangles).

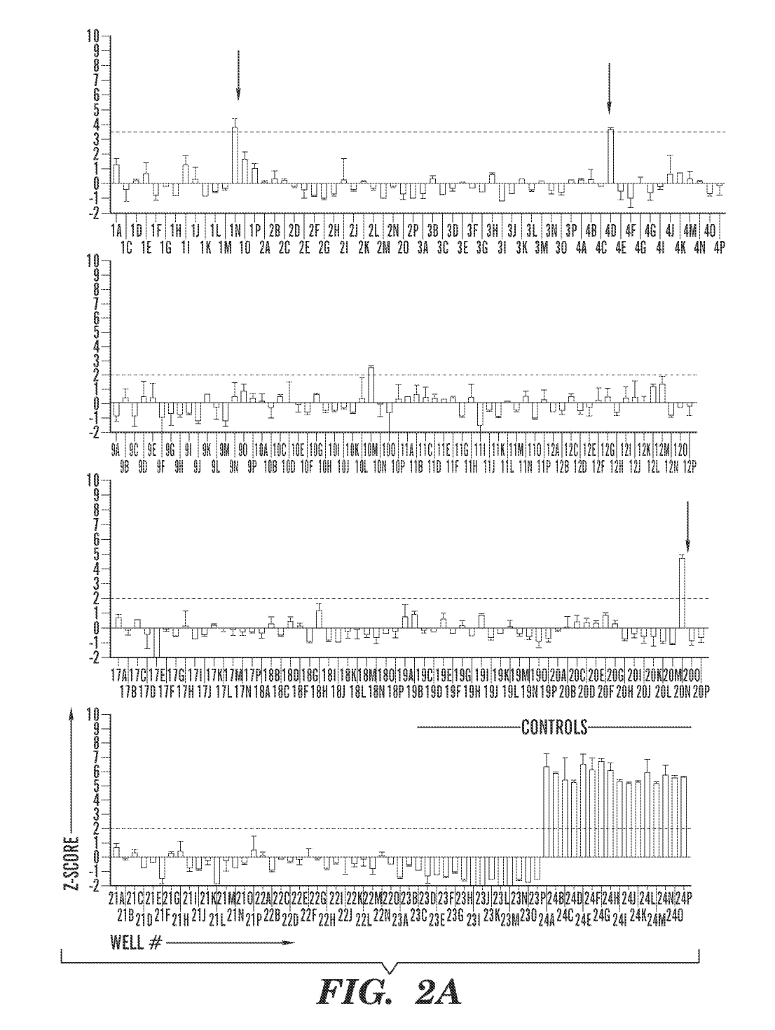

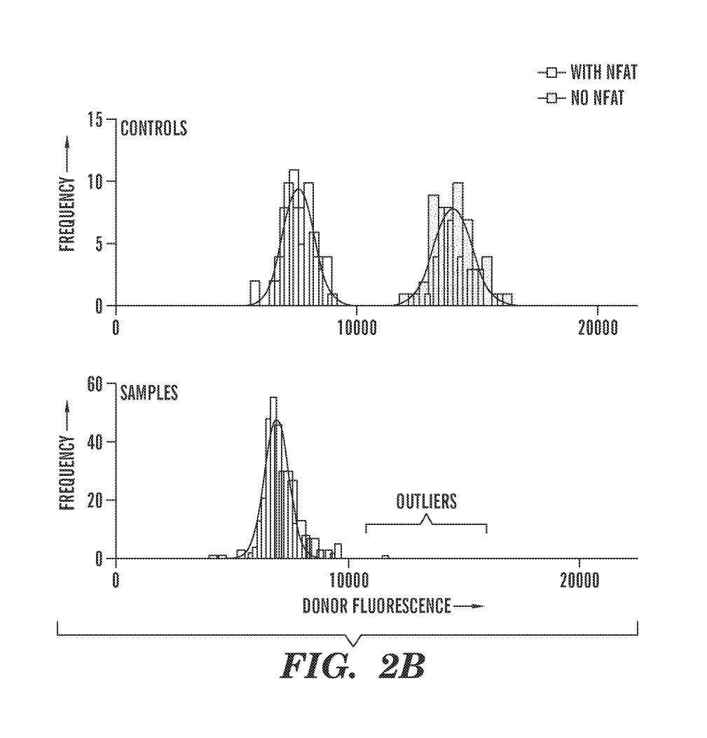

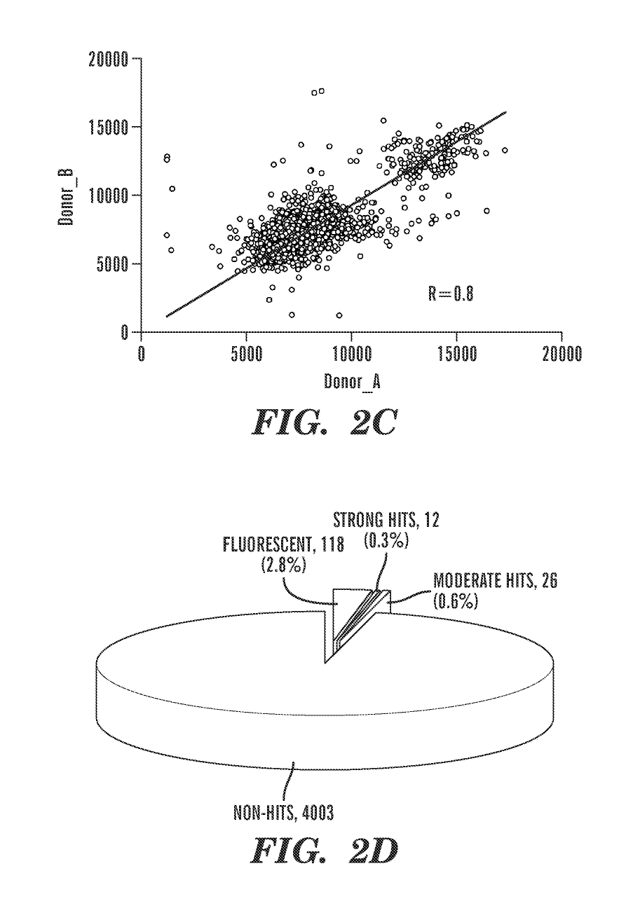

Performance of the screen and identification of hits: Loss of background AP-1/DNA binding led to an increase in the signal-to-noise ratio of the binding assay (FRET efficiency of ˜50% in the presence of an equimolar amount of the competitor oligo, relative to ˜30% in its absence)—the inventors obtained robust Z′ factors in the range of 0.75-0.8 (FIGS. 1D and 1E). The binding assay was adapted to a high throughput format to screen commercial chemical libraries at the Institute of Chemistry and Cellular Biology (See Materials and Methods). Dequenching of donor (Fos-OG) fluorescence was a read-out for inhibition of FRET, and hence assembly of the NFAT:AP-1: DNA complex (FIGS. 2A and 2B). Fluorescent compounds were identified by performing pin-transfers into wells containing NFAT alone in buffer; the fluorescent components of the assay, Fos-OG 488 and DNA-Alexa 546, were added subsequent to this (See Materials and Methods). The inventors tested a total of 4003 compounds in duplicate 384-well plates (correlation coefficient=0.8; FIG. 2C)—118 compounds (2.8%) were fluorescent and excluded from further analysis. The inventors identified 12 strong hits (z-score of 2 or more for each replicate) and 26 moderate hits (z-score between 1.5 and 2 for each replicate) (summarized in FIG. 2D).

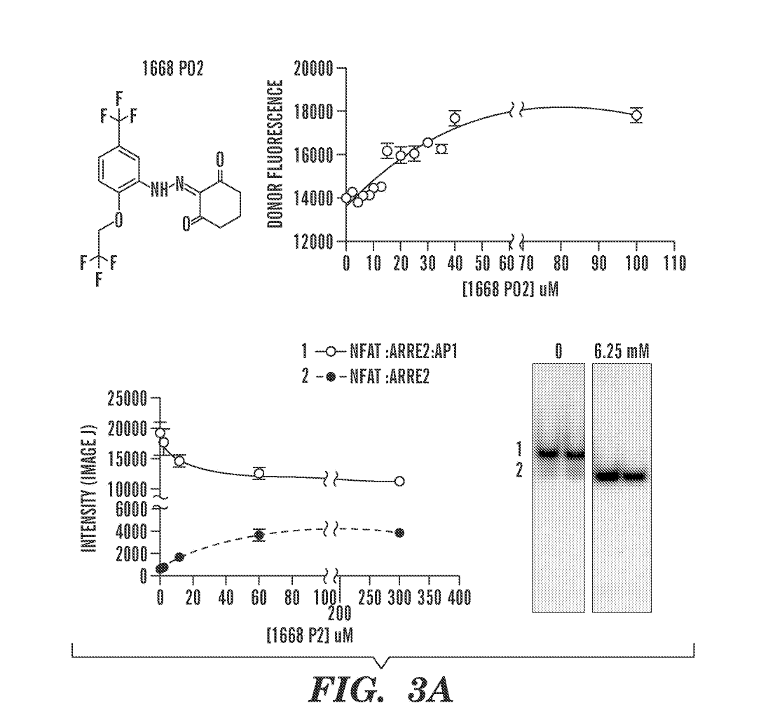

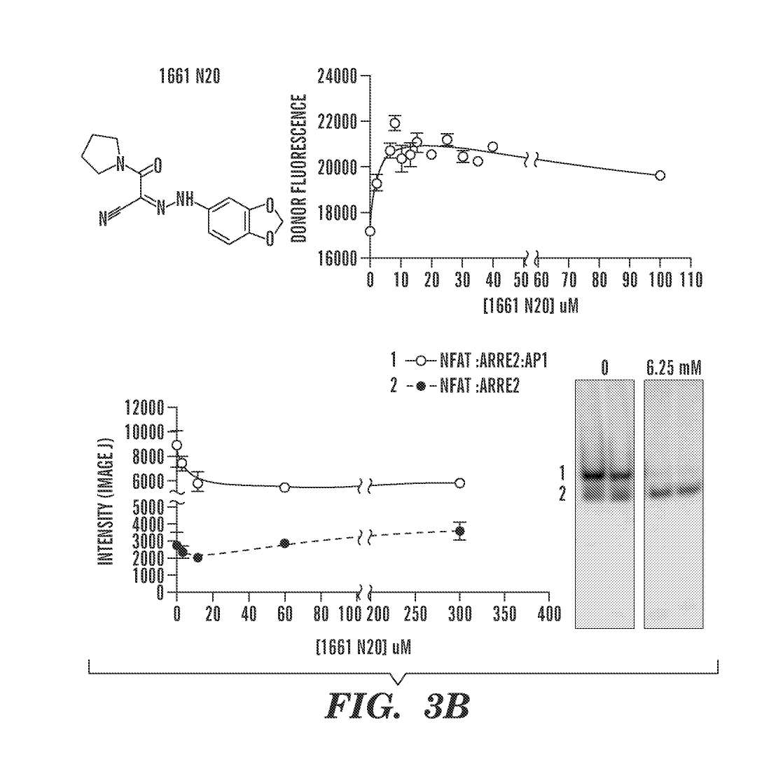

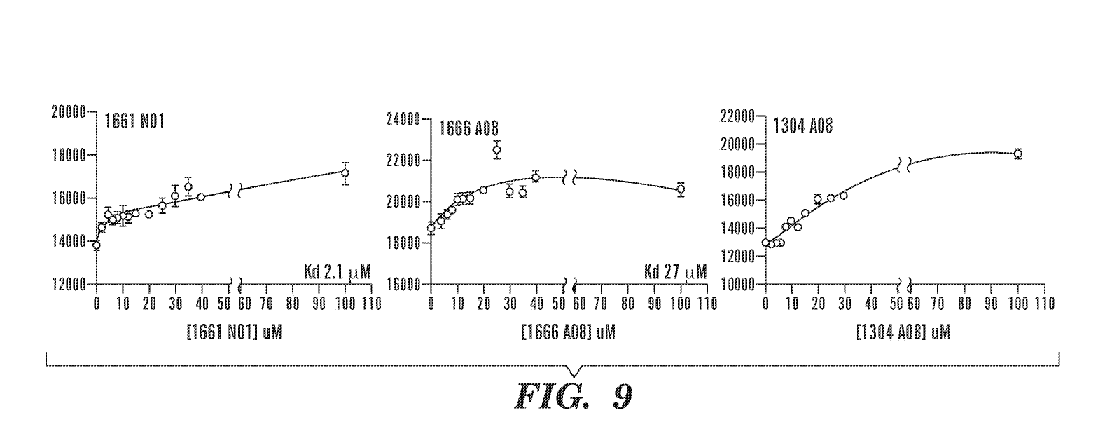

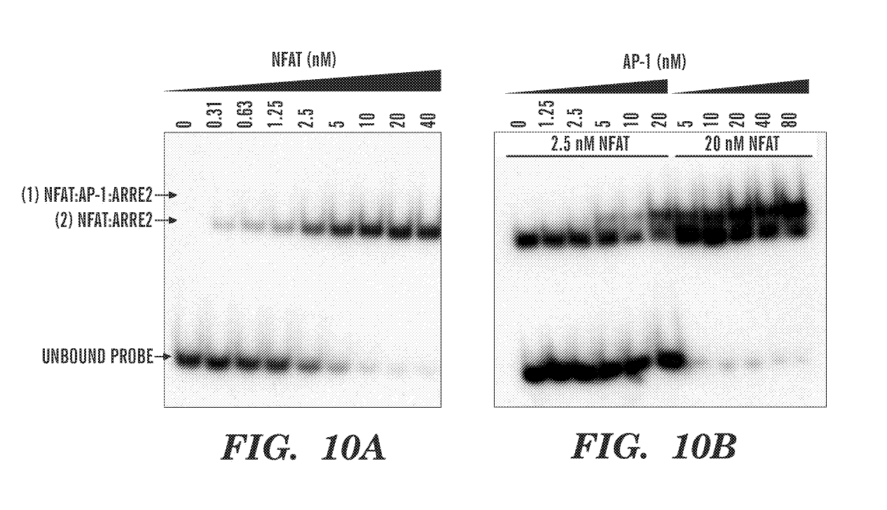

Inhibition of NFAT:AP-1:DNA complex formation in EMSAs: The inventors retested the hits in the primary assay—five compounds yielded consistent inhibition curves with half-maximal inhibitory concentrations in the range of ˜1.5-30 μM (FIGS. 3A, 3B and 9). The inventors further tested these compounds in EMSAs using a 32P end-labeled ARRE DNA probe with recombinant NFAT-DBD and Fos-Jun bZIP peptides (FIG. 3). A typical such EMSA yields a combination of three species, their relative amounts governed by the stoichiometry of individual protein and DNA components: the NFAT:AP-1:DNA complex (uppermost band, complex 1), NFAT:DNA complex (second band; complex 2) and free probe (lowest band) (FIG. 10). Compounds were added prior to addition of the labeled DNA probe, and hence before assembly of the ternary complex. Compounds 1668 P02 and 1661 N20 were found to modestly inhibit formation of the NFAT:AP-1:DNA complex, adjudged by the gradual disappearance of complex 1 and concomitant appearance of complex 2, along with free probe (FIG. 3). It can be noted that neither compound had an inhibitory effect on the NFAT:DNA complex even at high concentrations (6.25 mM; FIG. 3), confirming their inhibitory effect specifically on NFAT:AP-1:DNA assembly, albeit with modest efficacy. Of the remaining three primary hits, 1661 N01 and 1666 A08 also inhibited formation of the ternary complex while 1304 A08 appeared to inhibit both (data not shown).

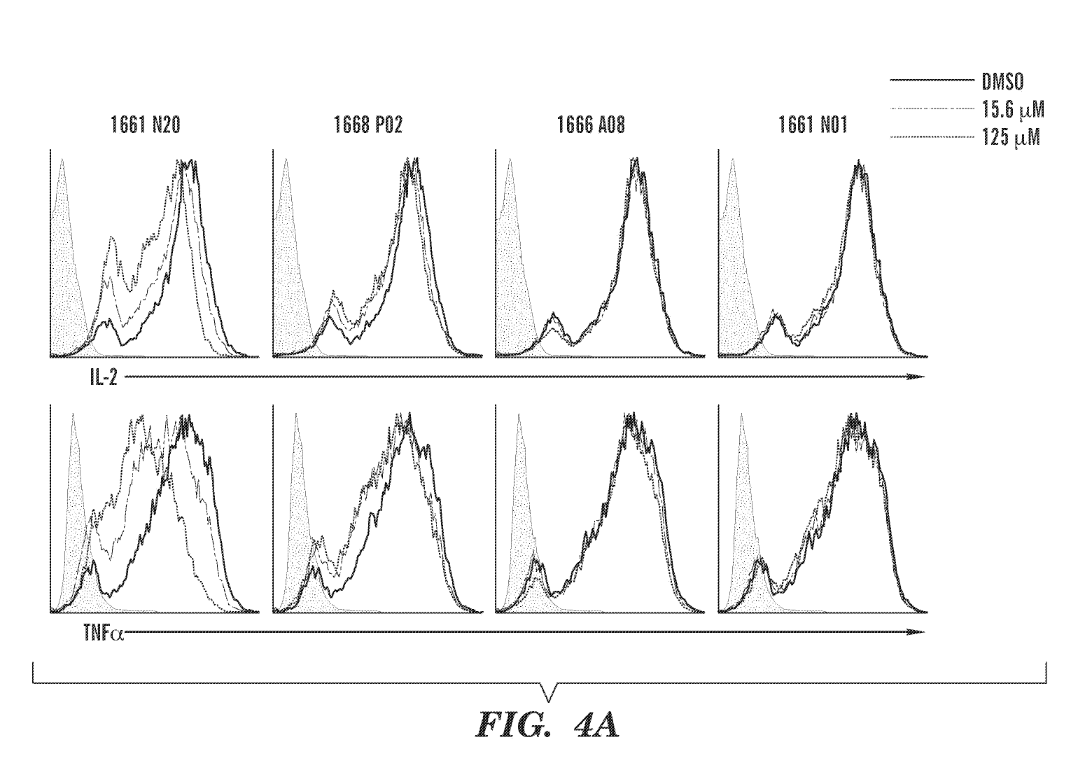

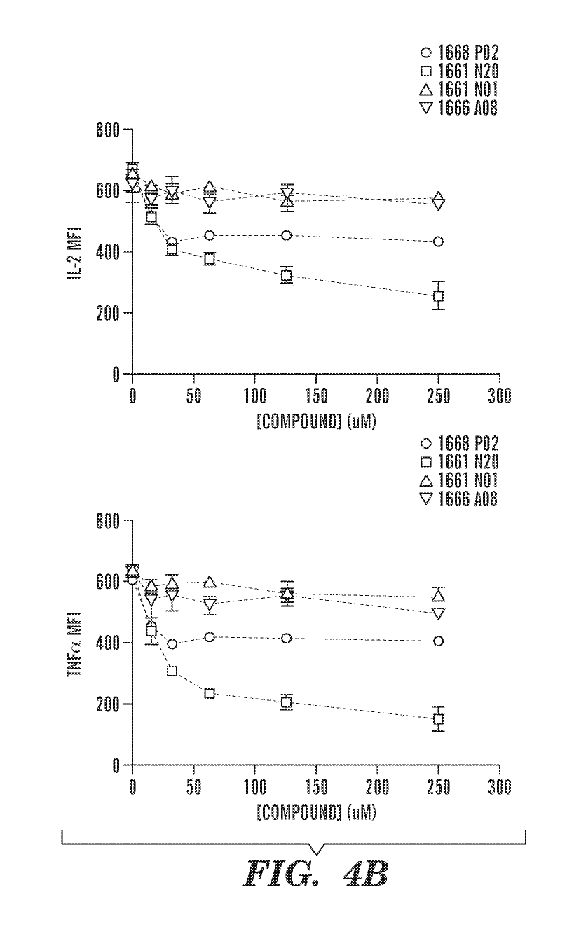

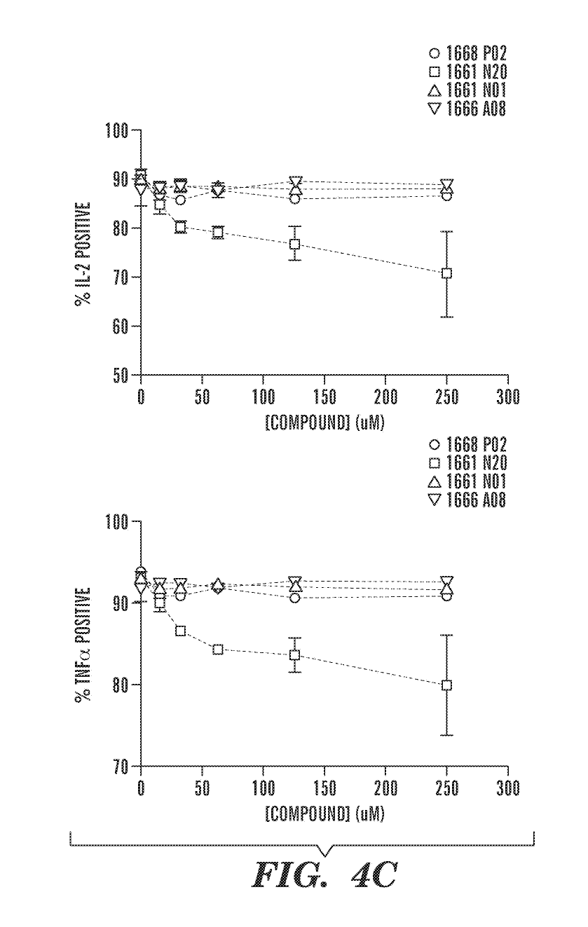

Inhibition of cytokine production: Next, the inventors added the found compounds to T cells being activated in the presence of 10 nM phorbol myristate acetate (PMA) and 500 nM ionomycin and measured the production of IL-2 and TNFα by intracellular cytokine FACS (FIG. 4). Only 1661 N20 and, to a lesser extent, 1668 P02, were found to be inhibitory (FIG. 4). Thus, further efforts were focused on these compounds.

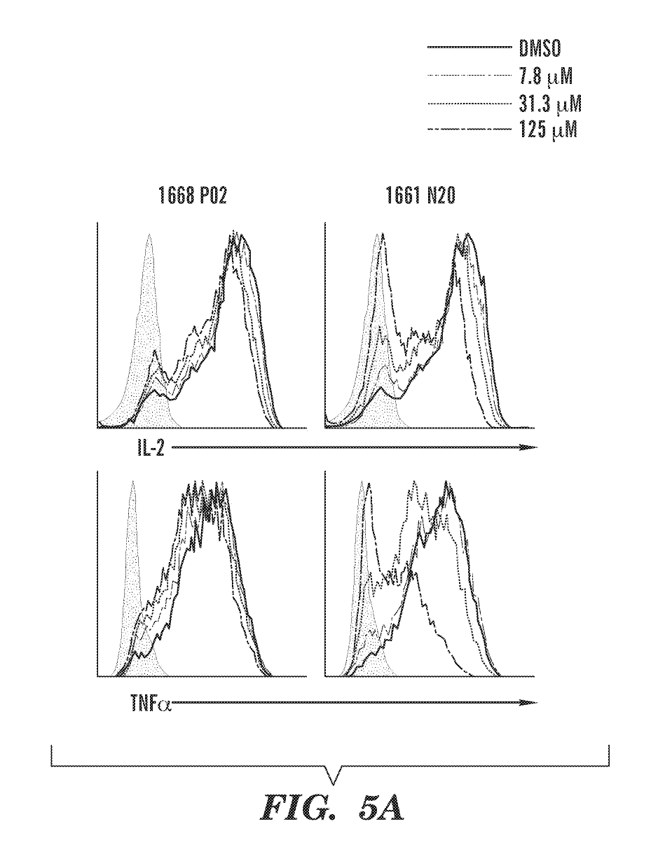

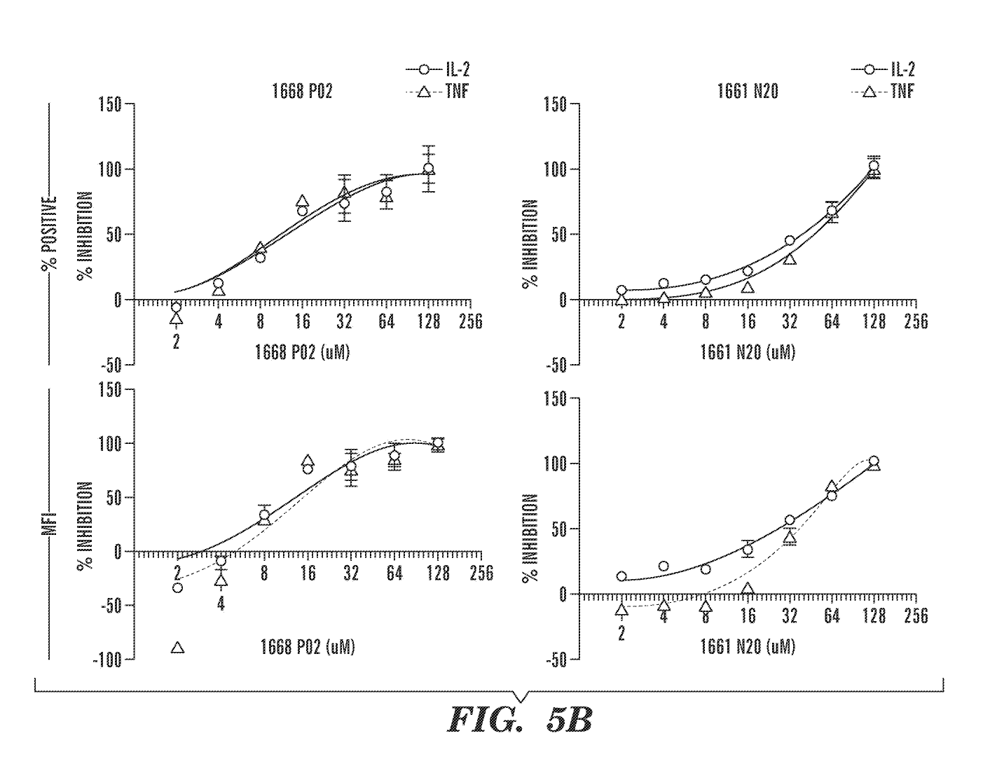

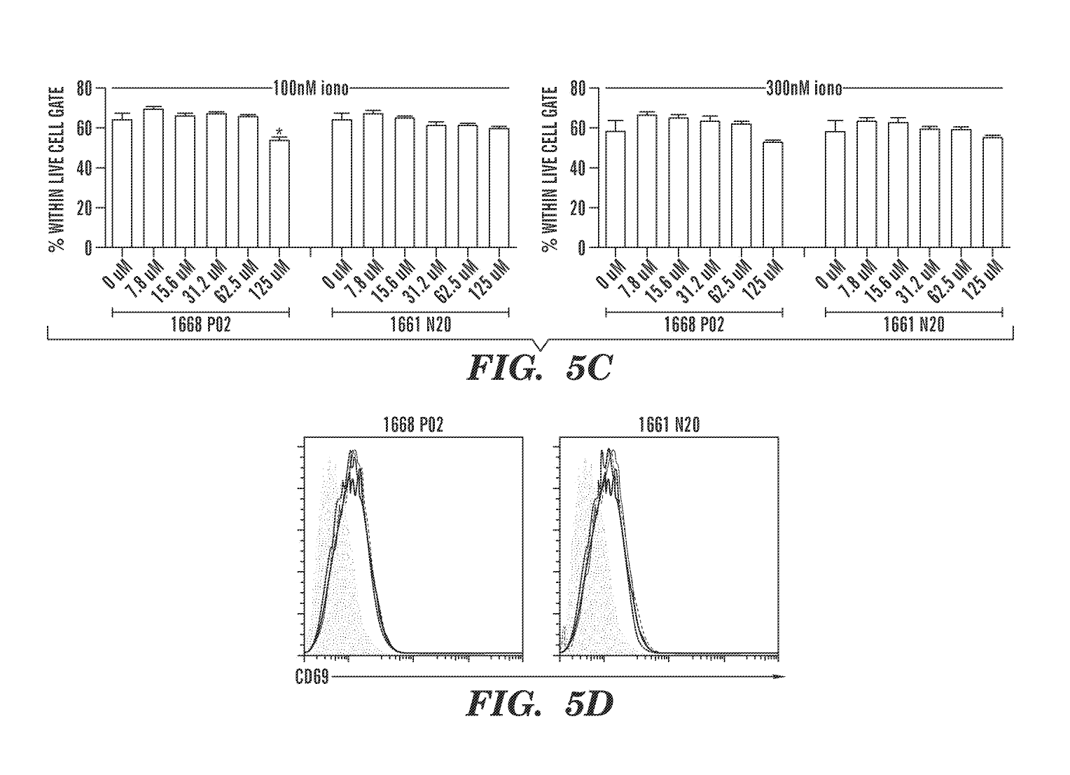

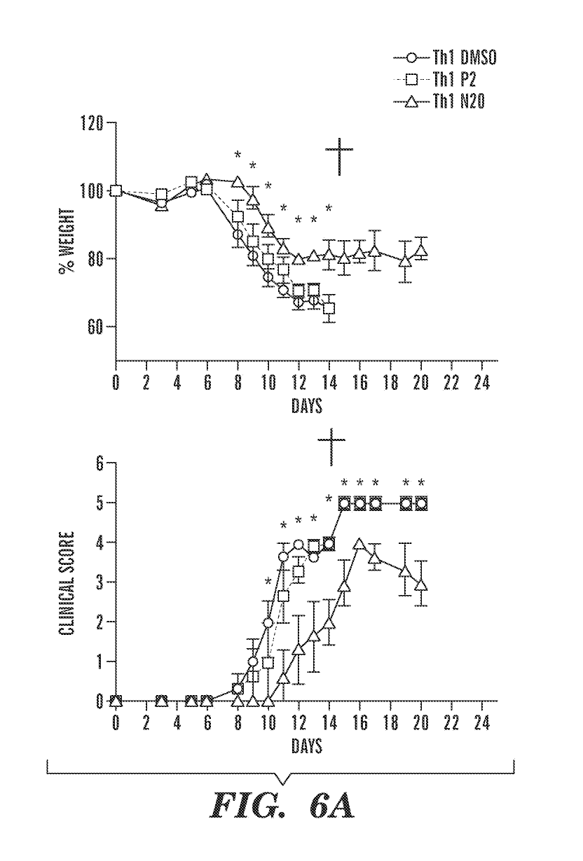

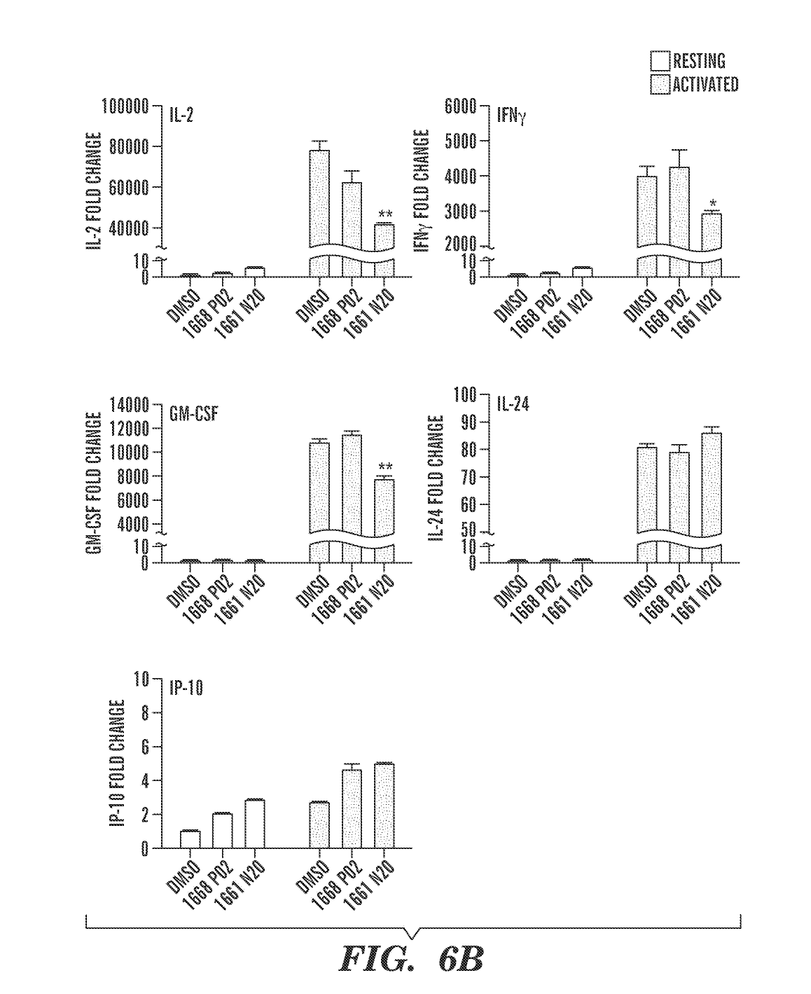

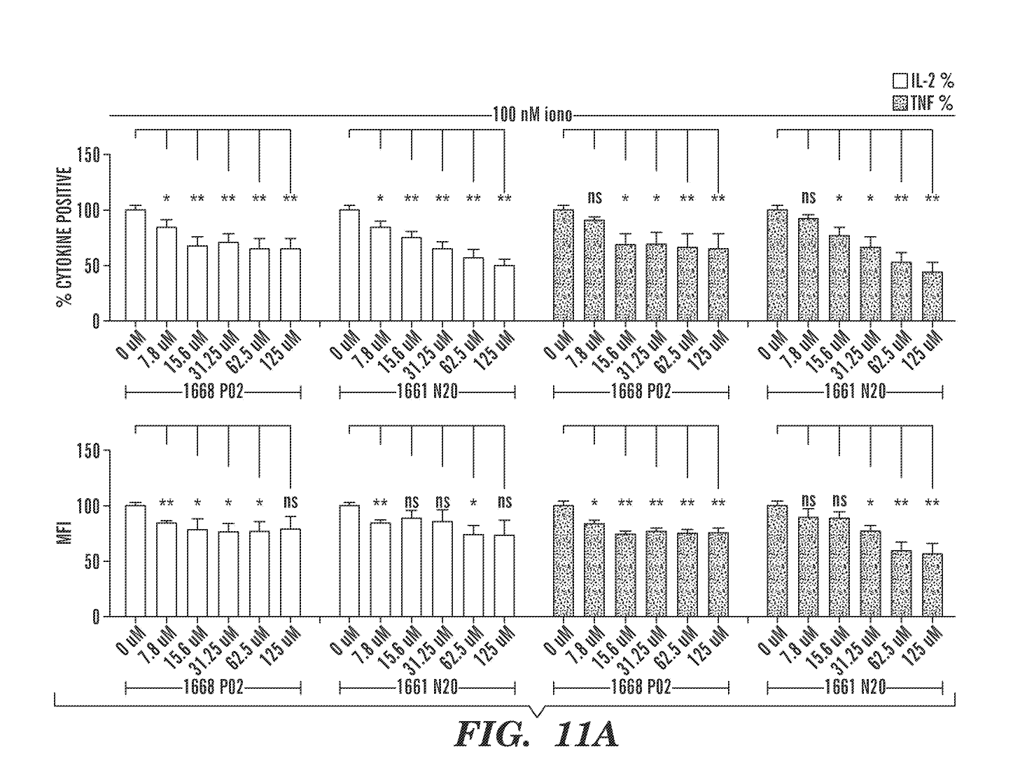

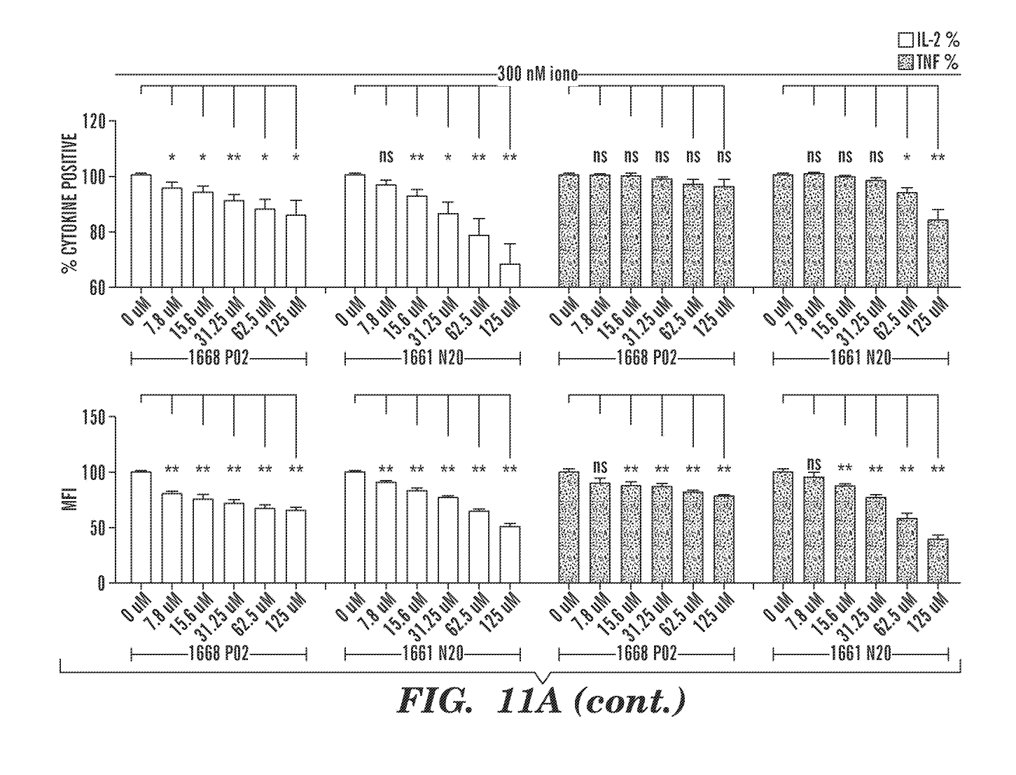

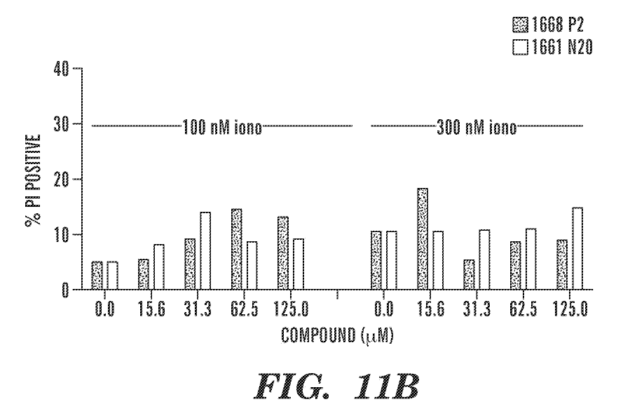

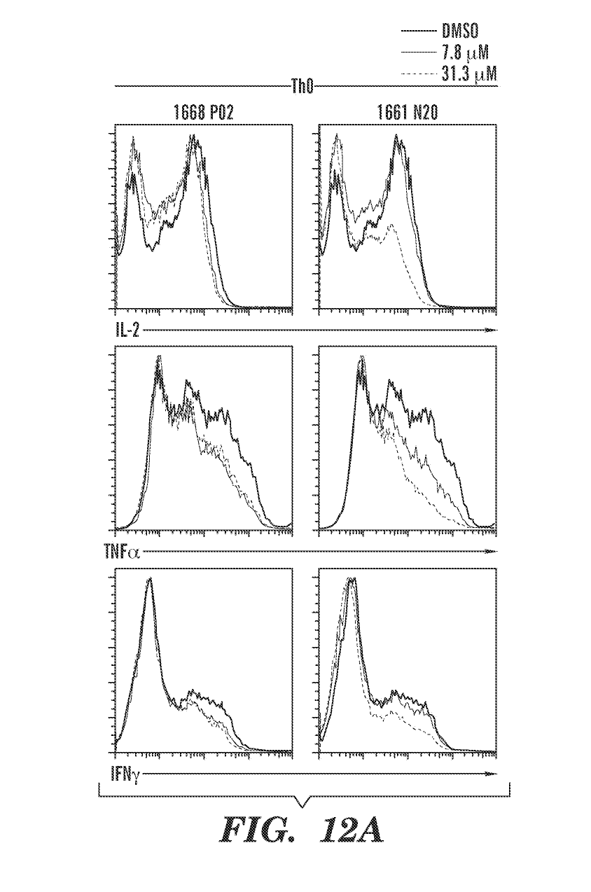

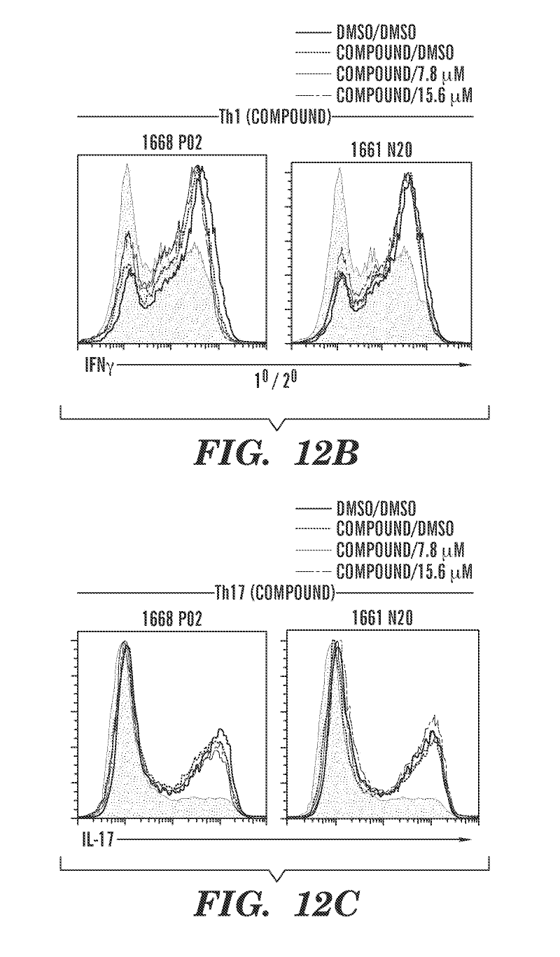

Surmising that they were at a plateau of activation, the inventors dropped the ionomycin concentration to either 100 nM or 300 nM and observed stronger effects on IL-2/TNFα production (up to a 50% reduction in MFIs of both IL-2 and TNFα with 1661 N20; FIGS. 5A 5B, and 11A); neither compound appeared to be cytotoxic at the concentrations tested and cell numbers within the live cell gate were unchanged. (FIGS. 5C and 11B). Upregulation of CD69, a Ca2+ independent gene, was unaffected by the presence of either compound during the 4 h activation period (FIG. 5D). Varying the T cell activation stimulus by using αCD3/αCD28 (instead of PMA/ionomycin) produced similar results, namely, acute inhibition of IL-2/TNFα production in the presence of 1661 N20 and 1668 P02 (FIG. 12A).

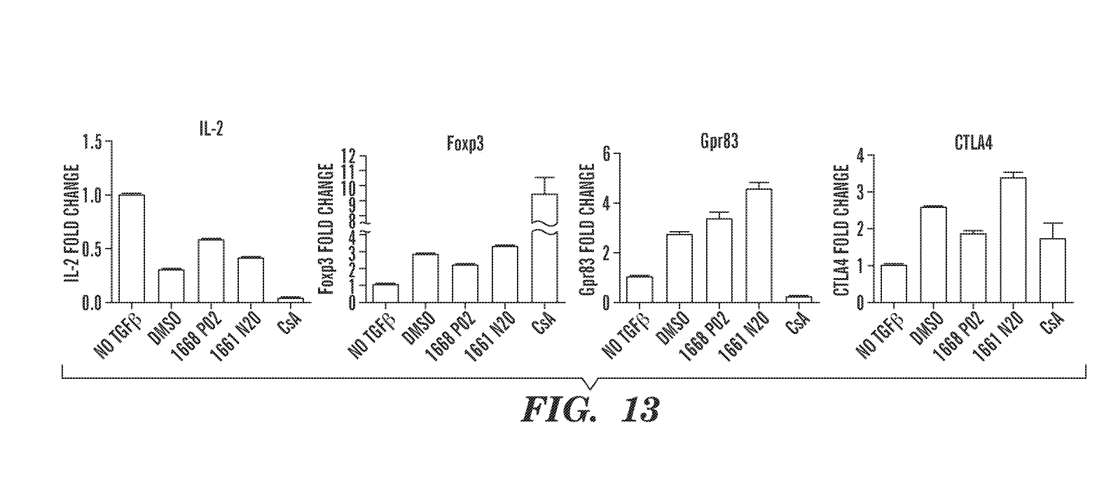

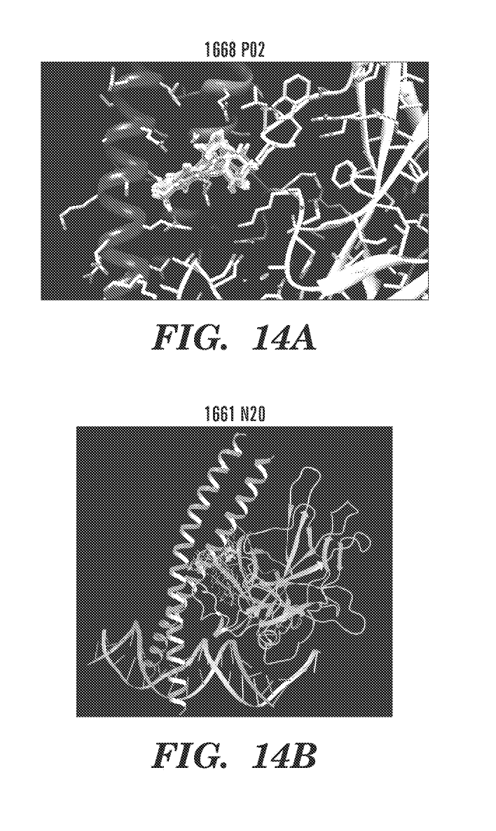

1661 N20 has an ameliorative effect on Th1-cell induced EAE: To study potential heritable effects of disrupting the NFAT:AP-1: DNA complex during primary T cell activation, the inventors treated 2D2 CD4 T cells (TCR transgenic for a peptide epitope from myelin oligodendrocyte glycoprotein (MOG)) with 7.8 μM of each compound during activation under Th1 conditions [7]. There was a slight decrease in the levels of IFNγ production in compound-treated T cells, compared to DMSO alone (FIG. 12B), while in CD4 T cells treated with compound during primary Th17 differentiation, IL-17 levels remained unchanged upon restimulation (FIG. 12C). Addition of 1668 P02 or 1661 N20 to T cells being activated in the presence of TGFβ, i.e., induced Treg (iTreg) polarizing conditions, did not affect the upregulation of Treg-associated genes, notably, Foxp3 (FIG. 13).

Next, the inventors used a passive transfer model of EAE [8] to ask if the relatively subtle in vitro effects of the compounds could translate into a potentially disease-modifying phenotype in vivo. CD4 T cells from 2D2 mice were isolated and differentiated under Th1 conditions in the presence of compound or DMSO, as described above. The cells were rested after primary activation and restimulated for 24 h with the corresponding compound or DMSO followed by intraperitoneal (IP) injections into irradiated C57BL/6 mice. This adoptive transfer model utilizing primed MOG-specific T cells represents the effector phase of EAE—the disease develops 5-6 days following IP injection, the earliest manifestation being a significant loss of weight, followed by rapid development of full-blown neuroinflammation as observed in classical MOG-induced EAE [1, 8] (FIG. 12A). Mice receiving the 1661 N20-treated CD4 Th1 cells developed disease with clearly delayed kinetics and also exhibited decreased severity of clinical symptoms—percentage weight loss and mean clinical score were significantly lower in this group compared to the 1668 P02- or DMSO-treated Th1 transfer groups (FIG. 12A). These latter two groups yielded overlapping disease curves (FIG. 12A).

Impaired production of proinflammatory mediators in 1661 N20-treated Th1 cells: Real time RT-PCRs with RNA from the T cells used for EAE induction showed a highly significant reduction in the amount of IL-2 mRNA transcript for the 1661 N20 group (p<0.001; Student's t-test), compared to DMSO controls; the decrease in IL-2 transcript levels in the 1668 P02 group was not statistically significant (p=0.08) (FIG. 12B). Transcript levels of two other proinflammatory cytokines, IL-24 and IP-10 [9, 10] did not decrease; IL-24 remained unchanged while IP-10 levels were actually higher in both the 1668 P02- and 1661 N20-treated cultures (FIG. 12B). GM-CSF, another gene known to be transcriptionally regulated by NFAT:AP-1 cooperation [5], has recently emerged as a major player in mediating inflammation during the effector phase of EAE [11]. GM-CSF transcript levels were significantly reduced in 1661 N20-treated Th1 cells relative to DMSO controls (p=0.003; Student's t-test), as were the levels of IFNγ mRNA (p=0.02; FIG. 12B). Without wishing to be bound by a theory, the observed ameliorative effect of 1661 N20 in this model of EAE, can be mediated, in part at least, by its inhibition of pro-inflammatory mediators and growth factors like IL-2, IFNγ and GM-CSF.

### REFERENCES

1. Bettelli, E., et al., Myelin oligodendrocyte glycoprotein-specific T cell receptor transgenic mice develop spontaneous autoimmune optic neuritis. J Exp Med, 2003. 197(9): p. 1073-81.
2. Bandukwala, H. S., et al., Structure of a domain-swapped FOXP3 dimer on DNA and its function in regulatory T cells. Immunity, 2011. 34(4): p. 479-91.
3. Chen, L., et al., Structure of the DNA-binding domains from NFAT, Fos and Jun bound specifically to DNA. Nature, 1998. 392(6671): p. 42-8.
4. Macian, F., C. Lopez-Rodriguez, and A. Rao, Partners in transcription: NFAT and AP-1. Oncogene, 2001. 20(19): p. 2476-89.
5. Hogan, P. G., et al., Transcriptional regulation by calcium, calcineurin, and NFAT. Genes Dev, 2003. 17(18): p. 2205-32.
6. Macian, F., C. Garcia-Rodriguez, and A. Rao, Gene expression elicited by NFAT in the presence or absence of cooperative recruitment of Fos and Jun. EMBO J, 2000. 19(17): p. 4783-95.
7. Djuretic, I. M., et al., Transcription factors T-bet and Runx3 cooperate to activate Ifng and silence Il4 in T helper type 1 cells. Nat Immunol, 2007. 8(2): p. 145-53.
8. Ghoreschi, K., et al., Generation of pathogenic T(H)17 cells in the absence of TGF-beta signalling. Nature, 2010. 467(7318): p. 967-71.
9. Ouyang, W., et al., Regulation and functions of the IL-10 family of cytokines in inflammation and disease. Annu Rev Immunol, 2011. 29: p. 71-109.
10. Lacotte, S., et al., CXCR3, inflammation, and autoimmune diseases. Ann N Y Acad Sci, 2009. 1173: p. 310-7.
11. Codarri, L., et al., RORgammat drives production of the cytokine GM-CSF in helper T cells, which is essential for the effector phase of autoimmune neuroinflammation. Nat Immunol, 2011. 12(6): p. 560-7.

All patents and other publications identified in the specification and examples are expressly incorporated herein by reference for all purposes. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Various embodiments of the invention are described above in the Detailed Description. While these descriptions directly describe the above embodiments, it is understood that those skilled in the art can conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it is obvious to those skilled in the art that, based upon the teachings herein, changes and modifications can be made without departing from this invention and its broader aspects and, therefore, the appended claims are to encompass within their scope all such changes and modifications as are within the true spirit and scope of this invention. It is understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

It is to be understood that this invention is not limited to the particular polymers, synthetic techniques, active agents, and the like as such can vary. It is also to be understood that the terminology used herein is for describing particular embodiments only, and is not intended to be limiting.

It must be noted that, as used in this specification, the singular forms “a,” “an,” and “the” include plural referents unless the context clearly dictates otherwise. Thus, for example, reference to an “excipient” includes a single excipient as well as two or more of the same or different excipients, reference to a “compound” includes a single compound as well as two or more compounds, and the like.

1. An assay for identifying a test compound that inhibits NFAT:AP-1:DNA interactions comprising the steps of:
forming a complex comprising a NFAT peptide, a Fos peptide, a Jun peptide, and an oligonucleotide, wherein the NFAT peptide comprises a DNA-binding domain (DBD), the Fos peptide and the Jun peptide each comprise a basic leucine zipper domain (bZIP domain), and the oligonucleotide comprises a NFAT binding site and a transcription factor binding site;
measuring the amount, formation or stability of the complex in the presence of a test compound relative to a reference sample; and
selecting the test compound that decreases the amount, formation or stability of the complex, wherein a decrease in amount, formation or stability of the complex in the presence of the test compound relative to the reference sample is indicative of a decrease in NFAT:AP-1:DNA interactions.

2-73. (canceled)

74. The assay of claim 1, wherein at least one component of the complex comprises a FRET donor and at least one other component of the complex comprises a FRET acceptor.

75. The assay of claim 74, wherein one of Fos peptide or the Jun peptide comprises the FRET donor.

76. The assay of claim 74, wherein oligonucleotide comprises the FRET acceptor.

77. The assay of claim 1, wherein the transcription site is an AP-1 binding site.

78. The assay of claim 1, wherein the sample further comprises a competitor of at least one component of the complex.

79. A method of inhibiting NFAT:AP-1:DNA interactions in a cell, the method comprising contacting a cell with a compound selected by the assay of any of claima 1 or 74-78.

80. The method of claim 79, wherein the compound is of Formula (I): 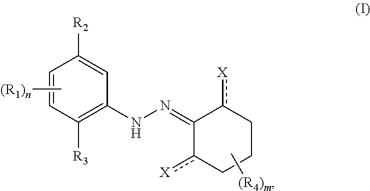
wherein
each R₁, R₂, R₃, and R₄ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; OR^{B}; —C(═O)R^{B}; —CO₂R^{B}; —C(═O)N(R^{B})₂; —CN; —SCN; —SR^{B}; —SOR^{B}; —SO₂R^{B}; —NO₂; —N(R^{B})₂; —NHC(O)R^{B}; or —C(R^{B})₃; wherein each occurrence of R^{B} is independently hydrogen; halogen; a protecting group; aliphatic; heteroaliphatic; acyl; aryl moiety; heteroaryl; hydroxyl; aloxy; aryloxy; alkylthioxy; arylthioxy; amino; alkylamino; dialkylamino; heteroaryloxy; heteroarylthioxy; alkylhalo;
each X is independently H, O, or R₄;
n is an integer 0-3, inclusive;
m is an integer 0-5, inclusive;
or salt thereof.

81. The method of claim 79, wherein the compound is of formula (II): 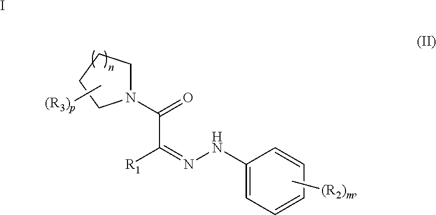
wherein
each R₁, R₂, and R₃, is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; OR^{B}; —C(═O)R^{B}; —CO₂R^{B}; —C(═O)N(R^{B})₂; —CN; —SCN; —SR^{B}; —SOR^{B}; —SO₂R^{B}; —NO₂; —N(R^{B})₂; —NHC(O)R^{B}; or —C(R^{B})₃; wherein each occurrence of R^{B} is independently hydrogen; halogen; a protecting group; aliphatic; heteroaliphatic; acyl; aryl moiety; heteroaryl; hydroxyl; aloxy; aryloxy; alkylthioxy; arylthioxy; amino; alkylamino; dialkylamino; heteroaryloxy; heteroarylthioxy; alkylhalo; and two R₂ can be taken together to form a five or six membered ring;
n is an integer 0-3, inclusive;
m is an integer 0-5, inclusive;
p is an integer 0-7, inclusive;
or salt thereof.

82. The method of claim 79, wherein the compound is

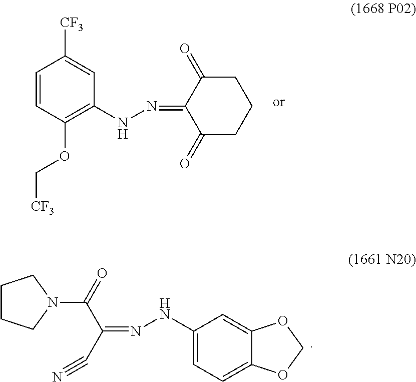 [SMILES: O=C1CCCC(=O)C1=NNc1cc(C(F)(F)F)ccc1OCC(F)(F)F ; N#C/C(=N/Nc1ccc2c(c1)OCO2)C(=O)N1CCCC1]

83. The method of claim 79, wherein said contact is in in vivo.

84. The method of claim 79, wherein said contact is in a subject, which subject suffers from a disease condition characterized by an elevated level immune response or the subject has elevated level of at least one pro-inflammatory mediator relative to a reference level.

85. A method of inhibiting an immune response in a subject, the method comprising administering a therapeutically effective amount of a compound selected by the method of claim 1 or 73-78 to a subject in need thereof.

86. The method of claim 85, wherein the compound is of formula (I): 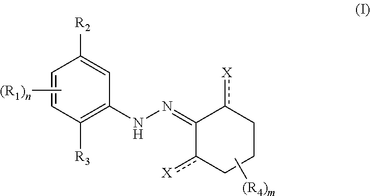
wherein
each R₁, R₂, R₃, and R₄ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; OR^{B}; —C(═O)R^{B}; —CO₂R^{B}; —C(═O)N(R^{B})₂; —CN; —SCN; —SR^{B}; —SOR^{B}; —SO₂R^{B}; —NO₂; —N(R^{B})₂; —NHC(O)R^{B}; or —C(R^{B})₃; wherein each occurrence of R^{B} is independently hydrogen; halogen; a protecting group; aliphatic; heteroaliphatic; acyl; aryl moiety; heteroaryl; hydroxyl; aloxy; aryloxy; alkylthioxy; arylthioxy; amino; alkylamino; dialkylamino; heteroaryloxy; heteroarylthioxy; alkylhalo;
each X is independently H, O, or R₄;
n is an integer 0-3, inclusive;
m is an integer 0-5, inclusive;
or salt thereof.

87. The method of claim 85, wherein the compound is of formula (II): 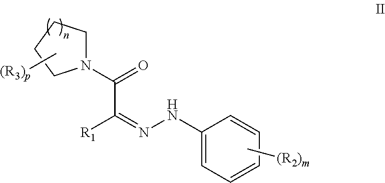
wherein
each R₁, R₂, and R₃, is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; OR^{B}; —C(═O)R^{B}; —CO₂R^{B}; —C(═O)N(R^{B})₂; —CN; —SCN; —SR^{B}; —SOR^{B}; —SO₂R^{B}; —NO₂; —N(R^{B})₂; —NHC(O)R^{B}; or —C(R^{B})₃; wherein each occurrence of R^{B} is independently hydrogen; halogen; a protecting group; aliphatic; heteroaliphatic; acyl; aryl moiety; heteroaryl; hydroxyl; aloxy; aryloxy; alkylthioxy; arylthioxy; amino; alkylamino; dialkylamino; heteroaryloxy; heteroarylthioxy; alkylhalo; and two R₂ can be taken together to form a five or six membered ring;
n is an integer 0-3, inclusive;
m is an integer 0-5, inclusive;
p is an integer 0-7, inclusive;
or salt thereof.

88. The method of claim 85, wherein the compound is

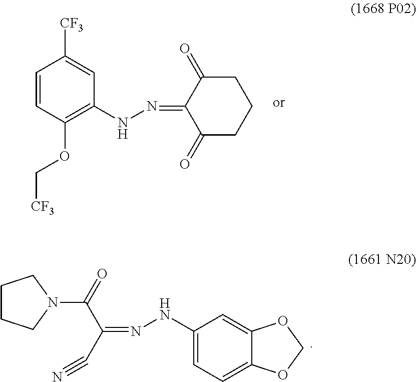 [SMILES: O=C1CCCC(=O)C1=NNc1cc(C(F)(F)F)ccc1OCC(F)(F)F ; N#C/C(=N/Nc1ccc2c(c1)OCO2)C(=O)N1CCCC1]

89. The method of claim 87, wherein the subject suffers from a disease condition characterized by an elevated level immune response or the subject has elevated level of at least one pro-inflammatory mediator relative to a reference level or the subject suffers from an inflammatory disease or disorder, cancer, or complications after a tissue/organ transplant.

90. The method of claim 89, wherein the inflammatory disease or disorder is an autoimmune disease.

91. The method of claim 87, the method further comprising co-administering a pharmaceutically active agent to the subject.

92. The method of claim 91, wherein the therapeutically active agent is an anti-inflammatory agent or an immune response modulator.
